(19) United States
(12) Patent Application Publication
(10) Pub. No.: US 2026/0337131 A1
(43) Pub. Date: Oct. 29, 2026

(54) METHODS OF MAKING AN ALKENE FROM A POLYMER

(71) Applicant: The Regents of the University of California, Oakland, CA (US)

(72) Inventors: John F. Hartwig, Berkeley, CA (US); Steven Hanna, Berkeley, CA (US); Richard J. Conk, Berkeley, CA (US)

(21) Appl. No.: 18/878,997

(22) PCT Filed: Jul. 14, 2023
(86) PCT No.: PCT/US2023/027840

§ 371 Date: Dec. 26, 2024

Related U.S. Application Data
(60) Provisional application No. 63/389,591, filed on Jul. 15, 2022.
(60) Provisional application No. 63/407,055, filed on Sep. 15, 2022.

(51) Int. Cl. C07C 5/27; C07C 4/06; C07C 5/333

(52) U.S. Cl. CPC C07C 5/2764; C07C 4/06; C07C 5/333

(57) ABSTRACT

Disclosed herein, inter alia, are methods of making alkenes from a polymer and dehydrogenated polyethylene compositions.

### CROSS-REFERENCES TO RELATED APPLICATIONS

This application claims priority to U.S. Provisional Application Nos. 63/389,591 filed Jul. 15, 2022 and 63/407,055 filed Sep. 15, 2022, the disclosures of which are incorporated herein by reference in their entireties and for all purposes.

### STATEMENT AS TO RIGHTS TO INVENTIONS MADE UNDER FEDERALLY SPONSORED RESEARCH AND DEVELOPMENT

This invention was made with government support under Grant Number DE-AC02-05CH11231 awarded by the Department of Energy. The government has certain rights in the invention.

### BACKGROUND

The conversion of polyolefins to small molecules (e.g., monomers) would enable the largest fraction of waste plastic to be a carbon feedstock, but such a task requires cleavage of carbon-carbon bonds that often resist selective chemical transformations. Disclosed herein, inter alia, are solutions to these and other problems in the art.

### BRIEF SUMMARY

In an aspect is provided a method of making an alkene, the method including mixing a dehydrogenated polymer with one or more olefin metathesis reagent(s). In embodiments, the method includes mixing a dehydrogenated polymer with one or more olefin metathesis reagent(s) and one or more isomerization reagent(s). In embodiments, the dehydrogenated polymer includes at most 10% unsaturated alkene monomer units. In embodiments, the dehydrogenated polymer includes at least 20 carbon atoms. In embodiments, prior to the mixing, the method includes mixing a polymer with one or more dehydrogenating reagent(s), thereby forming the dehydrogenated polymer. In embodiments, the method includes mixing a dehydrogenated polymer with one or more olefin metathesis reagent(s) and one or more isomerization reagent(s); wherein: (i) the dehydrogenated polymer includes at most 10% unsaturated alkene monomer units; (ii) the dehydrogenated polymer includes at least 20 carbon atoms; or (iii) prior to the mixing, the method includes mixing a polymer with one or more dehydrogenating reagent(s), thereby forming the dehydrogenated polymer.

In an aspect is provided a method of making a dehydrogenated polymer, the method including mixing a polymer with one or more dehydrogenating reagent(s), wherein: (i) a percentage of from 0.01% to 1.5% of monomer units in the dehydrogenated polymer are unsaturated. In embodiments, a percentage of from 1.7% to 2.3% of monomer units in the dehydrogenated polymer are unsaturated. In embodiments, a percentage of from 2.4% to 4.3% of monomer units in the dehydrogenated polymer are unsaturated. In embodiments, a percentage of from 4.5% to 13.2% of monomer units in the dehydrogenated polymer are unsaturated. In embodiments, the method includes mixing a polymer with one or more dehydrogenating reagent(s), wherein: (i) a percentage of from 0.01% to 1.5% or from 1.7% to 2.3% or from 2.4% to 4.3% or from 4.5% to 13.2% of monomer units in the dehydrogenated polymer are unsaturated; (ii) the dehydrogenated polymer has a number average molecular weight of about 2,200 Da or greater; (iii) the dehydrogenated polymer has a number average molecular weight of about 2,200 Da to about 6,500 Da and a percentage of 0.01% to 10% of monomer units are unsaturated; or (iv) the dehydrogenated polymer has a number average molecular weight of about 1,000 Da to about 500,000 Da and a percentage of from 0.01% to 1.5% or from 1.7% to 2.3% or from 2.4% to 4.3% or from 4.5% to 10% of monomer units in the dehydrogenated polymer are unsaturated. In embodiments, the dehydrogenated polymer has a number average molecular weight of about 100 Da to about 2,200 Da. In embodiments, the dehydrogenated polymer has a number average molecular weight of about 2,200 Da or greater. In embodiments, the dehydrogenated polymer has a number average molecular weight of about 2,200 Da to about 6,500 Da. In embodiments, the dehydrogenated polymer has a number average molecular weight of about 1,000 Da to about 500,000 Da.

In an aspect is provided a method of degrading a substance, the method including: (i) conducting one or more reaction(s) to form one or more olefin units in the substance; (ii) subjecting, after step (i), to conditions for olefin metathesis; and (iii) subjecting, after step (i), to conditions for olefin isomerization.

In an aspect is provided a method of making a substituted or unsubstituted alkene, the method including subjecting a dehydrogenated polymer to conditions for metathesis and to conditions for olefin isomerization.

In an aspect is provided a method of making a substituted or unsubstituted alkene, the method comprising subjecting a first substance to conditions for metathesis and to conditions for olefin isomerization; wherein: (i) the first substance includes at least 20 carbon atoms per molecule; or (ii) prior to subjecting the first substance to conditions for metathesis and to conditions for olefin isomerization, the method includes subjecting a second substance to conditions for dehydrogenation, thereby forming the first substance.

In an aspect is provided a substituted or unsubstituted dehydrogenated polyethylene, wherein a percentage of from 0.01% to 1.5% or from 1.7% to 2.3% or from 2.4% to 4.3% or from 4.5% to 13.2% of monomer units are unsaturated (e.g., olefinic, e.g., monoenes). In embodiments, a percentage of from 0.01% to 1.5% or from 1.7% to 2.3% or from 2.4% to 4.3% or from 4.5% to 13.2% of monomer units are olefinic. In embodiments, a percentage of from 0.01% to 1.5% of monomer units are olefinic. In embodiments, a percentage of from 1.7% to 2.3% of monomer units are olefinic. In embodiments, a percentage of from 1.7% to 2.3% of monomer units are olefinic. In embodiments, a percentage of from 2.4% to 4.3% of monomer units are olefinic. In embodiments, a percentage of from 4.5% to 13.2% of monomer units are olefinic.

In an aspect is provided a substituted or unsubstituted dehydrogenated polyethylene, having a number average molecular weight of 2,200 Da or greater.

### BRIEF DESCRIPTION OF THE DRAWINGS

FIGS. 1A-1D. Strategies for the valorization of PE waste. FIG. 1A: Degradation of PE by alkane metathesis with n-hexane (Huang 2016, Román-Leshkov 2021). FIG. 1B: Conversion of PE to alkyl aromatics by tandem hydrogenolysis/aromatization (Scott 2020). FIG. 1C: Degradation of PE by hydrogenolysis. FIG. 1D: Conversion of PE to propene by DIE (x=number of IE turnovers).

FIGS. 2A-2D. Conditions tested for DIE. FIG. 2A: Homogeneous dehydrogenation of PE. Conditions for dehydrogenation: 0.42 mol % Ir-^{tBu}POCOP, 0.46 mol % NaOtBu, 0.6M in PE, reactions run for 12 h with 0.4 equiv TBE (mol %, equiv TBE, and M of PE calculated relative to PE monomer units). FIG. 2B: Heterogeneous dehydrogenation of PE (HDPE, Mₙ=26.1 kDa) with bimetallic catalysts supported on alumina. Conditions: 20 wt % catalyst, 350° C. Catalysts were first activated under H₂ flow for 1 h, then dehydrogenation was run for 16 h under Ar flow. FIG. 2C: GPC Chromatogram of HDPE prior to and after ethenolysis with M-1. Conditions for ethenolysis were: 0.36 M in unsaturated PE 3.6 mol % M-1 in p-xylene, heated to 130° C. for 16 h under 25 bar ethylene. FIG. 2D: Sequential dehydrogenation and ethenolysis of a long chain paraffin (C₂₄H₅₀). Initial dehydrogenation yields olefins in 10% yield, and subsequent ethenolysis with M-1 yields a statistical distribution of shorter-chain olefins.

FIGS. 3A-3C. Development of conditions for DIE of PE. FIG. 3A: Optimization of catalyst combinations for IE of 1-octadecene. Conditions: 1-octadecene (0.5 M) was combined with isomerization (3.0 mol %) and metathesis (6.0 mol %) catalysts in p-xylene and heated to 60° C. under 25 bar of ethylene for 16 h. (a) I-2 (6 mol %) was combined with Pd(OAc)₂ (3 mol %) and methanesulfonic acid (30 mol %) to generate the active isomerization catalyst (25). The reaction was also conducted in THE to ensure catalyst solubility. (b) I-3 was activated by the addition of 1.5 equiv HBP in relative to I-3 (also conducted in THF for catalyst solubility). FIG. 3B: Application of IE to dehydro-polyethylene, with dehydro-HDPE yielding a maximum of 80% propene with 1.96% dehydrogenation. Conditions for IE of dehydrogenated PE: 3.6% M-1, 2.2% I-1, 1.78 M in PE, heated to 130° C. in p-xylene for 16 h under 25 bar of ethylene. FIG. 3C: DIE of ¹³C-labeled HDPE to check for background reactivity (conditions identical to those in FIG. 3B).

FIG. 4. DIE of post-consumer PE (conditions identical to those in FIG. 3B). Propene yields are reported as XX %(YY) where X is the yield obtained from polymer purified by precipitation from acetone, and Y is the yield obtained from unmodified polymer. Where a single yield is reported, dehydrogenation on unpurified samples failed. From left to right: Recycled HDPE (rHDPE) sourced from can tops; HDPE sourced from a milk jug; HDPE sourced from a shampoo bottle; LDPE sourced from food packaging.

FIGS. 5A-5E. Investigation of reaction scope and kinetics. FIG. 5A: Time course of IE with 1-octadecene (100% Yield=458.3 mBar). FIG. 5B: Time course of IE with dehydrogenated HDPE (1.41% Olefin) (100% Yield=193.5 mBar). FIG. 5C: Yields of IE on small molecules 1a-1d designed to assess the effect of branching on IE. FIG. 5D: Yields of IE on small molecules 1e-1f designed to test the effect of dienes on IE. FIG. 5E: Experiments utilizing poly(cyclooctene) PCO (Mₙ=designed to demonstrate the effect of polymer unsaturation on IE yield, in the absence of branching. Conditions for IE in FIGS. 5C-5E identical to those in FIG. 3C.

### DETAILED DESCRIPTION

### I. Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di-, and multivalent radicals. The alkyl may include a designated number of carbons (e.g., C₁-C₁₀ means one to ten carbons). Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkyl moiety may be fully saturated. An alkenyl may include more than one double bond and/or one or more triple bonds in addition to the one or more double bonds. An alkynyl may include more than one triple bond and/or one or more double bonds in addition to the one or more triple bonds.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene. The term “alkynylene” by itself or as part of another substituent, means, unless otherwise stated, a divalent radical derived from an alkyne.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., O, N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—S—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated. Examples of monocyclic cycloalkyls include cyclopropyl, cyclobutyl, cyclopentyl, cyclopentenyl, cyclohexyl, cyclohexenyl, cycloheptyl, and cyclooctyl. Bicyclic cycloalkyl ring systems are bridged monocyclic rings or fused bicyclic rings. In embodiments, bridged monocyclic rings contain a monocyclic cycloalkyl ring where two non adjacent carbon atoms of the monocyclic ring are linked by an alkylene bridge of between one and three additional carbon atoms (i.e., a bridging group of the form (CH₂)_{w}, where w is 1, 2, or 3). Representative examples of bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane. In embodiments, fused bicyclic cycloalkyl ring systems contain a monocyclic cycloalkyl ring fused to either a phenyl, a monocyclic cycloalkyl, a monocyclic cycloalkenyl, a monocyclic heterocyclyl, or a monocyclic heteroaryl. In embodiments, the bridged or fused bicyclic cycloalkyl is attached to the parent molecular moiety through any carbon atom contained within the monocyclic cycloalkyl ring. In embodiments, cycloalkyl groups are optionally substituted with one or two groups which are independently oxo or thia. In embodiments, the fused bicyclic cycloalkyl is a 5 or 6 membered monocyclic cycloalkyl ring fused to either a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the fused bicyclic cycloalkyl is optionally substituted by one or two groups which are independently oxo or thia. In embodiments, multicyclic cycloalkyl ring systems are a monocyclic cycloalkyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. In embodiments, the multicyclic cycloalkyl is attached to the parent molecular moiety through any carbon atom contained within the base ring. In embodiments, multicyclic cycloalkyl ring systems are a monocyclic cycloalkyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a monocyclic heteroaryl, a monocyclic cycloalkyl, a monocyclic cycloalkenyl, and a monocyclic heterocyclyl. Examples of multicyclic cycloalkyl groups include, but are not limited to tetradecahydrophenanthrenyl, perhydrophenothiazin-1-yl, and perhydrophenoxazin-1-yl.

In embodiments, a cycloalkyl is a cycloalkenyl. The term “cycloalkenyl” is used in accordance with its plain ordinary meaning. In embodiments, a cycloalkenyl is a monocyclic, bicyclic, or a multicyclic cycloalkenyl ring system. In embodiments, monocyclic cycloalkenyl ring systems are cyclic hydrocarbon groups containing from 3 to 8 carbon atoms, where such groups are unsaturated (i.e., containing at least one annular carbon carbon double bond), but not aromatic. Examples of monocyclic cycloalkenyl ring systems include cyclopentenyl and cyclohexenyl. In embodiments, bicyclic cycloalkenyl rings are bridged monocyclic rings or a fused bicyclic rings. In embodiments, bridged monocyclic rings contain a monocyclic cycloalkenyl ring where two non adjacent carbon atoms of the monocyclic ring are linked by an alkylene bridge of between one and three additional carbon atoms (i.e., a bridging group of the form (CH₂)_{w}, where w is 1, 2, or 3). Representative examples of bicyclic cycloalkenyls include, but are not limited to, norbornenyl and bicyclo[2.2.2]oct 2 enyl. In embodiments, fused bicyclic cycloalkenyl ring systems contain a monocyclic cycloalkenyl ring fused to either a phenyl, a monocyclic cycloalkyl, a monocyclic cycloalkenyl, a monocyclic heterocyclyl, or a monocyclic heteroaryl. In embodiments, the bridged or fused bicyclic cycloalkenyl is attached to the parent molecular moiety through any carbon atom contained within the monocyclic cycloalkenyl ring. In embodiments, cycloalkenyl groups are optionally substituted with one or two groups which are independently oxo or thia. In embodiments, multicyclic cycloalkenyl rings contain a monocyclic cycloalkenyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. In embodiments, the multicyclic cycloalkenyl is attached to the parent molecular moiety through any carbon atom contained within the base ring. In embodiments, multicyclic cycloalkenyl rings contain a monocyclic cycloalkenyl ring (base ring) fused to either (i) one ring system selected from the group consisting of a bicyclic cycloalkyl, a bicyclic cycloalkenyl, and a bicyclic heterocyclyl; or (ii) two ring systems independently selected from the group consisting of a monocyclic cycloalkyl, a monocyclic cycloalkenyl, and a monocyclic heterocyclyl.

In embodiments, a heterocycloalkyl is a heterocyclyl. The term “heterocyclyl” as used herein, means a monocyclic, bicyclic, or multicyclic heterocycle. The heterocyclyl monocyclic heterocycle is a 3, 4, 5, 6 or 7 membered ring containing at least one heteroatom independently selected from the group consisting of O, N, and S where the ring is saturated or unsaturated, but not aromatic. The 3 or 4 membered ring contains 1 heteroatom selected from the group consisting of O, N, and S. The 5 membered ring can contain zero or one double bond and one, two or three heteroatoms selected from the group consisting of O, N, and S. The 6 or 7 membered ring contains zero, one or two double bonds and one, two or three heteroatoms selected from the group consisting of O, N, and S. The heterocyclyl monocyclic heterocycle is connected to the parent molecular moiety through any carbon atom or any nitrogen atom contained within the heterocyclyl monocyclic heterocycle. Representative examples of heterocyclyl monocyclic heterocycles include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl. The heterocyclyl bicyclic heterocycle is a monocyclic heterocycle fused to either a phenyl, a monocyclic cycloalkyl, a monocyclic cycloalkenyl, a monocyclic heterocycle, or a monocyclic heteroaryl. The heterocyclyl bicyclic heterocycle is connected to the parent molecular moiety through any carbon atom or any nitrogen atom contained within the monocyclic heterocycle portion of the bicyclic ring system. Representative examples of bicyclic heterocyclyls include, but are not limited to, 2,3-dihydrobenzofuran-2-yl, 2,3-dihydrobenzofuran-3-yl, indolin-1-yl, indolin-2-yl, indolin-3-yl, 2,3-dihydrobenzothien-2-yl, decahydroquinolinyl, decahydroisoquinolinyl, octahydro-1H-indolyl, and octahydrobenzofuranyl. In embodiments, heterocyclyl groups are optionally substituted with one or two groups which are independently oxo or thia. In certain embodiments, the bicyclic heterocyclyl is a 5 or 6 membered monocyclic heterocyclyl ring fused to a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the bicyclic heterocyclyl is optionally substituted by one or two groups which are independently oxo or thia. Multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. The multicyclic heterocyclyl is attached to the parent molecular moiety through any carbon atom or nitrogen atom contained within the base ring. In embodiments, multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a monocyclic heteroaryl, a monocyclic cycloalkyl, a monocyclic cycloalkenyl, and a monocyclic heterocyclyl. Examples of multicyclic heterocyclyl groups include, but are not limited to 10H-phenothiazin-10-yl, 9,10-dihydroacridin-9-yl, 9,10-dihydroacridin-10-yl, 10H-phenoxazin-10-yl, 10,11-dihydro-5H-dibenzo[b,f]azepin-5-yl, 1,2,3,4-tetrahydropyrido[4,3-g]isoquinolin-2-yl, 12H-benzo[b]phenoxazin-12-yl, and dodecahydro-1H-carbazol-9-yl.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

A fused ring heterocyloalkyl-aryl is an aryl fused to a heterocycloalkyl. A fused ring heterocycloalkyl-heteroaryl is a heteroaryl fused to a heterocycloalkyl. A fused ring heterocycloalkyl-cycloalkyl is a heterocycloalkyl fused to a cycloalkyl. A fused ring heterocycloalkyl-heterocycloalkyl is a heterocycloalkyl fused to another heterocycloalkyl. Fused ring heterocycloalkyl-aryl, fused ring heterocycloalkyl-heteroaryl, fused ring heterocycloalkyl-cycloalkyl, or fused ring heterocycloalkyl-heterocycloalkyl may each independently be unsubstituted or substituted with one or more of the substitutents described herein.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g., substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g., all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The symbol “ [symbol] ” denotes the point of attachment of a chemical moiety to the remainder of a molecule or chemical formula.

The term “oxo,” as used herein, means an oxygen that is double bonded to a carbon atom.

The term “alkylarylene” as an arylene moiety covalently bonded to an alkylene moiety (also referred to herein as an alkylene linker). In embodiments, the alkylarylene group has the formula:

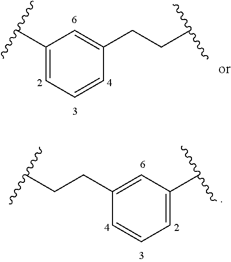 [SMILES: *CCc1cccc(*)c1 ; *CCc1cccc(*)c1]

An alkylarylene moiety may be substituted (e.g., with a substituent group) on the alkylene moiety or the arylene linker (e.g., at carbons 2, 3, 4, or 6) with halogen, oxo, —N₃, —CF₃, —CCl₃, —CBr₃, —CI₃, —CN, —C(O)H, —OH, —NH₂, —COOH, —CONH₂, —NO₂, —SH, —SO₂CH₃, —SO₃H, —OSO₃H, —SO₂NH₂, —NHNH₂, —ONH₂, —NHC(O)NHNH₂, substituted or unsubstituted C₁-C₅ alkyl or substituted or unsubstituted 2 to 5 membered heteroalkyl). In embodiments, the alkylarylene is unsubstituted.

The term “alkylsulfonyl,” as used herein, means a moiety having the formula —S(O₂)—R′, where R′ is a substituted or unsubstituted alkyl group as defined above. R′ may have a specified number of carbons (e.g., “C₁-C₄ alkylsulfonyl”).

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R″, —OC(O)R′, —C(O)R′, —CO₂R′, —CONR′R″, —OC(O)NR′R″, —NR″C(O)R′, —NR′—C(O)NR″R″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —NR′SO₂R″, —NR′C(O)R″, —NR′C(O)OR″, —NR′OR″, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)—OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R″″ groups when more than one of these groups is present.

Substituents for rings (e.g., cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g., a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur(S), phosphorus (P), and silicon (Si).

A “substituent group,” as used herein, means a group selected from the following moieties:
(A) oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(B) alkyl (e.g., C₁-C₂₀, C₁-C₁₂, C₁-C₈, C₁-C₆, C₁-C₄, or C₁-C₂), heteroalkyl (e.g., 2 to 20 membered, 2 to 12 membered, 2 to 8 membered, 2 to 6 membered, 4 to 6 membered, 2 to 3 membered, or 4 to 5 membered), cycloalkyl (e.g., C₃-C₁₀, C₃-C₈, C₃-C₆, C₄-C₆, or C₅-C₆), heterocycloalkyl (e.g., 3 to 10 membered, 3 to 8 membered, 3 to 6 membered, 4 to 6 membered, 4 to 5 membered, or 5 to 6 membered), aryl (e.g., C₆-C₁₂, C₆-C₁₀, or phenyl), or heteroaryl (e.g., 5 to 12 membered, 5 to 10 membered, 5 to 9 membered, or 5 to 6 membered), substituted with at least one substituent selected from:
   (i) oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁-C₈alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and (ii) alkyl (e.g., C₁-C₂₀, C₁-C₁₂, C₁-C₈, C₁-C₆, C₁-C₄, or C₁-C₂), heteroalkyl (e.g., 2 to 20 membered, 2 to 12 membered, 2 to 8 membered, 2 to 6 membered, 4 to 6 membered, 2 to 3 membered, or 4 to 5 membered), cycloalkyl (e.g., C₃-C₁₀, C₃-C₈, C₃-C₆, C₄-C₆, or C₅-C₆), heterocycloalkyl (e.g., 3 to 10 membered, 3 to 8 membered, 3 to 6 membered, 4 to 6 membered, 4 to 5 membered, or 5 to 6 membered), aryl (e.g., C₆-C₁₂, C₆-C₁₀, or phenyl), or heteroaryl (e.g., 5 to 12 membered, 5 to 10 membered, 5 to 9 membered, or 5 to 6 membered), substituted with at least one substituent selected from:
      (a) oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
      (b) alkyl (e.g., C₁-C₂₀, C₁-C₁₂, C₁-C₈, C₁-C₆, C₁-C₄, or C₁-C₂), heteroalkyl (e.g., 2 to 20 membered, 2 to 12 membered, 2 to 8 membered, 2 to 6 membered, 4 to 6 membered, 2 to 3 membered, or 4 to 5 membered), cycloalkyl (e.g., C₃-C₁₀, C₃-C₈, C₃-C₆, C₄-C₆, or C₅-C₆), heterocycloalkyl (e.g., 3 to 10 membered, 3 to 8 membered, 3 to 6 membered, 4 to 6 membered, 4 to 5 membered, or 5 to 6 membered), aryl (e.g., C₆-C₁₂, C₆-C₁₀, or phenyl), or heteroaryl (e.g., 5 to 12 membered, 5 to 10 membered, 5 to 9 membered, or 5 to 6 membered), substituted with at least one substituent selected from: oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted phenyl, and each substituted or unsubstituted heteroaryl is a substituted or unsubstituted 5 to 6 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound is a chemical species set forth in the application (e.g., Examples section, claims, embodiments, figures, or tables below).

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

In a recited claim or chemical formula description herein, each R substituent or L linker that is described as being “substituted” without reference as to the identity of any chemical moiety that composes the “substituted” group (also referred to herein as an “open substitution” on an R substituent or L linker or an “openly substituted” R substituent or L linker), the recited R substituent or L linker may, in embodiments, be substituted with one or more first substituent groups as defined below.

The first substituent group is denoted with a corresponding first decimal point numbering system such that, for example, R¹ may be substituted with one or more first substituent groups denoted by R^{1.1}, R² may be substituted with one or more first substituent groups denoted by R^{2.1}, R³ may be substituted with one or more first substituent groups denoted by R^{3.1}, R⁴ may be substituted with one or more first substituent groups denoted by R^{4.1}, R⁵ may be substituted with one or more first substituent groups denoted by R^{5.1}, and the like up to or exceeding an R¹⁰⁰ that may be substituted with one or more first substituent groups denoted by R^{100.1}. As a further example, R^{1A} may be substituted with one or more first substituent groups denoted by R^{1A.1}, R^{2A} may be substituted with one or more first substituent groups denoted by R^{2A.1}, R^{3A} may be substituted with one or more first substituent groups denoted by R^{3A.1}, R^{4A} may be substituted with one or more first substituent groups denoted by R^{4A.1}, R^{5A} may be substituted with one or more first substituent groups denoted by R^{5A.1} and the like up to or exceeding an R^{100A} may be substituted with one or more first substituent groups denoted by R^{100A.1}. As a further example, L¹ may be substituted with one or more first substituent groups denoted by R^{L1.1}, L² may be substituted with one or more first substituent groups denoted by R^{L2.1}, L³ may be substituted with one or more first substituent groups denoted by R^{13.1}, L⁴ may be substituted with one or more first substituent groups denoted by R^{14.1}, L⁵ may be substituted with one or more first substituent groups denoted by R^{L5.1} and the like up to or exceeding an L¹⁰⁰ which may be substituted with one or more first substituent groups denoted by R^{L100.1}. Thus, each numbered R group or L group (alternatively referred to herein as R^{WW} or L^{WW} wherein “WW” represents the stated superscript number of the subject R group or L group) described herein may be substituted with one or more first substituent groups referred to herein generally as R^{WW.1} or R^{LWW.1}, respectively. In turn, each first substituent group (e.g., R^{1.1}, R^{2.1}, R^{3.1}, R^{4.1}, R^{5.1} . . . . R^{100.1}; R^{1A.1}, R^{2A.1}, R^{3A.1}, R^{4A.1}, R^{5A.1} . . . R^{100A.1}; R^{L1.1}, R^{L2.1}, R^{L3.1}, R^{L4.1}, R^{L5.1}. R^{L100.1}) may be further substituted with one or more second substituent groups (e.g., R^{1.2}, R^{2.2}, R^{3.2}, R^{4.2}, R^{5.2} . . . . R^{100.2}; R^{1A.2}, R^{2A.2}, R^{3A.2}, R^{4A.2}, R^{5A.2} . . . . R^{100A.2}; R^{L1.2}, R^{L2.2}, R^{L3.2}, R^{L4.2}, R^{L5.2}. R^{L100.2}, respectively). Thus, each first substituent group, which may alternatively be represented herein as R^{WW.1} as described above, may be further substituted with one or more second substituent groups, which may alternatively be represented herein as R^{WW.2}.

Finally, each second substituent group (e.g., R^{1.2}, R^{2.2}, R^{3.2}, R^{4.2}, R^{5.2} . . . R^{100.2}, R^{1A.2}, R^{2A.2}, R^{3A.2}, R^{4A.2}, R^{5A.2} . . . . R^{100A.2}; R^{L1.2}, R^{12.2}, R^{L3.2}, R^{L4.2}, R^{L5.2} . . . . R^{L100.2}) may be further substituted with one or more third substituent groups (e.g., R^{1.3}, R^{2.3}, R^{3.3}, R^{4.3}, R^{5.3} . . . . R^{100.3}. R^{1A.3}, R^{2A.3}, R^{3A.3}, R^{4A.3}, R^{5A.3} . . . R^{100A.3}. R^{L1.3}, R^{L2.3}, R^{L3.3}, R^{L4.3}, R^{L5.3} . . . . R^{L100.3}, respectively). Thus, each second substituent group, which may alternatively be represented herein as R^{WW.2} as described above, may be further substituted with one or more third substituent groups, which may alternatively be represented herein as R^{WW.3}. Each of the first substituent groups may be optionally different. Each of the second substituent groups may be optionally different. Each of the third substituent groups may be optionally different.

Thus, as used herein, R^{WW} represents a substituent recited in a claim or chemical formula description herein which is openly substituted. “WW” represents the stated superscript number of the subject R group (1, 2, 3, 1A, 2A, 3A, 1B, 2B, 3B, etc.). Likewise, L^{WW} is a linker recited in a claim or chemical formula description herein which is openly substituted. Again, “WW” represents the stated superscript number of the subject L group (1, 2, 3, 1A, 2A, 3A, 1B, 2B, 3B, etc.). As stated above, in embodiments, each R^{WW} may be unsubstituted or independently substituted with one or more first substituent groups, referred to herein as R^{WW.1}; each first substituent group, R^{WW.1}, may be unsubstituted or independently substituted with one or more second substituent groups, referred to herein as R^{WW.2}, and each second substituent group may be unsubstituted or independently substituted with one or more third substituent groups, referred to herein as R^{WW.3}. Similarly, each L^{WW} linker may be unsubstituted or independently substituted with one or more first substituent groups, referred to herein as R^{LWW.1}; each first substituent group, R^{LWW.1}, may be unsubstituted or independently substituted with one or more second substituent groups, referred to herein as R^{LWW.2}; and each second substituent group may be unsubstituted or independently substituted with one or more third substituent groups, referred to herein as R^{LWW.3}. Each first substituent group is optionally different. Each second substituent group is optionally different. Each third substituent group is optionally different. For example, if R^{WW} is phenyl, the said phenyl group is optionally substituted by one or more R^{WW.1} groups as defined herein below, e.g., when R^{WW.1} is R^{WW.2}-substituted or unsubstituted alkyl, examples of groups so formed include but are not limited to itself optionally substituted by 1 or more R^{WW.2}, which R^{WW.2} is optionally substituted by one or more R^{WW.3}. By way of example when the R^{WW} group is phenyl substituted by R^{WW.1}, which is methyl, the methyl group may be further substituted to form groups including but not limited to:

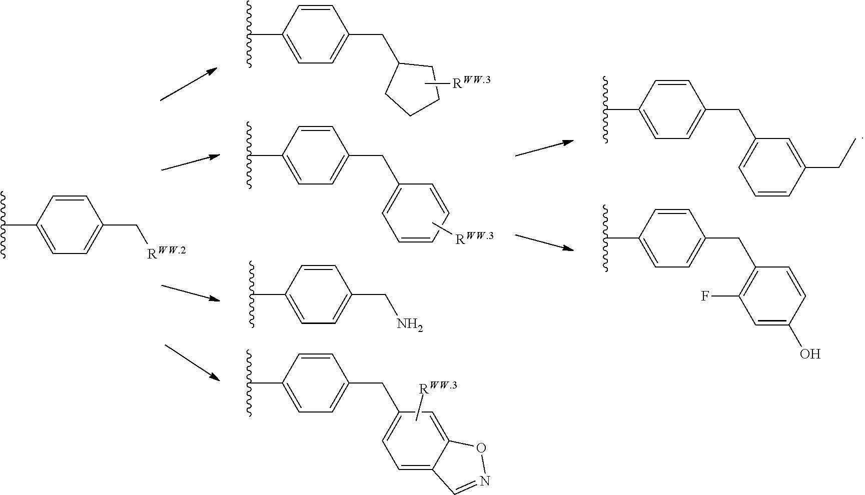

R^{WW.1} is independently oxo, halogen, —CX^{WW.1}₃, —CHX^{WW.1}₂, —CH₂X^{WW.1}, —OCX^{WW.1}₃, —OCH₂X^{WW.1}, —OCHX^{WW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, R^{WW.2}-substituted or unsubstituted alkyl (e.g., C₁-C₈, C₁-C₆, C₁-C₄, or C₁-C₂), R^{WW.2}-substituted or unsubstituted heteroalkyl (e.g., 2 to 8 membered, 2 to 6 membered, 4 to 6 membered, 2 to 3 membered, or 4 to 5 membered), R^{WW.2}-substituted or unsubstituted cycloalkyl (e.g., C₃-C₈, C₃-C₆, C₄-C₆, or C₅-C₆), R^{WW.2}-substituted or unsubstituted heterocycloalkyl (e.g., 3 to 8 membered, 3 to 6 membered, 4 to 6 membered, 4 to 5 membered, or 5 to 6 membered), R^{WW.2}-substituted or unsubstituted aryl (e.g., C₆-C₁₂, C₆-C₁₀, or phenyl), or R^{WW.2}-substituted or unsubstituted heteroaryl (e.g., 5 to 12 membered, 5 to 10 membered, 5 to 9 membered, or 5 to 6 membered). In embodiments, R^{WW.1} is independently oxo, halogen, —CX^{WW.1}₃, —CHX^{WW.1}₂, —CH₂X^{WW.1}, —OCX^{WW.1}₃, —OCH₂X^{WW.1}, —OCHX^{WW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.1} is independently —F, —Cl, —Br, or —I.

R^{WW.2} is independently oxo, halogen, —CX^{WW.2}₃, —CHX^{WW.2}₂, —CH₂X^{WW.2}, —OCX^{WW.2}₃, —OCH₂X^{WW.2}, —OCHX^{WW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, R^{WW.3}-substituted or unsubstituted alkyl (e.g., C₁-C₈, C₁-C₆, C₁-C₄, or C₁-C₂), R^{WW.3}-substituted or unsubstituted heteroalkyl (e.g., 2 to 8 membered, 2 to 6 membered, 4 to 6 membered, 2 to 3 membered, or 4 to 5 membered), R^{WW.3}-substituted or unsubstituted cycloalkyl (e.g., C₃-C₈, C₃-C₆, C₄-C₆, or C₅-C₆), R^{WW.3}-substituted or unsubstituted heterocycloalkyl (e.g., 3 to 8 membered, 3 to 6 membered, 4 to 6 membered, 4 to 5 membered, or 5 to 6 membered), R^{WW.3}-substituted or unsubstituted aryl (e.g., C₆-C₁₂, C₆-C₁₀, or phenyl), or R^{WW.3}-substituted or unsubstituted heteroaryl (e.g., 5 to 12 membered, 5 to 10 membered, 5 to 9 membered, or 5 to 6 membered). In embodiments, R^{WW.2} is independently oxo, halogen, —CX^{WW.2}₃, —CHX^{WW.2}₂, —CH₂X^{WW.2}, —OCX^{WW.2}₃, —OCH₂X^{WW.2}, —OCHX^{WW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.2} is independently —F, —Cl, —Br, or —I.

R^{WW.3} is independently oxo, halogen, —CX^{WW.3}₃, —CHX^{WW.3}₂, —CH₂X^{WW.3}, —OCX^{WW.3}₃, —OCH₂X^{WW.3}, —OCHX^{WW.3}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.3} is independently —F, —Cl, —Br, or —I.

Where two different R^{WW} substituents are joined together to form an openly substituted ring (e.g., substituted cycloalkyl, substituted heterocycloalkyl, substituted aryl or substituted heteroaryl), in embodiments the openly substituted ring may be independently substituted with one or more first substituent groups, referred to herein as R^{WW.1}; each first substituent group, R^{WW.1}, may be unsubstituted or independently substituted with one or more second substituent groups, referred to herein as R^{WW.2}; and each second substituent group, R^{WW.2}, may be unsubstituted or independently substituted with one or more third substituent groups, referred to herein as R^{WW.3}, and each third substituent group, R^{WW.3}, is unsubstituted. Each first substituent group is optionally different. Each second substituent group is optionally different. Each third substituent group is optionally different. In the context of two different R^{WW} substituents joined together to form an openly substituted ring, the “WW” symbol in the R^{WW.1}, R^{WW.2} and R^{WW.3} refers to the designated number of one of the two different R^{WW} substituents. For example, in embodiments where R^{100A} and R^{100B} are optionally joined together to form an openly substituted ring, R^{WW.1} is R^{100A.1}, R^{WW.2} is R^{100A.2}, and R^{WW.3} is R^{100A.3}. Alternatively, in embodiments where R^{100A} and R^{100B} are optionally joined together to form an openly substituted ring, R^{WW.1} is R^{100B.1}, R^{WW.2} is R^{100B.2}, and R^{WW.3} is R^{100B.3}. R^{WW.1}, R^{WW.2} and R^{WW.3} in this paragraph are as defined in the preceding paragraphs.

R^{LWW.1} is independently oxo, halogen, —CX^{LWW.1}₃, —CHX^{LWW.1}₂, —CH₂X^{LWW.1}, —OCX^{LWW.1}₃, —OCH₂X^{LWW.1}, —OCHX^{LWW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, R^{LWW.2}-substituted or unsubstituted alkyl (e.g., C₁-C₈, C₁-C₆, C₁-C₄, or C₁-C₂), R^{LWW.2}-substituted or unsubstituted heteroalkyl (e.g., 2 to 8 membered, 2 to 6 membered, 4 to 6 membered, 2 to 3 membered, or 4 to 5 membered), R^{LWW.2}-substituted or unsubstituted cycloalkyl (e.g., C₃-C₈, C₃-C₆, C₄-C₆, or C₅-C₆), R^{LWW.2}-substituted or unsubstituted heterocycloalkyl (e.g., 3 to 8 membered, 3 to 6 membered, 4 to 6 membered, 4 to 5 membered, or 5 to 6 membered), R^{LWW.2}-substituted or unsubstituted aryl (e.g., C₆-C₁₂, C₆-C₁₀, or phenyl), or R^{LWW.2}-substituted or unsubstituted heteroaryl (e.g., 5 to 12 membered, 5 to 10 membered, 5 to 9 membered, or 5 to 6 membered). In embodiments, R^{LWW.1} is independently oxo, halogen, —CX^{LWW.1}₃, —CHX^{LWW.1}₂, —CH₂X^{LWW.1}, —OCX^{LWW.1}₃, —OCH₂X^{LWW.1}, —OCHX^{LWW.1}, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.1} is independently —F, —Cl, —Br, or —I.

R^{LWW.2} is independently oxo, halogen, —CX^{LWW.2}₃, —CHX^{LWW.2}₂, —CH₂X^{LWW.2}, —OCX^{LWW.2}₃, —OCH₂X^{LWW.2}, —OCHX^{LWW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, R^{LWW.3}-substituted or unsubstituted alkyl (e.g., C₁-C₈, C₁-C₆, C₁-C₄, or C₁-C₂), R^{LWW.3}-substituted or unsubstituted heteroalkyl (e.g., 2 to 8 membered, 2 to 6 membered, 4 to 6 membered, 2 to 3 membered, or 4 to 5 membered), R^{WW.3}-substituted or unsubstituted cycloalkyl (e.g., C₃-C₈, C₃-C₆, C₄-C₆, or C₅-C₆), R^{LWW.3}-substituted or unsubstituted heterocycloalkyl (e.g., 3 to 8 membered, 3 to 6 membered, 4 to 6 membered, 4 to 5 membered, or 5 to 6 membered), R^{LWW.3}-substituted or unsubstituted aryl (e.g., C₆-C₁₂, C₆-C₁₀, or phenyl), or R^{LWW.3}-substituted or unsubstituted heteroaryl (e.g., 5 to 12 membered, 5 to 10 membered, 5 to 9 membered, or 5 to 6 membered). In embodiments, R^{LWW.2} is independently oxo, halogen, —CX^{LWW.2}₃, —CHX^{LWW.2}₂, —CH₂X^{LWW.2}, —OCX^{LWW.2}₃, —OCH₂X^{LWW.2}, —OCHX^{LWW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.2} is independently —F, —Cl, —Br, or —I.

R^{LWW.3} is independently oxo, halogen, —CX^{LWW.3}₃, —CHX^{LWW.3}₂, —CH₂X^{LWW.3}, —OCX^{LWW.3}₃, —OCH₂X^{LWW.3}, —OCHX^{LWW.3}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.3} is independently —F, —Cl, —Br, or —I.

In the event that any R group recited in a claim or chemical formula description set forth herein (R^{WW} substituent) is not specifically defined in this disclosure, then that R group (R^{WW} group) is hereby defined as independently oxo, halogen, —CX^{WW}₃, —CHX^{WW}₂, —CH₂X^{WW}, —OCX^{WW}₃, —OCH₂X^{WW}, —OCHX^{WW}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, R^{WW.1}-substituted or unsubstituted alkyl (e.g., C₁-C₈, C₁-C₆, C₁-C₄, or C₁-C₂), R^{WW.1}-substituted or unsubstituted heteroalkyl (e.g., 2 to 8 membered, 2 to 6 membered, 4 to 6 membered, 2 to 3 membered, or 4 to 5 membered), R^{WW.1}-substituted or unsubstituted cycloalkyl (e.g., C₃-C₈, C₃-C₆, C₄-C₆, or C₅-C₆), R^{WW.1}-substituted or unsubstituted heterocycloalkyl (e.g., 3 to 8 membered, 3 to 6 membered, 4 to 6 membered, 4 to 5 membered, or 5 to 6 membered), R^{WW.1}-substituted or unsubstituted aryl (e.g., C₆-C₁₂, C₆-C₁₀, or phenyl), or R^{WW.1}-substituted or unsubstituted heteroaryl (e.g., 5 to 12 membered, 5 to 10 membered, 5 to 9 membered, or 5 to 6 membered). X^{WW} is independently —F, —Cl, —Br, or —I. Again, “WW” represents the stated superscript number of the subject R group (e.g., 1, 2, 3, 1A, 2A, 3A, 1B, 2B, 3B, etc.). R^{WW.1}, R^{WW.2}, and R^{WW.3} are as defined above.

In the event that any L linker group recited in a claim or chemical formula description set forth herein (i.e., an L^{WW} substituent) is not explicitly defined, then that L group (L^{WW} group) is herein defined as independently a bond, —O—, —NH—, —C(O)—, —C(O)NH—, —NHC(O)—, —NHC(O)NH—, —C(O)O—, —OC(O)—, —S—, —SO₂—, —SO₂NH—, R^{LWW.1}-substituted or unsubstituted alkylene (e.g., C₁-C₈, C₁-C₆, C₁-C₄, or C₁-C₂), R^{LWW.1}-substituted or unsubstituted heteroalkylene (e.g., 2 to 8 membered, 2 to 6 membered, 4 to 6 membered, 2 to 3 membered, or 4 to 5 membered), R^{LWW.1}-substituted or unsubstituted cycloalkylene (e.g., C₃-C₈, C₃-C₆, C₄-C₆, or C₅-C₆), R^{LWW.1}-substituted or unsubstituted heterocycloalkylene (e.g., 3 to 8 membered, 3 to 6 membered, 4 to 6 membered, 4 to 5 membered, or 5 to 6 membered), R^{LWW.1}-substituted or unsubstituted arylene (e.g., C₆-C₁₂, C₆-C₁₀, or phenyl), or R^{LWW.1}-substituted or unsubstituted heteroarylene (e.g., 5 to 12 membered, 5 to 10 membered, 5 to 9 membered, or 5 to 6 membered). Again, “WW” represents the stated superscript number of the subject L group (1, 2, 3, 1A, 2A, 3A, 1B, 2B, 3B, etc.). R^{LWW.1}, as well as R^{LWW.2} and R^{LWW.3} are as defined above.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present disclosure, whether radioactive or not, are encompassed within the scope of the present disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

“Analog” or “analogue” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

The terms “a” or “an”, as used in herein means one or more. In addition, the phrase “substituted with a[n]”, as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl”, the group may contain one or more unsubstituted C₁-C₂₀ alkyls, and/or one or more unsubstituted 2 to 20 membered heteroalkyls.

Moreover, where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple R¹³ substituents are present, each R¹³ substituent may be distinguished as R^{13A}, R^{13B}, R^{13C}, R^{13D}, etc., wherein each of R^{13A}, R^{13B}, R^{13C}, R^{13D}, etc. is defined within the scope of the definition of R¹³ and optionally differently.

Radioactive substances (e.g., radioisotopes) that may be used as imaging and/or labeling agents in accordance with the embodiments of the disclosure include, but are not limited to, ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y, ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Pb, ²¹²Bi, ²¹³Bi, ²²³Ra, and ²²⁵Ac. Paramagnetic ions that may be used as additional imaging agents in accordance with the embodiments of the disclosure include, but are not limited to, ions of transition and lanthanide metals (e.g., metals having atomic numbers of 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb, and Lu.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The term “leaving group” is used in accordance with its ordinary meaning in chemistry and refers to a moiety (e.g., atom, functional group, molecule) that separates from the molecule following a chemical reaction (e.g., bond formation, reductive elimination, condensation, cross-coupling reaction) involving an atom or chemical moiety to which the leaving group is attached, also referred to herein as the “leaving group reactive moiety”, and a complementary reactive moiety (i.e. a chemical moiety that reacts with the leaving group reactive moiety) to form a new bond between the remnants of the leaving groups reactive moiety and the complementary reactive moiety. Thus, the leaving group reactive moiety and the complementary reactive moiety form a complementary reactive group pair. Non limiting examples of leaving groups include hydrogen, hydroxide, organotin moieties (e.g., organotin heteroalkyl), halogen (e.g., Br), perfluoroalkylsulfonates (e.g. triflate), tosylates, mesylates, water, alcohols, nitrate, phosphate, thioether, amines, ammonia, fluoride, carboxylate, phenoxides, boronic acid, boronate esters, and alkoxides. In embodiments, two molecules with leaving groups are allowed to contact, and upon a reaction and/or bond formation (e.g., acyloin condensation, aldol condensation, Claisen condensation, Stille reaction) the leaving groups separates from the respective molecule.

A person of ordinary skill in the art will understand when a variable (e.g., moiety or linker) of a compound or of a compound genus (e.g., a genus described herein) is described by a name or formula of a standalone compound with all valencies filled, the unfilled valence(s) of the variable will be dictated by the context in which the variable is used. For example, when a variable of a compound as described herein is connected (e.g., bonded) to the remainder of the compound through a single bond, that variable is understood to represent a monovalent form (i.e., capable of forming a single bond due to an unfilled valence) of a standalone compound (e.g., if the variable is named “methane” in an embodiment but the variable is known to be attached by a single bond to the remainder of the compound, a person of ordinary skill in the art would understand that the variable is actually a monovalent form of methane, i.e., methyl or —CH₃). Likewise, for a linker variable (e.g., L¹, L², or L³ as described herein), a person of ordinary skill in the art will understand that the variable is the divalent form of a standalone compound (e.g., if the variable is assigned to “PEG” or “polyethylene glycol” in an embodiment but the variable is connected by two separate bonds to the remainder of the compound, a person of ordinary skill in the art would understand that the variable is a divalent (i.e., capable of forming two bonds through two unfilled valences) form of PEG instead of the standalone compound PEG).

As used herein, the term “salt” refers to acid or base salts of the compounds used in the methods of the present invention. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

Thus, the compounds of the present disclosure may exist as salts. The present disclosure includes such salts. Non-limiting examples of such salts include hydrochlorides, hydrobromides, phosphates, sulfates, methanesulfonates, nitrates, maleates, acetates, citrates, fumarates, proprionates, tartrates (e.g., (+)-tartrates, (−)-tartrates, or mixtures thereof including racemic mixtures), succinates, benzoates, and salts with amino acids such as glutamic acid, and quaternary ammonium salts (e.g., methyl iodide, ethyl iodide, and the like). These salts may be prepared by methods known to those skilled in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value.

“Contacting” is used in accordance with its plain ordinary meaning and refers to the process of allowing at least two distinct species (e.g., chemical compounds including biomolecules or cells) to become sufficiently proximal to react, interact or physically touch. It should be appreciated; however, the resulting reaction product can be produced directly from a reaction between the added reagents or from an intermediate from one or more of the added reagents that can be produced in the reaction mixture.

In this disclosure, “comprises,” “comprising,” “containing” and “having” and the like can have the meaning ascribed to them in U.S. Patent law and can mean “includes,” “including,” and the like. “Consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

The term “polymer” is used in accordance with its plain ordinary meaning in the art, and refers to a molecule including repeating subunits (e.g., polymerized monomers).

The term “dehydrogenated polymer” as used herein refers to a polymer including a carbon-carbon double bond.

The term “olefin metathesis” is used in accordance with its plain ordinary meaning in the art, and refers to an organic reaction involving the redistribution of fragments of alkenes (olefins) by the scission and regeranation of carbon-carbon double bonds.

The term “olefin isomerization” is used in accordance with its plain ordinary meaning in the art, and refers to an organic reaction involving the relocation of a carbon-carbon double bond in a molecule to a different part of the molecule. In embodiments, olefin isomerization is isomerization of a terminal olefin to an internal olefin.

The term “dehydrogenation” is used in accordance with its plain ordinary meaning in the art, and refers to an organic reaction involving the removal of hydrogen, e.g., from an organic molecule. In embodiments, dehydrogenation is the conversion of an alkane to an olefin.

The term “catalyst” is used in accordance with its plain ordinary meaning in the art, and refers to a species that increases the rate of a chemical reaction. The catalyst is not consumed in the reaction and can continue to act repeatedly.

The term “high-density polyethylene” or “HDPE” refers to a thermoplastic polymer produced from the monomer ethylene and is known for its high strength-to-density ratio. Typically, the density of HDPE ranges from about 0.93 g/cm³ to about 0.97 g/cm³. HDPE has minimal branching of its polymer chains and is therefore denser than low-density polyethylene.

The term “low-density polyethylene” or “LDPE” refers to a thermoplastic polymer produced from the monomer ethylene. Typically, the density of LDPE ranges from about 0.917 g/cm³ to about 0.930 g/cm³.

The term “linear low-density polyethylene” or “LLDPE” refers to a substantially linear polyethylene with significant numbers of short branches. LLDPE differs from LDPE because of the absence of long chain branching. Typically, the density of LLDPE ranges from about 0.91 g/cm³ to about 0.94 g/cm³.

### II. Methods

In an aspect is provided a method of degrading a substance, the method including: (i) conducting one or more reaction(s) to form one or more olefin units in the substance; (ii) subjecting, after step (i), to conditions for olefin metathesis; and (iii) subjecting, after step (i), to conditions for olefin isomerization. In embodiments, step (i) includes subjecting the substance to conditions for dehydrogenation.

In an aspect is provided a method of making a substituted or unsubstituted alkene, the method including subjecting a dehydrogenated polymer to conditions for olefin metathesis and to conditions for olefin isomerization.

In an aspect is provided a method of making a substituted or unsubstituted alkene, the method comprising subjecting a first substance to conditions for olefin metathesis and to conditions for olefin isomerization; wherein: (i) the first substance includes at least 20 carbon atoms per molecule; or (ii) prior to subjecting the first substance to conditions for olefin metathesis and to conditions for olefin isomerization, the method includes subjecting a second substance to conditions for dehydrogenation, thereby forming the first substance.

In an aspect is provided a method of making a substituted or unsubstituted alkene, the method comprising subjecting a first substance to conditions for olefin metathesis and to conditions for olefin isomerization; wherein: (i) the first substance includes at most 10% unsaturated alkene monomer units; (ii) the first substance includes at least 20 carbon atoms per molecule; or (iii) prior to subjecting the first substance to conditions for olefin metathesis and to conditions for olefin isomerization, the method includes subjecting a second substance to conditions for dehydrogenation, thereby forming the first substance.

In embodiments, the subjecting to conditions for olefin metathesis and subjecting to conditions for olefin isomerization are conducted simultaneously. In embodiments, the subjecting to conditions for olefin metathesis and subjecting to conditions for olefin isomerization are conducted simultaneously via orthogonal tandem catalysis.

In embodiments, the substance includes one or more polymers. In embodiments, the substance includes one polymer. In embodiments, the substance includes multiple polymers. In embodiments, the substance includes one or more polyolefins. In embodiments, the substance includes one polyolefin. In embodiments, the substance includes multiple polyolefins. In embodiments, the substance is a polyolefin.

In embodiments, the substance includes one or more polymers including one or more monomer units selected from ethylene units, propene units, 1-butene units, 2-butene units, isobutene units, 1-pentene units, 2-pentene units, 1-hexene units, 2-hexene units, 3-hexene units, 1-heptene units, 1-octene units, 1-nonene units, 1-decene units, styrene units, vinyl chloride units, vinyl fluoride units, cyclopentene units, cyclohexene units, cycloheptene units, cyclooctene units, cyclononane units, cyclodecene units, cycloundecene units, cyclododecene, and norbornene units. In embodiments, the substance includes one or more monomer units selected from ethylene, propene, styrene, and vinyl chloride. In embodiments, the substance includes one or more monomer units selected from ethylene and propene. In embodiments, the substance includes one or more monomer units selected from ethylene.

In embodiments, the substance includes one or more co-polymers including one or more monomer units selected from ethylene units, propene units, 1-butene units, 2-butene units, isobutene units, 1-pentene units, 2-pentene units, 1-hexene units, 2-hexene units, 3-hexene units, 1-heptene units, 1-octene units, 1-nonene units, 1-decene units, styrene units, vinyl chloride units, vinyl fluoride units, cyclopentene units, cyclohexene units, cycloheptene units, cyclooctene units, cyclononane units, cyclodecene units, cycloundecene units, cyclododecene, and norbornene units. In embodiments, the substance includes one or more co-polymers including one or more monomer units selected from ethylene units, propene units, 1-butene units, 2-butene units, isobutene units, 1-pentene units, 2-pentene units, 1-hexene units, 2-hexene units, 3-hexene units, 1-heptene units, 1-octene units, 1-nonene units, 1-decene units, styrene units, and vinyl chloride units. In embodiments, the substance includes one or more co-polymers including one or more monomer units selected from ethylene units, propene units, styrene units, and vinyl chloride units. In embodiments, the substance includes one or more co-polymers including one or more monomer units selected from ethylene units and propene units. In embodiments, the substance includes one or more co-polymers including one or more monomer units selected from ethylene units.

In embodiments, the substance includes one or more block co-polymers including one or more blocks selected from polyethylene block(s), polypropylene block(s), poly(vinyl chloride) block(s), polybutene block(s), polyisobutene block(s), poly(1-butene) block(s), poly(2-butene) block(s), polypentene block(s), poly(4-methyl-1-pentene) block(s), polystyrene block(s), poly(cyclopentene) block(s), poly(cyclohexene) block(s), poly(cycloheptene) block(s), poly(cyclooctene) block(s), poly(cyclononene) block(s), poly(cyclodecene) block(s), poly(cycloundecene) block(s), poly(cyclododecene) block(s), and polynorbornene. In embodiments, the substance includes one or more block co-polymers including one or more blocks selected from polyethylene block(s), polypropylene block(s), poly(vinyl chloride) block(s), polyisobutene block(s), poly(4-methyl-1-pentene) block(s), polystyrene block(s), and poly(cyclooctene) block(s). In embodiments, the substance includes one or more block co-polymers including one or more blocks selected from polyethylene block(s), polypropylene block(s), poly(vinyl chloride) block(s), and polystyrene block(s). In embodiments, the substance includes one or more block co-polymers including one or more blocks selected from polyethylene block(s) and polypropylene block(s). In embodiments, the substance includes one or more block co-polymers including one or more blocks selected from polyethylene block(s).

In embodiments, the substance includes one or more homopolymers selected from polyethylene, polypropylene, polystyrene, poly(vinyl chloride), polybutene, polyisobutene, polybutene, poly(1-butene), poly(2-butene), polyisobutylene, polypentene, poly(4-methyl-1-pentene), polystyrene, and poly(vinyl chloride). In embodiments, the substance includes polyethylene. In embodiments, the substance is polyethylene. In embodiments, the substance includes waste polyethylene. In embodiments, the substance includes one or more polyethylenes. In embodiments, the substance is one or more polyethylenes. In embodiments, the substance includes one or more polyethylene(s) selected from high-density polyethylene, low-density polyethylene, linear low-density polyethylene, and co-polymers thereof. In embodiments, the substance includes one or more polyethylene(s) selected from high-density polyethylene, low-density polyethylene, and co-polymers thereof. In embodiments, the substance includes one or more polyethylene(s) selected from high-density polyethylene and low-density polyethylene. In embodiments, the substance includes high-density polyethylene. In embodiments, the substance includes low-density polyethylene. In embodiments, the substance includes linear low-density polyethylene. In embodiments, the substance is high-density polyethylene. In embodiments, the substance is low-density polyethylene. In embodiments, the substance is linear low-density polyethylene.

In embodiments, the substance includes polypropylene. In embodiments, the substance includes syndiotactic polypropylene. In embodiments, the substance includes atactic polypropylene. In embodiments, the substance includes isotactic polypropylene.

In embodiments, the substance includes plastic waste. In embodiments, the substance includes plastic waste selected from milk jugs, shampoo bottles, lawn chairs, food containers, trash bags, barrels, drainage pipes, grocery bags, and cereal box liners.

In embodiments, the formation of one or more olefin units in the substance includes subjecting the substance to conditions for dehydrogenation. In embodiments, the formation of one or more olefin units in the substance includes conducting on the substance one or more dehydrogenation reactions. In embodiments, the one or more dehydrogenation reactions include one or more reactions including removal of one or more proton-hydride pairs, wherein each of the one or more proton-hydride pairs includes one proton and one hydride. In embodiments, the one or more dehydrogenation reactions transform a reactant into a dehydrogenated substance. In embodiments, the one or more dehydrogenation reactions include one or more acceptorless dehydrogenation reactions. In embodiments, the one or more dehydrogenation reactions include one or more transfer dehydrogenation reactions. In embodiments, the one or more dehydrogenation reactions include one or more transfer dehydrogenation reactions that include treatment of the substance with one or more hydrogen acceptors. In embodiments, the one or more dehydrogenation reactions include one or more oxidative dehydrogenation reactions. In embodiments, the one or more dehydrogenation reactions include treatment of the substance with one or more hydrogen acceptors. In embodiments, the one or more dehydrogenation reactions produce a dehydrogenated substance. In embodiments, the dehydrogenated substance is purified. In embodiments, the dehydrogenated substance is purified via one or more techniques selected from distillation or precipitation from one or more organic solvents, or precipitation from water.

In embodiments, the one one or more transfer dehydrogenation reactions include treatment with one or more hydrogen acceptor(s) including: one or more bonds independently selected from C—C bonds(s), C═C bond(s), C≡C bond(s), N—N bond(s), N═N bond(s), N≡N bond(s), O—O bond(s), O═O bond(s), C—N bond(s), C═N bond(s), C≡N bond(s), C—O bond(s), C—O bond(s), C≡O bond(s), S—S bond(s), C—S bond(s), and C═S bond(s); or one or more rings. In embodiments, the one or more hydrogen acceptors include: one or more bonds independently selected from C═C bond(s), C≡C bond(s), N═N bond(s), N≡N bond(s), O—O bond(s), O═O bond(s), C═N bond(s), C≡N bond(s), C═O bond(s), C≡O bond(s), S—S bond(s), and C═S bond(s); or one or more rings. In embodiments, the one or more hydrogen acceptors include: one or more bonds independently selected from C═C bond(s), C≡C bond(s), N═N bond(s), N≡N bond(s), O—O bond(s), O═O bond(s), C═N bond(s), C═O bond(s), C≡O bond(s), S—S bond(s), and C═S bond(s); or one or more rings. In embodiments, the one or more hydrogen acceptors include: one or more bonds independently selected from C═C bond(s), C≡C bond(s), N═N bond(s), O—O bond(s), O═O bond(s), C═N bond(s), C—O bond(s), C≡O bond(s), S—S bond(s), and C═S bond(s); or one or more rings. In embodiments, the one or more hydrogen acceptors include: one or more bonds independently selected from C═C bond(s), C═C bond(s), N═N bond(s), O—O bond(s), O═O bond(s), C═O bond(s), and C≡O bond(s); or one or more rings. In embodiments, the one or more hydrogen acceptors include: one or more bonds independently selected from C═C bond(s), C≡C bond(s), O—O bond(s), O═O bond(s), C═O bond(s), and C≡O bond(s); or one or more rings. In embodiments, the one or more hydrogen acceptors include: one or more bonds independently selected from C═C bond(s), C≡C bond(s), O═O bond(s), C═O bond(s), and C≡O bond(s); or one or more rings. In embodiments, the one or more hydrogen acceptors include: one or more bonds independently selected from C═C bond(s), C≡C bond(s), C═O bond(s), and C≡O bond(s); or one or more rings. In embodiments, the one or more hydrogen acceptors include: one or more bonds independently selected from C═C bond(s) and C≡C bond(s); or one or more rings. In embodiments, the one or more hydrogen acceptors include: one or more bonds independently selected from C═C bond(s) and C≡C bond(s). In embodiments, the one or more hydrogen acceptors include one or more C═C bond(s).

In embodiments, the one or more transfer dehydrogenation reactions include treatment with one or more hydrogen acceptors selected from ethylene, propylene, butene, tert-butylethylene, norbornene, norbornadiene, butadiene, styrene, acetylene, O₂, CO₂, CO, optionally substituted benzoquinone, and N₂. In embodiments, the one or more hydrogen acceptors is interpedently selected from ethylene, propylene, butene, tert-butylethylene, norbornene, norbornadiene, butadiene, and styrene. In embodiments, the one or more hydrogen acceptors is independently selected from ethylene, propylene, tert-butylethylene, norbornene, and norbornadiene. In embodiments, the one or more hydrogen acceptors is independently selected from ethylene, propylene, tert-butylethylene, and norbornene. In embodiments, the one or more hydrogen acceptors is independently selected from ethylene, tert-butylethylene, and norbornene. In embodiments, the one or more hydrogen acceptors is independently selected from tert-butylethylene, and norbornene. In embodiments, the one or more hydrogen acceptors is independently selected from tert-butylethylene and norbornene. In embodiments, the one or more hydrogen acceptors is independently selected from norbornene. In embodiments, the one or more hydrogen acceptors is independently selected from tert-butylethylene. In embodiments, the one or more hydrogen acceptors is ethylene. In embodiments, the one or more hydrogen acceptors is propene.

In embodiments, the conditions for dehydrogenation include conducting one or more reactions at temperatures ranging from 20 to 1000° C. In embodiments, the conditions for dehydrogenation include conducting one or more reactions at temperatures ranging from 50 to 500° C. In embodiments, the conditions for dehydrogenation include conducting one or more reactions at temperatures ranging from 100 to 400° C. In embodiments, the conditions for dehydrogenation include conducting one or more reactions at temperatures ranging from 100 to 300° C. In embodiments, the conditions for dehydrogenation include conducting one or more reactions at temperatures ranging from 100 to 200° C. In embodiments, the dehydrogenation is conducted at a temperature of at least 20° C., at least 50° C., at least 65° C., at least 80° C., at least 100° C., at least 120° C., at least 200° C., at least 250° C., at least 300° C., at least 350° C., at least 400° C., at least 500° C., at least 600° C., at least 1000° C., or more. Alternatively, or in addition, in embodiments, the dehydrogenation is conducted at a temperature of at most 20° C., at most 50° C., at most 65° C., at most 80° C., at most 100° C., at most 120° C., at most 200° C., at most 250° C., at most 300° C., at most 350° C., at most 400° C., at most 500° C., at most 600° C., at most 1000° C., or less.

In embodiments, the conditions for dehydrogenation include conducting one or more reactions at a temperature of about 20° C. to about 1000° C. In embodiments, the conditions for dehydrogenation include conducting one or more reactions at a temperature of at least about 20° C. In embodiments, the conditions for dehydrogenation include conducting one or more reactions at a temperature of at most about 1000° C. In embodiments, the conditions for dehydrogenation include conducting one or more reactions at a temperature of about 20° C. to about 65° C., about 20° C. to about 100° C., about 20° C. to about 150° C., about 20° C. to about 200° C., about 20° C. to about 300° C., about 20° C. to about 350° C., about 20° C. to about 400° C., about 20° C. to about 450° C., about 20° C. to about 500° C., about 20° C. to about 600° C., about 20° C. to about 1000° C., about 65° C. to about 100° C., about 65° C. to about 150° C., about 65° C. to about 200° C., about 65° C. to about 300° C., about 65° C. to about 350° C., about 65° C. to about 400° C., about 65° C. to about 450° C., about 65° C. to about 500° C., about 65° C. to about 600° C., about 65° C. to about 1000° C., about 100° C. to about 150° C., about 100° C. to about 200° C., about 100° C. to about 300° C., about 100° C. to about 350° C., about 100° C. to about 400° C., about 100° C. to about 450° C., about 100° C. to about 500° C., about 100° C. to about 600° C., about 100° C. to about 1,000° C., about 150° C. to about 200° C., about 150° C. to about 300° C., about 150° C. to about 350° C., about 150° C. to about 400° C., about 150° C. to about 450° C., about 150° C. to about 500° C., about 150° C. to about 600° C., about 150° C. to about 1,000° C., about 200° C. to about 300° C., about 200° C. to about 350° C., about 200° C. to about 400° C., about 200° C. to about 450° C., about 200° C. to about 500° C., about 200° C. to about 600° C., about 200° C. to about 1,000° C., about 300° C. to about 350° C., about 300° C. to about 400° C., about 300° C. to about 450° C., about 300° C. to about 500° C., about 300° C. to about 600° C., about 300° C. to about 1000° C., about 350° C. to about 400° C., about 350° C. to about 450° C., about 350° C. to about 500° C., about 350° C. to about 600° C., about 350° C. to about 1,000° C., about 400° C. to about 450° C., about 400° C. to about 500° C., about 400° C. to about 600° C., about 400° C. to about 1000° C., about 450° C. to about 500° C., about 450° C. to about 600° C., about 450° C. to about 1000° C., about 500° C. to about 600° C., about 500° C. to about 1000° C., or about 600° C. to about 1000° C. In embodiments, the conditions for dehydrogenation include conducting one or more reactions at a temperature of about 20° C., about 65° C., about 100° C., about 150° C., about 200° C., about 300° C., about 350° C., about 400° C., about 450° C., about 500° C., about 600° C., or about 1000° C.

In embodiments, the conditions for dehydrogenation include subjecting a neat substance to one or more dehydrogenation reaction(s). In embodiments, the conditions for dehydrogenation include melting the substance. In embodiments, the conditions for dehydrogenation include melting the polymer. In embodiments, the conditions for dehydrogenation include melting the polyethylene.

In embodiments, the conditions for dehydrogenation include contacting a substance with one or more solvents. In embodiments, the conditions for dehydrogenation include contacting a substance with one or more organic solvents. In embodiments, the conditions for dehydrogenation include contacting a substance with one or more hydrocarbon solvents. In embodiments, the conditions for dehydrogenation include contacting a substance with one or more solvents selected from benzene, toluene, o-xylene, m-xylene, p-xylene, dichlorobenzene (e.g., o-dichlorobenzene), trichlorobenzene (e.g., 1,2,4-trichlorobenzene), dioxane, tetrahydrofuran, tetrahydropyran, acetone, chloroform, carbon tetrachloride, dichloromethane, ethyl acetate, pentane, pentanes, hexane, hexanes, heptane, heptanes, octane, octanes, nonane, nonanes, decane, decanes, undecane, undecanes, dodecane, and dodecanes. In embodiments, the conditions for dehydrogenation include contacting a substance with a mixture of multiple organic solvents.

In embodiments, the conditions for dehydrogenation include conducting a batch reaction. In embodiments, the conditions for dehydrogenation include flowing one or more gases over the headspace of a batch reaction. In embodiments, the conditions for dehydrogenation include flowing one or more gases selected from helium, argon, nitrogen, and hydrogen over the headspace of a batch reaction.

In embodiments, the conditions for dehydrogenation include contacting a substance with one or more dehydrogenation catalysts. In embodiments, the one or more dehydrogenation catalysts includes a homogeneous catalyst. In embodiments, the one or more dehydrogenation catalysts includes a heterogeneous catalyst. In embodiments, the one or more dehydrogenation catalysts is (are) immobilized.

In embodiments, the formation of one or more olefin units in the substance includes subjecting the substance to conditions for cracking. In embodiments, the (i) conducting one or more reaction(s) to form one or more olefin units in the substance includes subjecting the substance to conditions for cracking. In embodiments, the cracking is thermal cracking. In embodiments, the cracking is catalytic cracking. In embodiments, the cracking is fluid catalytic cracking. In embodiments, the cracking includes hydrocracking. In embodiments, the conditions for cracking include heating to a temperature of 100° C. to 900° C. In embodiments, the conditions for cracking include heating to a temperature of 300° C. to 700° C. In embodiments, the conditions for cracking include heating to a temperature of 300° C. to 600° C. In embodiments, the conditions for cracking include heating to a temperature of 400° C. to 650° C. In embodiments, the conditions for cracking include treatment with one or more catalysts selected from alumina, silica, zeolite X, and zeolite Y. In embodiments, the conditions for cracking include selectively forming olefins from paraffins. In embodiments, the conditions for cracking include selectively forming monoenes or nonconjugated dienes from paraffins. In embodiments, the conditions for cracking include selectively forming monoenes from paraffins.

In embodiments, the formation of one or more olefin units includes a polymerization. In embodiments, the polymerization includes termination of chain growth. In embodiments, the polymerization includes termination of chain growth to release one or more olefin units. In embodiments, the termination of chain growth includes β-hydride elimination. In embodiments, the termination of chain growth includes chain-transfer to monomer. In embodiments, the formation of one or more olefin units includes a polymerization of one or more monomer units or co-monomer units including two or more degrees of unsaturation. In embodiments, the one or more monomer units co-monomer units including two or more degrees of unsaturation is a polyene (e.g., diene) or alkyne. In embodiments, the one or more monomer units co-monomer units including two or more degrees of unsaturation is selected from butadiene, acetylene, and norbornadiene.

In embodiments, the formation of one or more olefin units includes post-polymerization modification. In embodiments, the the post-polymerization modification includes one or more reaction sequences selected from (i) C—H olefination; (ii) C—H functionalization to install a heteroatom, X, followed by elimination of HX; (iii) CH formylation to install an aldehyde followed by Wittig coupling; (iv) CH carboxylation to install an ester followed by treatment with Tebbe's reagent or Petasis reagent; (v) Julia olefination; and (vi) CH alkynylation to install one or more alkynes followed by reduction of the one or more alkynes to alkenes. In embodiments, the post-polymerization modification includes C—H functionalization to install a heteroatom, X, followed by elimination of HX. In embodiments, the post-polymerization modification includes C—H halogenation, followed by elimination. In embodiments, the post-polymerization modification includes C—H halogenation, followed by base-promoted elimination.

In embodiments, the formation of one or more olefin units includes installation of a leaving group on a substance. In embodiments, the formation of one or more olefin units includes installation of a leaving group on a substance and elimination of the leaving group. In embodiments, the leaving group is selected from —F, —Cl, —Br, —I, —OTf, —OTs, —OMs, —(OR₂)⁺, —(NR₃)⁺, OC(O)R, OC(O)OR, OC(O)X, and SO₂R, wherein R and X are independently selected from H, substituted or unsubstituted C₁-C₃₀ alkyl, and 3 to 10 membered heterocycloalkyl. In embodiments, the leaving group is selected from —F, —Cl, —Br, and —I. In embodiments, the leaving group is selected from —Cl, —Br, and —I. In embodiments, the leaving group is —I.

In embodiments, the method further includes a pre-metathesis step conducted after the (i) forming one or more olefins, before the (ii) subjecting to conditions for olefin metathesis, and before the (iii) subjecting to conditions for olefin isomerization. In embodiments, the pre-metathesis step includes treating with one or more olefin metathesis catalysts. In embodiments, the pre-metathesis step includes treating with one or more olefins. In embodiments, the pre-metathesis step includes treating with ethylene. In embodiments, the pre-metathesis step includes treating with ethylene and one or more olefin metathesis catalysts.

In embodiments, the conditions for olefin metathesis include treatment with one or more olefins. In embodiments, the conditions for olefin metathesis include treatment with one or more exogenously added olefins. In embodiments, the conditions for olefin metathesis include treatment with 0.1-1000 equiv of the one or more olefins. In embodiments, the conditions for olefin metathesis include treatment with one or more olefins independently selected from C₂-C₄₀ olefins. In embodiments, the conditions for olefin metathesis include treatment with an optionally substituted C₂ olefin. In embodiments, the conditions for olefin metathesis include treatment with one or more olefins independently selected from ethylene, propylene, 1-butene, 2-butene, 1-pentene, 2-pentene, 1-hexene, 2-hexene, 3-hexene, 1-heptene, 2-heptene, 3-hepene, 1-octene, 2-octene, 3-octene, 4-octene, 1-nonene, 2-nonene, 3-nonene, 4-nonene, 1-decene, 2-decene, 3-decene, 4-decene, 5-decene, 1-undecene, 2-undecene, 3-undecene, 4-undecene, 5-undecene, 1-dodecene, 2-dodecene, 3-dodecene, 4-dodecene, 5-dodecene, 6-dodecene, 1-tridecene, 2-tridecene, 3-tridecene, 4-tridecene, 5-tridecene, 6-tridecene, 1-tetradecene, 2-tetradecene, 3-tetradecene, 4-tetradecene, 5-tetradecene, 6-tetradecene, and 7-tetradecene.

In embodiments, the one or more olefins include one or more gaseous olefins. In embodiments, the conditions for olefin metathesis include treatment with 0.1-1000 bar of the one or more gaseous olefins. In embodiments, the conditions for olefin metathesis include treatment with 5-100 bar of the one or more gaseous olefins. In embodiments, the conditions for olefin metathesis include treatment with 10-100 bar of the one or more gaseous olefins. In embodiments, the conditions for olefin metathesis include treatment with ethylene. In embodiments, the conditions for olefin metathesis include treatment with ethylene in a pressure of 0.1-1000 bar. In embodiments, the conditions for olefin metathesis include conditions for ethenolysis. In embodiments, the conditions for olefin metathesis include treatment with propylene. In embodiments, the conditions for olefin metathesis include treatment with propylene in a pressure of 0.1-1000 bar. In embodiments, the conditions for olefin metathesis include conditions for propenolysis. In embodiments, the conditions for olefin metathesis include treatment with butenes. In embodiments, the conditions for olefin metathesis include treatment with butenes in a pressure of 0.1-1000 bar. In embodiments, the conditions for olefin metathesis include conditions for butenolysis. In embodiments, the conditions for olefin metathesis include treatment with 2-butene. In embodiments, the conditions for olefin metathesis include treatment with 2-butene in a pressure of 0.1-1000 bar. In embodiments, the conditions for olefin metathesis include conditions for 2-butenolysis. In embodiments, the conditions for olefin metathesis include treatment with ethylene and one or more olefin metathesis catalysts.

In embodiments, the conditions for olefin metathesis include heating at 20-500° C. In embodiments, the conditions for olefin metathesis include heating at 20-200° C. In embodiments, the conditions for olefin metathesis include heating at 40-170° C. In embodiments, the conditions for olefin metathesis include heating at 40-160° C. In embodiments, the conditions for olefin metathesis include heating at 140-170° C. In embodiments, the conditions for olefin metathesis include heating at 40-100° C.

In embodiments, a neat substance is subjected to the conditions for olefin metathesis. In embodiments, the conditions for olefin metathesis include melting the dehydrogenated substance. In embodiments, the conditions for olefin metathesis include melting a polymer. In embodiments, the conditions for olefin metathesis include contacting a substance with one or more solvents. In embodiments, the conditions for olefin metathesis include contacting a substance with one or more organic solvents. In embodiments, the conditions for olefin metathesis include contacting a substance with one or more hydrocarbon solvents. In embodiments, the conditions for olefin metathesis include contacting a substance with one or more solvents selected from benzene, toluene, o-xylene, m-xylene, p-xylene, dichlorobenzene (e.g., o-dichlorobenzene), trichlorobenzene (e.g., 1,2,4-trichlorobenzene), dioxane, tetrahydrofuran, tetrahydropyran, acetone, chloroform, carbon tetrachloride, dichloromethane, ethyl acetate, pentane, pentanes, hexane, hexanes, heptane, heptanes, octane, octanes, nonane, nonanes, decane, decanes, undecane, undecanes, dodecane, and dodecanes.

In embodiments, the conditions for olefin metathesis include conducting a batch reaction. In embodiments, the conditions for olefin metathesis include flowing one or more gases over the headspace of a batch reaction. In embodiments, the conditions for olefin metathesis include conducting a continuous-flow reaction. In embodiments, the conditions for olefin metathesis include flowing one or more gases selected from helium, argon, nitrogen, and hydrogen over the headspace of a batch reaction.

In embodiments, the conditions for olefin metathesis include contacting the substance with one or more olefin metathesis catalysts. In embodiments, the one or more olefin metathesis catalysts include one or more homogeneous catalyst(s). In embodiments, the one or more olefin metathesis catalysts include one or more heterogeneous catalyst(s). In embodiments, one or more of the one or more olefin metathesis catalysts is (are) immobilized. In embodiments, the one or more olefin metathesis catalyst(s) is/are selected from Schrock-type olefin metathesis catalyst(s), Grubbs-type olefin metathesis catalyst(s), Hoveyda/Grubbs-type olefin metathesis catalyst(s), and heterogenous-type olefin metathesis catalyst(s). In embodiments, the one or more olefin metathesis catalysts includes Hoveyda Grubbs II. In embodiments, the one or more olefin metathesis catalysts is Hoveyda Grubbs II. In embodiments, the one or more olefin metathesis catalysts includes Ultra-Nitro-Cat. In embodiments, the one or more olefin metathesis catalysts includes Re₂O₇. In embodiments, the one or more olefin metathesis catalysts includes Re₂O₇ on alumina. In embodiments, the one or more olefin metathesis catalysts includes Re₂O₇ on silica. In embodiments, the one or more olefin metathesis catalysts includes Re₂O₇ on a zeolite. In embodiments, the one or more olefin metathesis catalysts includes Re₂O₇ on zeolite Y. In embodiments, the one or more olefin metathesis catalysts includes tungsten.

In embodiments, the conditions for olefin metathesis include conducting more than one metathesis reaction conducted per molecule present in the substance. In embodiments, the conditions for olefin metathesis include conducting more than one nondegenerate metathesis reaction conducted per molecule present in the substance. In embodiments, the conditions for olefin metathesis include conducting 2-100,000 nondegenerate metathesis reactions per molecule present in the substance.

In embodiments, the conditions for olefin isomerization include conditions for positional olefin isomerization. In embodiments, the conditions for olefin isomerization include conditions for positional, long-range olefin isomerization. In embodiments, the conditions for olefin isomerization include conditions for positional, short-range olefin isomerization. In embodiments, the conditions for olefin isomerization include conditions for isomerization of terminal olefins to internal olefins. In embodiments, the conditions for olefin isomerization include conducting one or more olefin isomerization(s) proceeding through one or more intermediate(s) independently selected from metal-hydride intermediate(s), metal-allyl intermediate(s), cationic intermediate(s), carbocationic intermediate(s), anionic intermediate(s), carbanionic intermediate(s), radical intermediate(s), metal-radical intermediate(s), radical cationic intermediate(s), and radical anionic intermediate(s). In embodiments, the one or more olefin isomerization(s) proceed(s) through one or more metal-hydride intermediate(s). In embodiments, the one or more olefin isomerization(s) proceed(s) through one or more metal-allyl intermediate(s). In embodiments, the one or more olefin isomerization(s) proceed(s) through one or more cationic intermediate(s).

In embodiments, the conditions for olefin isomerization include heating at 20° C. to 1000° C. In embodiments, the conditions for olefin isomerization include heating at 20° C. to 200° C. In embodiments, the conditions for olefin isomerization include heating at 20° C. to 175° C.

In embodiments, a neat substance is subjected to the conditions for olefin isomerization. In embodiments, the conditions for olefin isomerization include melting the dehydrogenated substance. In embodiments, the conditions for olefin isomerization include melting the polymer. In embodiments, the conditions for olefin isomerization include contacting a substance with one or more solvents. In embodiments, the conditions for olefin isomerization include contacting a substance with one or more solvents selected from benzene, toluene, o-xylene, m-xylene, p-xylene, dichlorobenzene (e.g., o-dichlorobenzene), trichlorobenzene (e.g., 1,2,4-trichlorobenzene), dioxane, tetrahydrofuran, tetrahydropyran, acetone, chloroform, carbon tetrachloride, dichloromethane, ethyl acetate, pentane, pentanes, hexane, hexanes, heptane, heptanes, octane, octanes, nonane, nonanes, decane, decanes, undecane, undecanes, dodecane, and dodecanes. In embodiments, the conditions for olefin isomerization include treatment with one or more bases.

In embodiments, the conditions for olefin isomerization include contacting a substance with one or more olefin isomerization catalysts. In embodiments, the one or more olefin isomerization catalysts include a homogeneous catalyst. In embodiments, the one or more olefin isomerization catalysts include a heterogeneous catalyst. In embodiments, the one or more olefin isomerization catalysts include an immobilized catalyst. In embodiments, the conditions for olefin isomerization include treatment with one or more olefin isomerization catalysts. In embodiments, the conditions for olefin isomerization include treatment with one or more homogeneous olefin isomerization catalysts. In embodiments, the conditions for olefin isomerization include treatment with one or more heterogeneous olefin isomerization catalysts.

In embodiments, the conditions for olefin isomerization include conducting a batch reaction. In embodiments, the conditions for olefin isomerization include flowing one or more gases over the headspace of a batch reaction. In embodiments, the conditions for olefin isomerization include conducting a continuous-flow reaction. In embodiments, the conditions for olefin isomerization include flowing one or more gases selected from helium, argon, nitrogen, and hydrogen over the headspace of a batch reaction.

In embodiments, the (b) subjecting to conditions for olefin metathesis and the (c) subjecting to conditions for olefin isomerization are collectively conducted as an isomerizing olefin metathesis. In embodiments, the one or more olefins formed in the (a) forming one or more olefins is subjected to an isomerizing olefin metathesis. In embodiments, the isomerizing olefin metathesis includes an isomerizing ethenolysis. In embodiments, the isomerizing olefin metathesis is an isomerizing ethenolysis. In embodiments, the isomerizing olefin metathesis includes repetition of a cycle including (i) olefin metathesis and (ii) olefin isomerization. In embodiments, the cycle including (i) olefin metathesis and (ii) olefin isomerization is repeated 1-500,000 times. In embodiments, the cycle including (i) olefin metathesis and (ii) olefin isomerization is repeated 1-200,000 times. In embodiments, the cycle including (i) olefin metathesis and (ii) olefin isomerization is repeated 1-100,000 times. In embodiments, the cycle including (i) olefin metathesis and (ii) olefin isomerization is repeated 1-50,000 times. In embodiments, the cycle including (i) olefin metathesis and (ii) olefin isomerization is repeated 1-5,000 times. In embodiments, the cycle including (i) olefin metathesis and (ii) olefin isomerization is repeated 500 times. In embodiments, the cycle including (i) olefin metathesis and (ii) olefin isomerization is repeated 1-50 times. In embodiments, the cycle including (i) olefin metathesis and (ii) olefin isomerization is repeated at least 1 time, at least 50 times, at least 100 times, at least 500 times, at least 2000 times, at least 10,000 times, at least 50,000 times, at least 100,000 times, at least 500,000 or more. Alternatively, or in addition, in embodiments, the cycle including (i) olefin metathesis and (ii) olefin isomerization is repeated at most 1 time, at most 50 times, at most 100 times, at most 500 times, at most 2000 times, at most 10,000 times, at most 50,000 times, at most 100,000 times, at most 500,000 or less.

In embodiments, the cycle including (i) olefin metathesis and (ii) olefin isomerization is repeated about 1 time to about 500,000 times. In embodiments, the cycle including (i) olefin metathesis and (ii) olefin isomerization is repeated at least about 1 time. In embodiments, the cycle including (i) olefin metathesis and (ii) olefin isomerization is repeated at most about 500,000 times. In embodiments, the cycle including (i) olefin metathesis and (ii) olefin isomerization is repeated about 1 time to about 50 times, about 1 time to about 100 times, about 1 time to about 500 times, about 1 time to about 1,000 times, about 1 time to about 5,000 times, about 1 time to about 10,000 times, about 1 time to about 25,000 times, about 1 time to about 50,000 times, about 1 time to about 100,000 times, about 1 time to about 250,000 times, about 1 time to about 500,000 times, about 50 times to about 100 times, about 50 times to about 500 times, about 50 times to about 1,000 times, about 50 times to about 5,000 times, about 50 times to about 10,000 times, about 50 times to about 25,000 times, about 50 times to about 50,000 times, about 50 times to about 100,000 times, about 50 times to about 250,000 times, about 50 times to about 500,000 times, about 100 times to about 500 times, about 100 times to about 1,000 times, about 100 times to about 5,000 times, about 100 times to about 10,000 times, about 100 times to about 25,000 times, about 100 times to about 50,000 times, about 100 times to about 100,000 times, about 100 times to about 250,000 times, about 100 times to about 500,000 times, about 500 times to about 1,000 times, about 500 times to about 5,000 times, about 500 times to about 10,000 times, about 500 times to about 25,000 times, about 500 times to about 50,000 times, about 500 times to about 100,000 times, about 500 times to about 250,000 times, about 500 times to about 500,000 times, about 1,000 times to about 5,000 times, about 1,000 times to about 10,000 times, about 1,000 times to about 25,000 times, about 1,000 times to about 50,000 times, about 1,000 times to about 100,000 times, about 1,000 times to about 250,000 times, about 1,000 times to about 500,000 times, about 5,000 times to about 10,000 times, about 5,000 times to about 25,000 times, about 5,000 times to about 50,000 times, about 5,000 times to about 100,000 times, about 5,000 times to about 250,000 times, about 5,000 times to about 500,000 times, about 10,000 times to about 25,000 times, about 10,000 times to about 50,000 times, about 10,000 times to about 100,000 times, about 10,000 times to about 250,000 times, about 10,000 times to about 500,000 times, about 25,000 times to about 50,000 times, about 25,000 times to about 100,000 times, about 25,000 times to about 250,000 times, about 25,000 times to about 500,000 times, about 50,000 times to about 100,000 times, about 50,000 times to about 250,000 times, about 50,000 times to about 500,000 times, about 100,000 times to about 250,000 times, about 100,000 times to about 500,000 times, or about 250,000 times to about 500,000 times. In embodiments, the cycle including (i) olefin metathesis and (ii) olefin isomerization is repeated about 1 time, about 50 times, about 100 times, about 500 times, about 1,000 times, about 5,000 times, about 10,000 times, about 25,000 times, about 50,000 times, about 100,000 times, about 250,000 times, or about 500,000 times.

In embodiments, the isomerizing olefin metathesis is conducted at 20-500° C. In embodiments, a neat substance is subjected to the conditions for isomerizing olefin metathesis. In embodiments, the conditions for isomerizing olefin metathesis include melting the dehydrogenated substance. In embodiments, the conditions for isomerizing olefin metathesis include melting a polymer. In embodiments, the conditions for isomerizing olefin metathesis include contacting a substance with one or more solvents. In embodiments, the conditions for isomerizing olefin metathesis include contacting a substance with one or more solvents selected from benzene, toluene, o-xylene, m-xylene, p-xylene, dichlorobenzene (e.g., o-dichlorobenzene), trichlorobenzene (e.g., 1,2,4-trichlorobenzene), dioxane, tetrahydrofuran, tetrahydropyran, acetone, chloroform, carbon tetrachloride, dichloromethane, ethyl acetate, pentane, pentanes, hexane, hexanes, heptane, heptanes, octane, octanes, nonane, nonanes, decane, decanes, undecane, undecanes, dodecane, dodecanes. In embodiments, the conditions for isomerizing olefin metathesis include treatment with one or more bases.

In embodiments, the conditions for isomerizing olefin metathesis include contacting a substance with one or more olefin metathesis catalysts. In embodiments, one or more of the one or more olefin metathesis catalysts is capable of olefin isomerization. In embodiments, the conditions for isomerizing olefin metathesis include contacting a substance with one or more olefin isomerization catalysts. In embodiments, one or more of the one or more olefin isomerization catalysts is capable of catalyzing olefin metathesis. In embodiments, the conditions for isomerizing olefin metathesis include contacting a substance with one or more olefin metathesis catalysts and with one or more olefin isomerization catalysts.

In embodiments, the conditions for isomerizing olefin metathesis include conducting a batch reaction. In embodiments, the conditions for isomerizing olefin metathesis include flowing one or more gases over the headspace of a batch reaction. In embodiments, the conditions for isomerizing olefin metathesis include conducting a continuous-flow reaction. In embodiments, the conditions for isomerizing olefin metathesis include flowing one or more gases selected from helium, argon, nitrogen, and hydrogen over the headspace of a batch reaction. In embodiments, the conditions for isomerizing olefin metathesis include continuously removing one or more gases. In embodiments, the conditions for isomerizing olefin metathesis include continuously removing propene. In embodiments, the conditions for isomerizing olefin metathesis include continuously adding one or more gases. In embodiments, the conditions for isomerizing olefin metathesis include continuously adding ethylene.

In embodiments, the method produces olefins. In embodiments, the method produces olefins that include fewer carbons per molecule than the substance. In embodiments, the method further includes reactive distillation. In embodiments, the method produces propene. In embodiments, the method produces propene in a yield of 0.1 to 100%. In embodiments, the method produces propene in a yield of 50% to 100%. In embodiments, the method produces propene in a yield of 50% to 99.9%. In embodiments, the method produces propene in a yield of 50% to 99%. In embodiments, the method produces propene in a yield of 60% to 95%. In embodiments, the method produces propene in a selectivity of 0% to 100%. In embodiments, the method produces propene in a selectivity of 0.01% to 99.99%. In embodiments, the method produces propene in a selectivity of 1% to 99.99%. In embodiments, the method produces propene in a selectivity of 50% to 99.99%.

In an aspect is provided a method of making an alkene, the method including mixing a dehydrogenated polymer with one or more olefin metathesis reagent(s) and one or more isomerization reagent(s); wherein: (i) the dehydrogenated polymer includes at least 20 carbon atoms; or (ii) prior to the mixing, the method includes mixing a polymer with one or more dehydrogenating reagent(s), thereby forming the dehydrogenated polymer.

In an aspect is provided a method of making an alkene, the method including mixing a dehydrogenated polymer with one or more olefin metathesis reagent(s) and one or more isomerization reagent(s); wherein: (i) the dehydrogenated polymer includes at most 10% unsaturated alkene monomer units; (ii) the dehydrogenated polymer includes at least 20 carbon atoms; or (iii) prior to the mixing, the method includes mixing a polymer with one or more dehydrogenating reagent(s), thereby forming the dehydrogenated polymer.

In an aspect is provided a method of making a dehydrogenated polymer, the method including mixing a polymer with one or more dehydrogenating reagent(s), wherein: (i) a percentage of from 0.01% to 1.5% or from 1.7% to 2.3% or from 2.4% to 4.3% or from 4.5% to 13.2% of monomer units in the dehydrogenated polymer are unsaturated; (ii) the dehydrogenated polymer has a number average molecular weight of about 2,200 Da or greater; (iii) the dehydrogenated polymer has a number average molecular weight of about 2,200 Da to about 6,500 Da and a percentage of 0.01% to 10% of monomer units are unsaturated; or (iv) the dehydrogenated polymer has a number average molecular weight of about 1,000 Da to about 500,000 Da and a percentage of from 0.01% to from 1.5% or from 1.7% to 2.3% or from 2.4% to 4.3% or from 4.5% to 10% of monomer units in the dehydrogenated polymer are unsaturated.

In embodiments, the substituted or unsubstituted alkene is a substituted or unsubstituted propene. In embodiments, the first substance is a substituted or unsubstituted dehydrogenated polyethylene.

In embodiments, the the dehydrogenated polymer has a number average molecular weight of from about 2,200 Da to about 6,500 Da or from about 6,700 Da to about 15,000 Da. In embodiments, the dehydrogenated polymer has a number average molecular weight of from about 2,200 Da to about 6,500 Da. In embodiments, the dehydrogenated polymer has a number average molecular weight of from about 2,200 Da to about 6,000 Da. In embodiments, the dehydrogenated polymer has a number average molecular weight of from about 2,200 Da to about 5,000 Da. In embodiments, the dehydrogenated polymer has a number average molecular weight of from about 2,200 Da to about 4,000 Da. In embodiments, the dehydrogenated polymer has a number average molecular weight of from about 2,200 Da to about 4,000 Da. In embodiments, the dehydrogenated polymer has a number average molecular weight of from about 6,700 Da to about 500,000 Da. In embodiments, the dehydrogenated polymer has a number average molecular weight of from about 6,700 Da to about 400,000 Da. In embodiments, the dehydrogenated polymer has a number average molecular weight of from about 6,700 Da to about 300,000 Da. In embodiments, the dehydrogenated polymer has a number average molecular weight of from about 6,700 Da to about 200,000 Da. In embodiments, the dehydrogenated polymer has a number average molecular weight of from about 6,700 Da to about 100,000 Da. In embodiments, the dehydrogenated polymer has a number average molecular weight of from about 6,700 Da to about 80,000 Da. In embodiments, the dehydrogenated polymer has a number average molecular weight of from about 6,700 Da to about 60,000 Da. In embodiments, the dehydrogenated polymer has a number average molecular weight of from about 6,700 Da to about 40,000 Da. In embodiments, the dehydrogenated polymer has a number average molecular weight of from about 6,700 Da to about 20,000 Da. In embodiments, the dehydrogenated polymer has a number average molecular weight of from about 6,700 Da to about 15,000 Da.

In embodiments, the the dehydrogenated polymer has a number average molecular weight of from 2,200 Da to 6,500 Da or from 6,700 Da to 15,000 Da. In embodiments, the dehydrogenated polymer has a number average molecular weight of from 2,200 Da to 6,500 Da. In embodiments, the dehydrogenated polymer has a number average molecular weight of from 2,200 Da to 6,000 Da. In embodiments, the dehydrogenated polymer has a number average molecular weight of from 2,200 Da to 5,000 Da. In embodiments, the dehydrogenated polymer has a number average molecular weight of from 2,200 Da to 4,000 Da. In embodiments, the dehydrogenated polymer has a number average molecular weight of from 2,200 Da to 4,000 Da. In embodiments, the dehydrogenated polymer has a number average molecular weight of from 6,700 Da to 500,000 Da. In embodiments, the dehydrogenated polymer has a number average molecular weight of from 6,700 Da to 400,000 Da. In embodiments, the dehydrogenated polymer has a number average molecular weight of from 6,700 Da to 300,000 Da. In embodiments, the dehydrogenated polymer has a number average molecular weight of from 6,700 Da to 200,000 Da. In embodiments, the dehydrogenated polymer has a number average molecular weight of from 6,700 Da to 100,000 Da. In embodiments, the dehydrogenated polymer has a number average molecular weight of from 6,700 Da to 80,000 Da. In embodiments, the dehydrogenated polymer has a number average molecular weight of from 6,700 Da to 60,000 Da. In embodiments, the dehydrogenated polymer has a number average molecular weight of from 6,700 Da to 40,000 Da. In embodiments, the dehydrogenated polymer has a number average molecular weight of from 6,700 Da to 20,000 Da. In embodiments, the dehydrogenated polymer has a number average molecular weight of from 6,700 Da to 15,000 Da.

In embodiments, a percentage of from about 10% or less of monomer units in the dehydrogenated polymer are unsaturated. In embodiments, a percentage of from about 0.01% to about 11% of monomer units in the dehydrogenated polymer are unsaturated. In embodiments, a percentage of from about 0.01% to about 10% of monomer units in the dehydrogenated polymer are unsaturated. In embodiments, a percentage of from about 0.01% to about 9% of monomer units in the dehydrogenated polymer are unsaturated. In embodiments, a percentage of from about 0.01% to about 8% of monomer units in the dehydrogenated polymer are unsaturated. In embodiments, a percentage of from about 0.01% to about 1.5% of monomer units in the dehydrogenated polymer are unsaturated. In embodiments, a percentage of from about 2.4% to about 4.3% of monomer units in the dehydrogenated polymer are unsaturated. In embodiments, a percentage of from about 4.5% to about 13.2% of monomer units in the dehydrogenated polymer are unsaturated. In embodiments, a percentage of from about 1% to about 3% of monomer units in the dehydrogenated polymer are unsaturated. In embodiments, a percentage of from about 1.5% to about 3% of monomer units in the dehydrogenated polymer are unsaturated. In embodiments, a percentage of from about 1.7% to about 2.3% of monomer units in the dehydrogenated polymer are unsaturated. In embodiments, a percentage of from about 1.7% to about 2.1% of monomer units in the dehydrogenated polymer are unsaturated. In embodiments, a percentage of from about 1.9% to about 2.1% of monomer units in the dehydrogenated polymer are unsaturated. In embodiments, a percentage of about 1.7% of monomer units in the dehydrogenated polymer are unsaturated. In embodiments, a percentage of about 1.8% of monomer units in the dehydrogenated polymer are unsaturated. In embodiments, a percentage of about 1.9% of monomer units in the dehydrogenated polymer are unsaturated. In embodiments, a percentage of about 2% of monomer units in the dehydrogenated polymer are unsaturated. In embodiments, a percentage of about 2.1% of monomer units in the dehydrogenated polymer are unsaturated. In embodiments, a percentage of about 2.2% of monomer units in the dehydrogenated polymer are unsaturated. In embodiments, a percentage of about 2.3% of monomer units in the dehydrogenated polymer are unsaturated.

In embodiments, a percentage of 10% or less of monomer units in the dehydrogenated polymer are unsaturated. In embodiments, a percentage of from 0.01% to 11% of monomer units in the dehydrogenated polymer are unsaturated. In embodiments, a percentage of from 0.01% to 10% of monomer units in the dehydrogenated polymer are unsaturated. In embodiments, a percentage of from 0.01% to 9% of monomer units in the dehydrogenated polymer are unsaturated. In embodiments, a percentage of from 0.01% to 8% of monomer units in the dehydrogenated polymer are unsaturated. In embodiments, a percentage of from 0.01% to 1.5% of monomer units in the dehydrogenated polymer are unsaturated. In embodiments, a percentage of from 2.4% to 4.3% of monomer units in the dehydrogenated polymer are unsaturated. In embodiments, a percentage of from 4.5% to 13.2% of monomer units in the dehydrogenated polymer are unsaturated. In embodiments, a percentage of from 1% to 3% of monomer units in the dehydrogenated polymer are unsaturated. In embodiments, a percentage of from 1.5% to 3% of monomer units in the dehydrogenated polymer are unsaturated. In embodiments, a percentage of from 1.7% to 2.3% of monomer units in the dehydrogenated polymer are unsaturated. In embodiments, a percentage of from 1.7% to 2.1% of monomer units in the dehydrogenated polymer are unsaturated. In embodiments, a percentage of from 1.9% to 2.1% of monomer units in the dehydrogenated polymer are unsaturated. In embodiments, a percentage of 1.7% of monomer units in the dehydrogenated polymer are unsaturated. In embodiments, a percentage of 1.8% of monomer units in the dehydrogenated polymer are unsaturated. In embodiments, a percentage of 1.9% of monomer units in the dehydrogenated polymer are unsaturated. In embodiments, a percentage of 2% of monomer units in the dehydrogenated polymer are unsaturated. In embodiments, a percentage of 2.1% of monomer units in the dehydrogenated polymer are unsaturated. In embodiments, a percentage of 2.2% of monomer units in the dehydrogenated polymer are unsaturated. In embodiments, a percentage of 2.3% of monomer units in the dehydrogenated polymer are unsaturated.

In embodiments, the dehydrogenated polymer has a number average molecular weight of about 1,000 Da to about 500,000 Da and a percentage of from 0.01% to 1.5% or from 1.7% to 2.3% or from 2.4% to 4.3% or from 4.5% to 10% of monomer units in the dehydrogenated polymer are unsaturated. In embodiments, the dehydrogenated polymer has a number average molecular weight of about 2,200 Da to about 100,000 Da and a percentage of from 0.01% to 1.5% or from 1.7% to 2.3% or from 2.4% to 4.3% or from 4.5% to 10% of monomer units in the dehydrogenated polymer are unsaturated. In embodiments, the dehydrogenated polymer has a number average molecular weight of about 2,200 Da to about 50,000 Da and a percentage of from 0.01% to 1.5% or from 1.7% to 2.3% or from 2.4% to 4.3% or from 4.5% to 10% of monomer units in the dehydrogenated polymer are unsaturated. In embodiments, the dehydrogenated polymer has a number average molecular weight of about 2,200 Da to about 20,000 Da and a percentage of from 0.01% to 1.5% or from 1.7% to 2.3% or from 2.4% to 4.3% or from 4.5% to 10% of monomer units in the dehydrogenated polymer are unsaturated. In embodiments, the dehydrogenated polymer has a number average molecular weight of about 2,200 Da to about 10,000 Da and a percentage of from 0.01% to 1.5% or from 1.7% to 2.3% or from 2.4% to 4.3% or from 4.5% to 10% of monomer units in the dehydrogenated polymer are unsaturated. In embodiments, the dehydrogenated polymer has a number average molecular weight of about 2,200 Da to about 6,500 Da and a percentage of from 0.01% to 1.5% or from 1.7% to 2.3% or from 2.4% to 4.3% or from 4.5% to 10% of monomer units in the dehydrogenated polymer are unsaturated.

In embodiments, the method further includes mixing the dehydrogenated polymer with one or more olefin metathesis reagent(s) and one or more isomerization reagent(s), thereby forming an alkene.

In embodiments, the dehydrogenated polymer is a substituted or unsubstituted dehydrogenated polyethylene. In embodiments, the alkene is a substituted or unsubstituted propene.

In embodiments, the dehydrogenated polymer, the one or more olefin metathesis reagent(s), and the one or more isomerization reagent(s) are mixed simultaneously. In embodiments, the dehydrogenated polymer and the one or more olefin metathesis reagent(s) are mixed prior to adding the one or more isomerization reagent(s). In embodiments, the dehydrogenated polymer and one or more additional olefin metathesis reagent(s) are mixed prior to adding the one or more olefin metathesis reagent(s) and the one or more isomerization reagent(s).

In embodiments, the one or more olefin metathesis reagent(s) includes an ethenolysis catalyst. In embodiments, the one or more olefin metathesis reagent(s) includes one or more homogeneous olefin metathesis catalyst(s) or one or more heterogeneous olefin metathesis catalyst(s). In embodiments, the one or more olefin metathesis reagent(s) includes one or more homogeneous olefin metathesis catalyst(s). In embodiments, the one or more olefin metathesis reagent(s) includes one or more heterogeneous olefin metathesis catalyst(s). In embodiments, the one or more olefin metathesis reagent(s) further includes ethylene.

In embodiments, the homogeneous olefin metathesis catalyst is

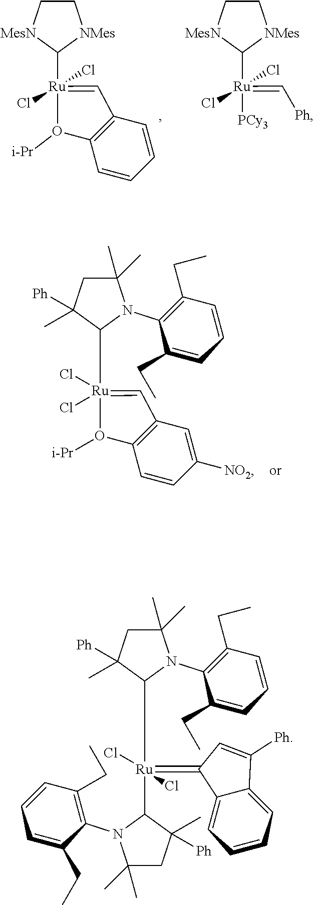

In embodiments, the homogeneous olefin metathesis catalyst is

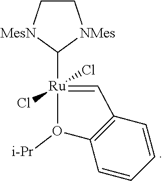

In embodiments, the homogeneous olefin metathesis catalyst is

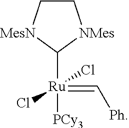

In embodiments, the homogeneous olefin metathesis catalyst is

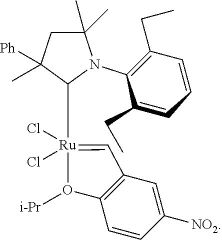

In embodiments, the homogeneous olefin metathesis catalyst is

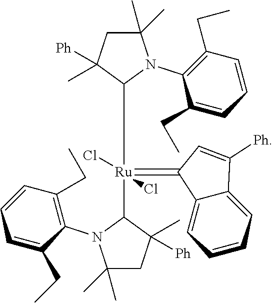

In embodiments, the heterogeneous olefin metathesis catalyst is Re₂O₇/Al₂O₃ or WO₃/Al₂O₃. In embodiments, the heterogeneous olefin metathesis catalyst is Re₂O₇/Al₂O₃. In embodiments, the heterogeneous olefin metathesis catalyst is WO₃/Al₂O₃.

In embodiments, the one or more isomerization reagent(s) includes one or more homogeneous isomerization catalyst(s) or one or more heterogeneous isomerization catalyst(s). In embodiments, the one or more isomerization reagent(s) includes one or more homogeneous isomerization catalyst(s). In embodiments, the one or more isomerization reagent(s) includes one or more heterogeneous isomerization catalyst(s).

In embodiments, the one or more homogeneous isomerization catalyst(s) is selected from:

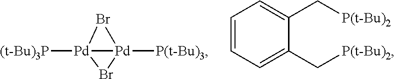

[Rh(cod)₂]BF₄ and

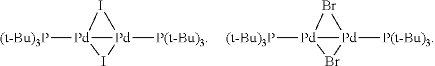

In embodiments, the one or more homogeneous isomerization catalyst(s) is
In embodiments, the one or more homogeneous isomerization catalyst(s) is

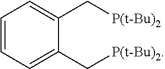

In embodiments, the one or more homogeneous isomerization catalyst(s) is [Rh(cod)₂]BF₄. In embodiments, the one or more homogeneous isomerization catalyst(s) is

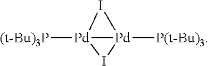

In embodiments, the one or more heterogeneous isomerization catalyst(s) is selected from: Na/Al₂O₃, K/Al₂O₃, Mg/Al₂O₃, MgO, NaO₂, and KO₂. In embodiments, the one or more heterogeneous isomerization catalyst(s) is Na/Al₂O₃. In embodiments, the one or more heterogeneous isomerization catalyst(s) is K/Al₂O₃. In embodiments, the one or more heterogeneous isomerization catalyst(s) is Mg/Al₂O₃. In embodiments, the one or more heterogeneous isomerization catalyst(s) is MgO. In embodiments, the one or more heterogeneous isomerization catalyst(s) is NaO₂. In embodiments, the one or more heterogeneous isomerization catalyst(s) is KO₂.

In embodiments, the one or more heterogeneous isomerization catalyst includes a Group VI or Group VII metal (W, Mo, Re) on SiO₂ or Al₂O₃. In embodiments, Group VI or Group VII metal (W, Mo, Re) on SiO₂ or Al₂O₃ is first treated with a mild base (e.g., Na₂CO₃). Without being bound to any particularly theory, in embodiments the mild base is sufficient to neutralize acidic sites. In embodiments, the one or more heterogeneous isomerization catalyst includes K, Na, Na/K alloy, or Li supported on Al₂O₃. In embodiments, the one or more heterogeneous isomerization catalyst includes includes an alkali metal (e.g., a Group VI or Group VII metal such as W, Mo, Re) and a SiO₂ support. In embodiments, the one or more heterogeneous isomerization catalyst includes K, Na, Na/K alloy, or Li supported on SiO₂.

In embodiments, the one or more heterogeneous metathesis catalyst further includes a tin promoter. In embodiments, the tin promotor is a tetraalkyl tin promotor. In embodiments, the one or more heterogeneous metathesis catalyst further includes a tetramethyl tin promoter. In embodiments, the one or more metathesis isomerization catalyst further includes a tetraethyl tin promoter. In embodiments, the one or more heterogeneous metathesis catalyst further includes a tetrapropyl tin promoter. In embodiments, the one or more heterogeneous metathesis catalyst further includes a tetrabutyl tin promoter. In embodiments, the one or more heterogeneous metathesis catalyst further includes a tetrabenzyl tin promoter.

In embodiments, the substituted or unsubstituted polymer is a high-density polyethylene, a low-density polyethylene, or a linear low-density polyethylene, or a co-polymer thereof. In embodiments, the substituted or unsubstituted polymer is a high-density polyethylene. In embodiments, the substituted or unsubstituted polymer is a low-density polyethylene. In embodiments, the substituted or unsubstituted polymer is a linear low-density polyethylene.

In embodiments, the one or more dehydrogenating reagent(s) includes a transfer dehydrogenation catalyst.

In embodiments, the one or more dehydrogenating reagent(s) includes a hydrogen acceptor. In embodiments, the hydrogen acceptor includes a C—C bond, a C═C bond, a C≡C bond, an N—N bond, an N═N bond, an N≡N bond, an O—O bond, an O—O bond, a C—N bond, a C═N bond, a C≡N bond, a C—O bond, a C═O bond, a C≡O bond, an S—S bond, a C—S bond, or a C═S bond. In embodiments, the hydrogen acceptor includes a C—C bond. In embodiments, the hydrogen acceptor includes a C═C bond. In embodiments, the hydrogen acceptor includes a C≡C bond. In embodiments, the hydrogen acceptor includes an N—N bond. In embodiments, the hydrogen acceptor includes an N═N bond. In embodiments, the hydrogen acceptor includes an N≡N bond. In embodiments, the hydrogen acceptor includes an O—O bond. In embodiments, the hydrogen acceptor includes an O—O bond. In embodiments, the hydrogen acceptor includes a C—N bond. In embodiments, the hydrogen acceptor includes a C═N bond. In embodiments, the hydrogen acceptor includes a C≡N bond. In embodiments, the hydrogen acceptor includes a C—O bond. In embodiments, the hydrogen acceptor includes a C═O bond. In embodiments, the hydrogen acceptor includes a C≡O bond. In embodiments, the hydrogen acceptor includes an S—S bond. In embodiments, the hydrogen acceptor includes a C—S bond. In embodiments, the hydrogen acceptor includes a C═S bond.

In embodiments, the hydrogen acceptor is ethylene, propylene, butene, tert-butylethylene, norbornene, norbornadiene, butadiene, styrene, acetylene, O₂, CO₂, CO, substituted or unsubstituted benzoquinone, or N₂. In embodiments, the hydrogen acceptor is ethylene. In embodiments, the hydrogen acceptor is propylene. In embodiments, the hydrogen acceptor is butane. In embodiments, the hydrogen acceptor is tert-butylethylene. In embodiments, the hydrogen acceptor is norbornene. In embodiments, the hydrogen acceptor is norbornadiene. In embodiments, the hydrogen acceptor is butadiene. In embodiments, the hydrogen acceptor is styrene. In embodiments, the hydrogen acceptor is acetylene. In embodiments, the hydrogen acceptor is O₂. In embodiments, the hydrogen acceptor is CO₂. In embodiments, the hydrogen acceptor is CO. In embodiments, the hydrogen acceptor is substituted or unsubstituted benzoquinone. In embodiments, the hydrogen acceptor is N₂.

In embodiments, the one or more dehydrogenating reagent(s) includes a heterogeneous dehydrogenating catalyst. In embodiments, the heterogeneous dehydrogenating catalyst includes a plurality of metallic elements. In embodiments, the heterogeneous dehydrogenating catalyst is a bimetallic catalyst. In embodiments, the heterogeneous dehydrogenating catalyst is a Pt/Sn catalyst or a Pt/Zn catalyst. In embodiments, the heterogeneous dehydrogenating catalyst is a Pt/Sn catalyst. In embodiments, the heterogeneous dehydrogenating catalyst is a Pt/Zn catalyst. In embodiments, the heterogeneous dehydrogenating catalyst is Pt/Sn—Al₂O₃. In embodiments, the heterogeneous dehydrogenating catalyst is Pt/Zn—SiO₂.

In embodiments, the one or more dehydrogenating reagent(s) includes a homogeneous dehydrogenating catalyst. In embodiments, the homogeneous dehydrogenating catalyst includes an iridium catalyst. In embodiments, the homogeneous dehydrogenating catalyst includes a rhodium catalyst. In embodiments, the homogeneous dehydrogenating catalyst includes a ruthenium catalyst.

In embodiments, the one or more dehydrogenating reagent(s) includes (R)-(−)-5,5′-Bis[di(3,5-di-tert-butyl-4-methoxyphenyl)phosphino]-4,4′-bi-1,3-benzodioxole, [(4R)-(4,4′-bi-1,3-benzodioxole)-5,5′-diyl]bis[bis(3,5-di-tert-butyl-4-methoxyphenyl)phosphine], also commonly referred to as (R)-DTBM-SEGPHOS. In embodiments, the one or more dehydrogenating reagent(s) includes (R)-(−)-5,5′-Bis[di(3,5-di-tert-butyl-4-methoxyphenyl)phosphino]-4,4′-bi-1,3-benzodioxole, [(4R)-(4,4′-bi-1,3-benzodioxole)-5,5′-diyl]bis[bis(3,5-di-tert-butyl-4-methoxyphenyl)phosphine] and an iridium catalyst.

In embodiments, the homogeneous dehydrogenating catalyst has the formula:

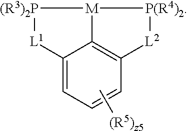

M is Ir, Rh, Ru, or Os.

L¹ and L² are independently —O—, —S—, —NR⁶—, or substituted or unsubstituted alkylene (e.g., C₁-C₈, C₁-C₆, C₁-C₄, or C₁-C₂).

Each R³ and R⁴ is independently substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, or 2 to 3 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R⁵ is independently halogen, —CX⁵₃, —CHX⁵₂, —CH₂X⁵, —OCX⁵₃, —OCH₂X⁵, —OCHX⁵₂, —CN, —SOₙ₅R^{5D}, —SOᵥ₅NR^{5A}R^{5B}, —NR^{5C}NR^{5A}R^{5B}, —ONR^{5A}R^{5B}, —NR^{5C}C(O)NR^{5A}R^{5B}, —N(O)ₘ₅, —NR^{5A}R^{5B}, —C(O)R^{5C}, —C(O)OR^{5C}, —OC(O)R^{5C}, —OC(O)OR^{5C}, —C(O)NR^{5A}R^{5B}, —OC(O)NR^{5A}R^{5B}, —OR^{5D}, —SR^{5D}, —NR^{5A}SO₂R^{5D}, —NR^{5A}C(O)R^{5C}, —NR^{5A}C(O)OR^{5C}, —NR^{5A}OR^{5C}, —P(O)₂(OR^{5A}), —OP(O)₂(OR^{5A}), —OP(R^{5A}) (R^{5B}), —BR^{5A}R^{5B}, —SiR^{5A}R^{5B}R^{5C}, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, or 2 to 3 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

Each R⁶ is independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

Each R^{5A}, R^{5B}, R^{5C}, and R^{5D} is independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, or 2 to 3 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); or R^{5A} and R^{5B} substituents bonded to the same nitrogen atom are joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered).

Each X⁵ is independently —Cl, —Br, —I, or —F.

The symbol n5 is 0, 1, 2, 3, or 4.

Each m5 and v5 is independently 1 or 2.

The symbol z5 is 0, 1, 2, or 3.

In embodiments, the homogeneous dehydrogenating catalyst has the formula:

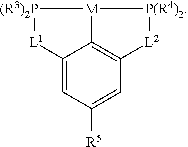

M, L¹, L², R³, R⁴, and R⁵ are as described herein, including in embodiments.

In embodiments, M is Ir, Rh, or Ru. In embodiments, M is Ir. In embodiments, Mis Rh. In embodiments, M is Ru. In embodiments, M is Os.

In embodiments, a substituted L¹ (e.g., substituted alkylene) is substituted with at least one substituent group, size-limited substituent group, or lower substituent group; wherein if the substituted L¹ is substituted with a plurality of groups selected from substituent groups, size-limited substituent groups, and lower substituent groups; each substituent group, size-limited substituent group, and/or lower substituent group may optionally be different. In embodiments, when L¹ is substituted, it is substituted with at least one substituent group. In embodiments, when L¹ is substituted, it is substituted with at least one size-limited substituent group. In embodiments, when L¹ is substituted, it is substituted with at least one lower substituent group.

In embodiments, L¹ is —O—, —S—, —NR⁶—, or unsubstituted C₁-C₁₀ alkylene. In embodiments, L¹ is —O—. In embodiments, L¹ is —S—. In embodiments, L¹ is —NR⁶—. In embodiments, L¹ is —NH—. In embodiments, L¹ is unsubstituted C₁-C₁₀ alkylene. In embodiments, L¹ is unsubstituted methylene. In embodiments, L¹ is unsubstituted ethylene. In embodiments, L¹ is unsubstituted propylene. In embodiments, L¹ is unsubstituted n-propylene. In embodiments, L¹ is unsubstituted isopropylene. In embodiments, L¹ is unsubstituted butylene. In embodiments, L¹ is unsubstituted n-butylene. In embodiments, L¹ is unsubstituted isobutylene. In embodiments, L¹ is unsubstituted tert-butylene. In embodiments, L¹ is substituted C₁-C₁₀ alkylene. In embodiments, L¹ is substituted methylene. In embodiments, L¹ is substituted ethylene. In embodiments, L¹ is substituted propylene. In embodiments, L¹ is substituted n-propylene. In embodiments, L¹ is substituted isopropylene. In embodiments, L¹ is substituted butylene. In embodiments, L¹ is substituted n-butylene. In embodiments, L¹ is substituted isobutylene. In embodiments, L¹ is substituted tert-butylene. In embodiments, the substituted alkylene is substituted with a substituent group.

In embodiments, a substituted L² (e.g., substituted alkylene) is substituted with at least one substituent group, size-limited substituent group, or lower substituent group; wherein if the substituted L² is substituted with a plurality of groups selected from substituent groups, size-limited substituent groups, and lower substituent groups; each substituent group, size-limited substituent group, and/or lower substituent group may optionally be different. In embodiments, when L² is substituted, it is substituted with at least one substituent group. In embodiments, when L² is substituted, it is substituted with at least one size-limited substituent group. In embodiments, when L² is substituted, it is substituted with at least one lower substituent group.

In embodiments, L² is —O—, —S—, —NR⁶—, or unsubstituted C₁-C₁₀ alkylene. In embodiments, L² is —O—. In embodiments, L² is —S—. In embodiments, L² is —NR⁶—. In embodiments, L² is —NH—. In embodiments, L² is unsubstituted C₁-C₁₀ alkylene. In embodiments, L² is unsubstituted methylene. In embodiments, L² is unsubstituted ethylene. In embodiments, L² is unsubstituted propylene. In embodiments, L² is unsubstituted n-propylene. In embodiments, L² is unsubstituted isopropylene. In embodiments, L² is unsubstituted butylene. In embodiments, L² is unsubstituted n-butylene. In embodiments, L² is unsubstituted isobutylene. In embodiments, L² is unsubstituted tert-butylene. In embodiments, L² is substituted C₁-C₁₀ alkylene. In embodiments, L² is substituted methylene. In embodiments, L² is substituted ethylene. In embodiments, L² is substituted propylene. In embodiments, L² is substituted n-propylene. In embodiments, L² is substituted isopropylene. In embodiments, L² is substituted butylene. In embodiments, L² is substituted n-butylene. In embodiments, L² is substituted isobutylene. In embodiments, L² is substituted tert-butylene. In embodiments, the substituted alkylene is substituted with a substituent group.

In embodiments, a substituted R³ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R³ is substituted with a plurality of groups selected from substituent groups, size-limited substituent groups, and lower substituent groups; each substituent group, size-limited substituent group, and/or lower substituent group may optionally be different. In embodiments, when R³ is substituted, it is substituted with at least one substituent group. In embodiments, when R³ is substituted, it is substituted with at least one size-limited substituent group. In embodiments, when R³ is substituted, it is substituted with at least one lower substituent group.

In embodiments, R³ is unsubstituted C₁-C₁₀ alkyl, unsubstituted 2 to 10 membered heteroalkyl, unsubstituted C₃-C₁₀ cycloalkyl, or unsubstituted phenyl. In embodiments, R³ is unsubstituted C₁-C₁₀ alkyl. In embodiments, R³ is unsubstituted methyl. In embodiments, R³ is unsubstituted ethyl. In embodiments, R³ is unsubstituted propyl. In embodiments, R³ is unsubstituted n-propyl. In embodiments, R³ is unsubstituted isopropyl. In embodiments, R³ is unsubstituted butyl. In embodiments, R³ is unsubstituted n-butyl. In embodiments, R³ is unsubstituted isobutyl. In embodiments, R³ is unsubstituted tert-butyl. In embodiments, R³ is unsubstituted neopentyl. In embodiments, R³ is unsubstituted 2 to 10 membered heteroalkyl. In embodiments, R³ is unsubstituted methoxy. In embodiments, R³ is unsubstituted ethoxy. In embodiments, R³ is unsubstituted propoxy. In embodiments, R³ is independently n-propoxy. In embodiments, R³ is unsubstituted isopropoxy. In embodiments, R³ is unsubstituted butoxy. In embodiments, R³ is unsubstituted n-butoxy. In embodiments, R³ is unsubstituted isobutoxy. In embodiments, R³ is unsubstituted tert-butoxy. In embodiments, R³ is unsubstituted C₃-C₁₀ cycloalkyl. In embodiments, R³ is unsubstituted adamantyl. In embodiments, R³ is unsubstituted 1-adamantyl. In embodiments, R³ is unsubstituted 2-adamantyl. In embodiments, R³ is unsubstituted phenyl.

In embodiments, a substituted R⁴ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁴ is substituted with a plurality of groups selected from substituent groups, size-limited substituent groups, and lower substituent groups; each substituent group, size-limited substituent group, and/or lower substituent group may optionally be different. In embodiments, when R⁴ is substituted, it is substituted with at least one substituent group. In embodiments, when R⁴ is substituted, it is substituted with at least one size-limited substituent group. In embodiments, when R⁴ is substituted, it is substituted with at least one lower substituent group.

In embodiments, R⁴ is unsubstituted C₁-C₁₀ alkyl, unsubstituted 2 to 10 membered heteroalkyl, unsubstituted C₃-C₁₀ cycloalkyl, or unsubstituted phenyl. In embodiments, R⁴ is unsubstituted C₁-C₁₀ alkyl. In embodiments, R⁴ is unsubstituted methyl. In embodiments, R⁴ is unsubstituted ethyl. In embodiments, R⁴ is unsubstituted propyl. In embodiments, R⁴ is unsubstituted n-propyl. In embodiments, R⁴ is unsubstituted isopropyl. In embodiments, R⁴ is unsubstituted butyl. In embodiments, R⁴ is unsubstituted n-butyl. In embodiments, R⁴ is unsubstituted isobutyl. In embodiments, R⁴ is unsubstituted tert-butyl. In embodiments, R⁴ is unsubstituted neopentyl. In embodiments, R⁴ is unsubstituted 2 to 10 membered heteroalkyl. In embodiments, R⁴ is unsubstituted methoxy. In embodiments, R⁴ is unsubstituted ethoxy. In embodiments, R⁴ is unsubstituted propoxy. In embodiments, R⁴ is independently n-propoxy. In embodiments, R⁴ is unsubstituted isopropoxy. In embodiments, R⁴ is unsubstituted butoxy. In embodiments, R⁴ is unsubstituted n-butoxy. In embodiments, R⁴ is unsubstituted isobutoxy. In embodiments, R⁴ is unsubstituted tert-butoxy. In embodiments, R⁴ is unsubstituted C₃-C₁₀ cycloalkyl. In embodiments, R⁴ is unsubstituted adamantyl. In embodiments, R⁴ is unsubstituted 1-adamantyl. In embodiments, R⁴ is unsubstituted 2-adamantyl. In embodiments, R⁴ is unsubstituted phenyl.

In embodiments, a substituted R⁵ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁵ is substituted with a plurality of groups selected from substituent groups, size-limited substituent groups, and lower substituent groups; each substituent group, size-limited substituent group, and/or lower substituent group may optionally be different. In embodiments, when R⁵ is substituted, it is substituted with at least one substituent group. In embodiments, when R⁵ is substituted, it is substituted with at least one size-limited substituent group. In embodiments, when R⁵ is substituted, it is substituted with at least one lower substituent group.

In embodiments, R⁵ is independently halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 5 membered, or 5 to 6 membered).

In embodiments, R⁵ is independently halogen. In embodiments, R⁵ is independently —F. In embodiments, R⁵ is independently —Cl. In embodiments, R⁵ is independently-Br. In embodiments, R⁵ is independently —I. In embodiments, R⁵ is independently —CCl₃. In embodiments, R⁵ is independently —CBr₃. In embodiments, R⁵ is independently —CF₃. In embodiments, R⁵ is independently —CI₃. In embodiments, R⁵ is independently —CH₂Cl. In embodiments, R⁵ is independently —CH₂Br. In embodiments, R⁵ is independently —CH₂F. In embodiments, R⁵ is independently —CH₂I. In embodiments, R⁵ is independently —CHCl₂. In embodiments, R⁵ is independently —CHBr₂. In embodiments, R⁵ is independently —CHF₂. In embodiments, R⁵ is independently —CHI₂. In embodiments, R⁵ is independently —CN. In embodiments, R⁵ is independently —OR^{5D}. In embodiments, R⁵ is independently —OCH₃. In embodiments, R⁵ is independently —OH. In embodiments, R⁵ is independently —NH₂. In embodiments, R⁵ is independently —COOH. In embodiments, R⁵ is independently —CONH₂. In embodiments, R⁵ is independently —NO₂. In embodiments, R⁵ is independently —SH. In embodiments, R⁵ is independently —SO₃H. In embodiments, R⁵ is independently —OSO₃H. In embodiments, R⁵ is independently —SO₂NH₂. In embodiments, R⁵ is independently —NHNH₂. In embodiments, R⁵ is independently —ONH₂. In embodiments, R⁵ is independently —NHC(O)NHNH₂. In embodiments, R⁵ is independently —NHC(O)NH₂. In embodiments, R⁵ is independently —NHSO₂H. In embodiments, R⁵ is independently —NHC(O)H. In embodiments, R⁵ is independently —NHC(O)OH. In embodiments, R⁵ is independently —NHOH. In embodiments, R⁵ is independently —OCCl₃. In embodiments, R⁵ is independently —OCBr₃. In embodiments, R⁵ is independently —OCF₃. In embodiments, R⁵ is independently —OCI₃. In embodiments, R⁵ is independently —OCH₂Cl. In embodiments, R⁵ is independently —OCH₂Br. In embodiments, R⁵ is independently —OCH₂F. In embodiments, R⁵ is independently —OCH₂I. In embodiments, R⁵ is independently —OCHCl₂. In embodiments, R⁵ is independently —OCHBr₂. In embodiments, R⁵ is independently —OCHF₂. In embodiments, R⁵ is independently —OCHI₂. In embodiments, R⁵ is independently —OP(R^{5A}) (R^{5B}). In embodiments, R⁵ is independently —OP(C(CH₃)₃)₂. In embodiments, R⁵ is independently unsubstituted C₁-C₁₀ alkyl. In embodiments, R⁵ is independently unsubstituted methyl. In embodiments, R⁵ is independently unsubstituted ethyl. In embodiments, R⁵ is independently unsubstituted propyl. In embodiments, R⁵ is independently unsubstituted n-propyl. In embodiments, R⁵ is independently unsubstituted isopropyl. In embodiments, R⁵ is independently unsubstituted butyl. In embodiments, R⁵ is independently unsubstituted n-butyl. In embodiments, R⁵ is independently unsubstituted isobutyl. In embodiments, R⁵ is independently unsubstituted tert-butyl. In embodiments, R⁵ is independently unsubstituted 2 to 4 membered heteroalkyl. In embodiments, R⁵ is independently unsubstituted methoxy. In embodiments, R⁵ is independently unsubstituted ethoxy. In embodiments, R⁵ is independently unsubstituted propoxy. In embodiments, R⁵ is independently unsubstituted n-propoxy. In embodiments, R⁵ is independently unsubstituted isopropoxy. In embodiments, R⁵ is independently unsubstituted butoxy. In embodiments, R⁵ is independently unsubstituted n-butoxy. In embodiments, R⁵ is independently unsubstituted isobutoxy. In embodiments, R⁵ is independently unsubstituted tert-butoxy.

In embodiments, a substituted R^{5A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{5A} is substituted with a plurality of groups selected from substituent groups, size-limited substituent groups, and lower substituent groups; each substituent group, size-limited substituent group, and/or lower substituent group may optionally be different. In embodiments, when R^{5A} is substituted, it is substituted with at least one substituent group. In embodiments, when R^{5A} is substituted, it is substituted with at least one size-limited substituent group. In embodiments, when R^{5A} is substituted, it is substituted with at least one lower substituent group.

In embodiments, a substituted R^{5B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{5B} is substituted with a plurality of groups selected from substituent groups, size-limited substituent groups, and lower substituent groups; each substituent group, size-limited substituent group, and/or lower substituent group may optionally be different. In embodiments, when R^{5B} is substituted, it is substituted with at least one substituent group. In embodiments, when R^{5B} is substituted, it is substituted with at least one size-limited substituent group. In embodiments, when R^{5B} is substituted, it is substituted with at least one lower substituent group.

In embodiments, a substituted ring formed when R^{5A} and R^{5B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{5A} and R^{5B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{5A} and R^{5B} substituents bonded to the same nitrogen atom are joined is substituted, it is substituted with at least one substituent group. In embodiments, when the substituted ring formed when RA and R^{5B} substituents bonded to the same nitrogen atom are joined is substituted, it is substituted with at least one size-limited substituent group. In embodiments, when the substituted ring formed when R^{5A} and R^{5B} substituents bonded to the same nitrogen atom are joined is substituted, it is substituted with at least one lower substituent group.

In embodiments, a substituted R^{5C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{5C} is substituted with a plurality of groups selected from substituent groups, size-limited substituent groups, and lower substituent groups; each substituent group, size-limited substituent group, and/or lower substituent group may optionally be different. In embodiments, when R^{5C} is substituted, it is substituted with at least one substituent group. In embodiments, when R^{5C} is substituted, it is substituted with at least one size-limited substituent group. In embodiments, when R^{5C} is substituted, it is substituted with at least one lower substituent group.

In embodiments, a substituted R^{5D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{5D} is substituted with a plurality of groups selected from substituent groups, size-limited substituent groups, and lower substituent groups; each substituent group, size-limited substituent group, and/or lower substituent group may optionally be different. In embodiments, when R^{5D} is substituted, it is substituted with at least one substituent group. In embodiments, when R^{5D} is substituted, it is substituted with at least one size-limited substituent group. In embodiments, when R^{5D} is substituted, it is substituted with at least one lower substituent group.

In embodiments, R^{5A} is independently hydrogen or unsubstituted C₁-C₄ alkyl. In embodiments, R^{5A} is independently hydrogen. In embodiments, R^{5A} is independently unsubstituted C₁-C₄ alkyl. In embodiments, R^{5A} is independently unsubstituted methyl. In embodiments, R^{5A} is independently unsubstituted ethyl. In embodiments, R^{5A} is independently unsubstituted propyl. In embodiments, R^{5A} is independently unsubstituted n-propyl. In embodiments, R^{5A} is independently unsubstituted isopropyl. In embodiments, R^{5A} is independently unsubstituted butyl. In embodiments, R^{5A} is independently unsubstituted n-butyl. In embodiments, R^{5A} is independently unsubstituted isobutyl. In embodiments, R^{5A} is independently unsubstituted tert-butyl.

In embodiments, R^{5B} is independently hydrogen or unsubstituted C₁-C₄ alkyl. In embodiments, R^{5B} is independently hydrogen. In embodiments, R^{5B} is independently unsubstituted C₁-C₄ alkyl. In embodiments, R^{5B} is independently unsubstituted methyl. In embodiments, R^{5B} is independently unsubstituted ethyl. In embodiments, R^{5B} is independently unsubstituted propyl. In embodiments, R^{5B} is independently unsubstituted n-propyl. In embodiments, R^{5B} is independently unsubstituted isopropyl. In embodiments, R^{5B} is independently unsubstituted butyl. In embodiments, R^{5B} is independently unsubstituted n-butyl. In embodiments, R^{5B} is independently unsubstituted isobutyl. In embodiments, R^{5B} is independently unsubstituted tert-butyl.

In embodiments, R^{5C} is independently hydrogen or unsubstituted C₁-C₄ alkyl. In embodiments, R^{5C} is independently hydrogen. In embodiments, R^{5C} is independently unsubstituted C₁-C₄ alkyl. In embodiments, R^{5C} is independently unsubstituted methyl. In embodiments, R^{5C} is independently unsubstituted ethyl. In embodiments, R^{5C} is independently unsubstituted propyl. In embodiments, R^{5C} is independently unsubstituted n-propyl. In embodiments, R^{5C} is independently unsubstituted isopropyl. In embodiments, R^{5C} is independently unsubstituted butyl. In embodiments, R^{5C} is independently unsubstituted n-butyl. In embodiments, R^{5C} is independently unsubstituted isobutyl. In embodiments, R^{5C} is independently unsubstituted tert-butyl.

In embodiments, R^{5D} is independently hydrogen or unsubstituted C₁-C₄ alkyl. In embodiments, R^{5D} is independently hydrogen. In embodiments, R^{5D} is independently unsubstituted C₁-C₄ alkyl. In embodiments, R^{5D} is independently unsubstituted methyl. In embodiments, R^{5D} is independently unsubstituted ethyl. In embodiments, R^{5D} is independently unsubstituted propyl. In embodiments, R^{5D} is independently unsubstituted n-propyl. In embodiments, R^{5D} is independently unsubstituted isopropyl. In embodiments, R^{5D} is independently unsubstituted butyl. In embodiments, R^{5D} is independently unsubstituted n-butyl. In embodiments, R^{5D} is independently unsubstituted isobutyl. In embodiments, R^{5D} is independently unsubstituted tert-butyl.

In embodiments, z5 is 0. In embodiments, z5 is 1. In embodiments, z5 is 2. In embodiments, z5 is 3.

In embodiments, a substituted R⁶ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁶ is substituted with a plurality of groups selected from substituent groups, size-limited substituent groups, and lower substituent groups; each substituent group, size-limited substituent group, and/or lower substituent group may optionally be different. In embodiments, when R⁶ is substituted, it is substituted with at least one substituent group. In embodiments, when R⁶ is substituted, it is substituted with at least one size-limited substituent group. In embodiments, when R⁶ is substituted, it is substituted with at least one lower substituent group.

In embodiments, R⁶ is independently hydrogen or unsubstituted C₁-C₄ alkyl. In embodiments, R⁶ is independently hydrogen. In embodiments, R⁶ is independently unsubstituted C₁-C₄ alkyl. In embodiments, R⁶ is independently unsubstituted methyl. In embodiments, R⁶ is independently unsubstituted ethyl. In embodiments, R⁶ is independently unsubstituted propyl. In embodiments, R⁶ is independently unsubstituted n-propyl. In embodiments, R⁶ is independently unsubstituted isopropyl. In embodiments, R⁶ is independently unsubstituted butyl. In embodiments, R⁶ is independently unsubstituted n-butyl. In embodiments, R⁶ is independently unsubstituted isobutyl. In embodiments, R⁶ is independently unsubstituted tert-butyl.

In embodiments, the homogeneous dehydrogenating catalyst is

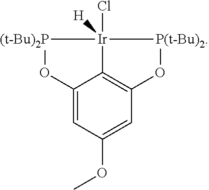

In embodiments, the dehydrogenated polymer includes at least one —C(R¹)═C(R²)— monomer unit.

R¹ is independently hydrogen, halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NR^{1C}C(O)NR^{1A}R^{1B}, —N(O)ₘ₁, —NR^{1A}R^{1B}, —C(O)R^{1C}, —C(O)OR^{1C}, —OC(O)R^{1C}, —OC(O)OR^{1C}, —C(O)NR^{1A}R^{1B}, —OC(O)NR^{1A}R^{1B}, —OR^{1D}, —SR^{1D}, —NR^{1A}SO₂R^{1D}, —NR^{1A}C(O)R^{1C}, —NR^{1A}C(O)OR^{1C}, —NR^{1A}OR^{1C}, —P(O)₂(OR^{1A}), —OP(O)₂(OR^{1A}), —BR^{1A}R^{1B}, —SiR^{1A}R^{1B}R^{1C}, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, or 2 to 3 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R² is independently hydrogen, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NR^{2C}C(O)NR^{2A}R^{2B}, —N(O)ₘ₂, —NR^{2A}R^{2B}, —C(O)R^{2C}, —C(O)OR^{2C}, —OC(O)R^{2C}, —OC(O)OR^{2C}, —C(O)NR^{2A}R^{2B}, —OC(O)NR^{2A}R^{2B}, —OR^{2D}, —SR^{2D}, —NR^{2A}SO₂R^{2D}, —NR^{2A}C(O)R^{2C}, —NR^{2A}C(O)OR^{2C}, —NR^{2A}OR^{2C}, —P(O)₂(OR^{2A}), —OP(O)₂(OR^{2A}), —BR^{2A}R^{2B}, —SiR^{2A}R^{2B}R^{2C}, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, or 2 to 3 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

Each R^{1A}, R^{1B}, R^{1C}, R^{1D}, R^{2A}, R^{2B}, R^{2C}, and R^{2D} is independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, or 2 to 3 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); or R^{1A} and R^{1B} substituents bonded to the same nitrogen atom are joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered); or R^{2A} and R^{2B} substituents bonded to the same nitrogen atom are joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered).

Each X¹ and X² is independently —Cl, —Br, —I, or —F.

Each n1 and n2 is independently 0, 1, 2, 3, or 4.

Each m1, m2, v1, and v2 is independently 1 or 2.

In embodiments, a substituted R¹ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹ is substituted with a plurality of groups selected from substituent groups, size-limited substituent groups, and lower substituent groups; each substituent group, size-limited substituent group, and/or lower substituent group may optionally be different. In embodiments, when R¹ is substituted, it is substituted with at least one substituent group. In embodiments, when R¹ is substituted, it is substituted with at least one size-limited substituent group. In embodiments, when R¹ is substituted, it is substituted with at least one lower substituent group.

In embodiments, R¹ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, or 2 to 3 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, R¹ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, or substituted or unsubstituted C₁-C₂₀ alkyl.

In embodiments, R¹ is independently hydrogen. In embodiments, R¹ is independently substituted or unsubstituted C₁-C₁₀₀₀ alkyl. In embodiments, R¹ is independently substituted or unsubstituted C₁-C₅₀₀ alkyl. In embodiments, R¹ is independently substituted or unsubstituted C₁-C₁₀₀ alkyl. In embodiments, R¹ is independently substituted or unsubstituted C₁-C₅₀ alkyl. In embodiments, R¹ is independently substituted or unsubstituted C₁-C₂₀ alkyl. In embodiments, R¹ is independently substituted or unsubstituted C₁-C₁₀ alkyl. In embodiments, R¹ is independently substituted or unsubstituted C₁-C₅ alkyl. In embodiments, R¹ is independently unsubstituted methyl. In embodiments, R¹ is independently unsubstituted ethyl. In embodiments, R¹ is independently unsubstituted propyl. In embodiments, R¹ is independently unsubstituted n-propyl. In embodiments, R¹ is independently unsubstituted isopropyl. In embodiments, R¹ is independently unsubstituted butyl. In embodiments, R¹ is independently unsubstituted n-butyl. In embodiments, R¹ is independently unsubstituted tert-butyl.

In embodiments, a substituted R^{1A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1A} is substituted with a plurality of groups selected from substituent groups, size-limited substituent groups, and lower substituent groups; each substituent group, size-limited substituent group, and/or lower substituent group may optionally be different. In embodiments, when R^{1A} is substituted, it is substituted with at least one substituent group. In embodiments, when R^{1A} is substituted, it is substituted with at least one size-limited substituent group. In embodiments, when R^{1A} is substituted, it is substituted with at least one lower substituent group.

In embodiments, a substituted R^{1B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1B} is substituted with a plurality of groups selected from substituent groups, size-limited substituent groups, and lower substituent groups; each substituent group, size-limited substituent group, and/or lower substituent group may optionally be different. In embodiments, when R^{1B} is substituted, it is substituted with at least one substituent group. In embodiments, when R^{1B} is substituted, it is substituted with at least one size-limited substituent group. In embodiments, when R^{1B} is substituted, it is substituted with at least one lower substituent group.

In embodiments, a substituted ring formed when R^{1A} and R^{1B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{1A} and R^{1B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{1A} and R^{1B} substituents bonded to the same nitrogen atom are joined is substituted, it is substituted with at least one substituent group. In embodiments, when the substituted ring formed when R^{1A} and R^{1B} substituents bonded to the same nitrogen atom are joined is substituted, it is substituted with at least one size-limited substituent group. In embodiments, when the substituted ring formed when R^{1A} and R^{1B} substituents bonded to the same nitrogen atom are joined is substituted, it is substituted with at least one lower substituent group.

In embodiments, a substituted R^{1C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1C} is substituted with a plurality of groups selected from substituent groups, size-limited substituent groups, and lower substituent groups; each substituent group, size-limited substituent group, and/or lower substituent group may optionally be different. In embodiments, when R^{1C} is substituted, it is substituted with at least one substituent group. In embodiments, when R^{1C} is substituted, it is substituted with at least one size-limited substituent group. In embodiments, when R^{1C} is substituted, it is substituted with at least one lower substituent group.

In embodiments, a substituted R^{1D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1D} is substituted with a plurality of groups selected from substituent groups, size-limited substituent groups, and lower substituent groups; each substituent group, size-limited substituent group, and/or lower substituent group may optionally be different. In embodiments, when R^{1D} is substituted, it is substituted with at least one substituent group. In embodiments, when R^{1D} is substituted, it is substituted with at least one size-limited substituent group. In embodiments, when R^{1D} is substituted, it is substituted with at least one lower substituent group.

In embodiments, R^{1A} is independently hydrogen. In embodiments, R^{1A} is independently substituted or unsubstituted C₁-C₅ alkyl. In embodiments, R^{1A} is independently unsubstituted methyl. In embodiments, R^{1A} is independently unsubstituted ethyl. In embodiments, R^{1A} is independently unsubstituted propyl. In embodiments, R^{1A} is independently unsubstituted n-propyl. In embodiments, R^{1A} is independently unsubstituted isopropyl. In embodiments, R^{1A} is independently unsubstituted butyl. In embodiments, R^{1A} is independently unsubstituted n-butyl. In embodiments, R^{1A} is independently unsubstituted tert-butyl.

In embodiments, R^{1B} is independently hydrogen. In embodiments, R^{1B} is independently substituted or unsubstituted C₁-C₅ alkyl. In embodiments, R^{1B} is independently unsubstituted methyl. In embodiments, R^{1B} is independently unsubstituted ethyl. In embodiments, R^{1B} is independently unsubstituted propyl. In embodiments, R^{1B} is independently unsubstituted n-propyl. In embodiments, R^{1B} is independently unsubstituted isopropyl. In embodiments, R^{1B} is independently unsubstituted butyl. In embodiments, R^{1B} is independently unsubstituted n-butyl. In embodiments, R^{1B} is independently unsubstituted tert-butyl.

In embodiments, R^{1C} is independently hydrogen. In embodiments, R^{1C} is independently substituted or unsubstituted C₁-C₅ alkyl. In embodiments, R^{1C} is independently unsubstituted methyl. In embodiments, R^{1C} is independently unsubstituted ethyl. In embodiments, R^{1C} is independently unsubstituted propyl. In embodiments, R^{1C} is independently unsubstituted n-propyl. In embodiments, R^{1C} is independently unsubstituted isopropyl. In embodiments, R^{1C} is independently unsubstituted butyl. In embodiments, R^{1C} is independently unsubstituted n-butyl. In embodiments, R^{1C} is independently unsubstituted tert-butyl.

In embodiments, R^{1D} is independently hydrogen. In embodiments, R^{1D} is independently substituted or unsubstituted C₁-C₅ alkyl. In embodiments, R^{1D} is independently unsubstituted methyl. In embodiments, R^{1D} is independently unsubstituted ethyl. In embodiments, R^{1D} is independently unsubstituted propyl. In embodiments, R^{1D} is independently unsubstituted n-propyl. In embodiments, R^{1D} is independently unsubstituted isopropyl. In embodiments, R^{1D} is independently unsubstituted butyl. In embodiments, R^{1D} is independently unsubstituted n-butyl. In embodiments, R^{1D} is independently unsubstituted tert-butyl.

In embodiments, a substituted R² (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R² is substituted with a plurality of groups selected from substituent groups, size-limited substituent groups, and lower substituent groups; each substituent group, size-limited substituent group, and/or lower substituent group may optionally be different. In embodiments, when R² is substituted, it is substituted with at least one substituent group. In embodiments, when R² is substituted, it is substituted with at least one size-limited substituent group. In embodiments, when R² is substituted, it is substituted with at least one lower substituent group.

In embodiments, R² is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, or 2 to 3 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, R² is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, or substituted or unsubstituted C₁-C₂₀ alkyl.

In embodiments, R² is independently hydrogen. In embodiments, R² is independently substituted or unsubstituted C₁-C₁₀₀₀ alkyl. In embodiments, R² is independently substituted or unsubstituted C₁-C₅₀₀ alkyl. In embodiments, R² is independently substituted or unsubstituted C₁-C₁₀₀ alkyl. In embodiments, R² is independently substituted or unsubstituted C₁-C₅₀ alkyl. In embodiments, R² is independently substituted or unsubstituted C₁-C₂₀ alkyl. In embodiments, R² is independently substituted or unsubstituted C₁-C₁₀ alkyl. In embodiments, R² is independently substituted or unsubstituted C₁-C₅ alkyl. In embodiments, R² is independently unsubstituted methyl. In embodiments, R² is independently unsubstituted ethyl. In embodiments, R² is independently unsubstituted propyl. In embodiments, R² is independently unsubstituted n-propyl. In embodiments, R² is independently unsubstituted isopropyl. In embodiments, R² is independently unsubstituted butyl. In embodiments, R² is independently unsubstituted n-butyl. In embodiments, R² is independently unsubstituted tert-butyl.

In embodiments, a substituted R^{2A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2A} is substituted with a plurality of groups selected from substituent groups, size-limited substituent groups, and lower substituent groups; each substituent group, size-limited substituent group, and/or lower substituent group may optionally be different. In embodiments, when R^{2A} is substituted, it is substituted with at least one substituent group. In embodiments, when R^{2A} is substituted, it is substituted with at least one size-limited substituent group. In embodiments, when R^{2A} is substituted, it is substituted with at least one lower substituent group.

In embodiments, a substituted R^{2B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2B} is substituted with a plurality of groups selected from substituent groups, size-limited substituent groups, and lower substituent groups; each substituent group, size-limited substituent group, and/or lower substituent group may optionally be different. In embodiments, when R^{2B} is substituted, it is substituted with at least one substituent group. In embodiments, when R^{2B} is substituted, it is substituted with at least one size-limited substituent group. In embodiments, when R^{2B} is substituted, it is substituted with at least one lower substituent group.

In embodiments, a substituted ring formed when R^{2A} and R^{2B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{2A} and R^{2B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{2A} and R^{2B} substituents bonded to the same nitrogen atom are joined is substituted, it is substituted with at least one substituent group. In embodiments, when the substituted ring formed when R^{2A} and R^{2B} substituents bonded to the same nitrogen atom are joined is substituted, it is substituted with at least one size-limited substituent group. In embodiments, when the substituted ring formed when R^{2A} and R^{2B} substituents bonded to the same nitrogen atom are joined is substituted, it is substituted with at least one lower substituent group.

In embodiments, a substituted R^{2C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2C} is substituted with a plurality of groups selected from substituent groups, size-limited substituent groups, and lower substituent groups; each substituent group, size-limited substituent group, and/or lower substituent group may optionally be different. In embodiments, when R^{2C} is substituted, it is substituted with at least one substituent group. In embodiments, when R^{2C} is substituted, it is substituted with at least one size-limited substituent group. In embodiments, when R^{2C} is substituted, it is substituted with at least one lower substituent group.

In embodiments, a substituted R^{2D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2D} is substituted with a plurality of groups selected from substituent groups, size-limited substituent groups, and lower substituent groups; each substituent group, size-limited substituent group, and/or lower substituent group may optionally be different. In embodiments, when R^{2D} is substituted, it is substituted with at least one substituent group. In embodiments, when R^{2D} is substituted, it is substituted with at least one size-limited substituent group. In embodiments, when R^{2D} is substituted, it is substituted with at least one lower substituent group.

In embodiments, R^{2A} is independently hydrogen. In embodiments, R^{2A} is independently substituted or unsubstituted C₁-C₅ alkyl. In embodiments, R^{2A} is independently unsubstituted methyl. In embodiments, R^{2A} is independently unsubstituted ethyl. In embodiments, R^{2A} is independently unsubstituted propyl. In embodiments, R^{2A} is independently unsubstituted n-propyl. In embodiments, R^{2A} is independently unsubstituted isopropyl. In embodiments, R^{2A} is independently unsubstituted butyl. In embodiments, R^{2A} is independently unsubstituted n-butyl. In embodiments, R^{2A} is independently unsubstituted tert-butyl.

In embodiments, R^{2B} is independently hydrogen. In embodiments, R^{2B} is independently substituted or unsubstituted C₁-C₅ alkyl. In embodiments, R^{2B} is independently unsubstituted methyl. In embodiments, R^{2B} is independently unsubstituted ethyl. In embodiments, R^{2B} is independently unsubstituted propyl. In embodiments, R^{2B} is independently unsubstituted n-propyl. In embodiments, R^{2B} is independently unsubstituted isopropyl. In embodiments, R^{2B} is independently unsubstituted butyl. In embodiments, R^{2B} is independently unsubstituted n-butyl. In embodiments, R^{2B} is independently unsubstituted tert-butyl.

In embodiments, R^{2C} is independently hydrogen. In embodiments, R^{2C} is independently substituted or unsubstituted C₁-C₅ alkyl. In embodiments, R^{2C} is independently unsubstituted methyl. In embodiments, R^{2C} is independently unsubstituted ethyl. In embodiments, R^{2C} is independently unsubstituted propyl. In embodiments, R^{2C} is independently unsubstituted n-propyl. In embodiments, R^{2C} is independently unsubstituted isopropyl. In embodiments, R^{2C} is independently unsubstituted butyl. In embodiments, R^{2C} is independently unsubstituted n-butyl. In embodiments, R^{2C} is independently unsubstituted tert-butyl.

In embodiments, R^{2D} is independently hydrogen. In embodiments, R^{2D} is independently substituted or unsubstituted C₁-C₅ alkyl. In embodiments, R^{2D} is independently unsubstituted methyl. In embodiments, R^{2D} is independently unsubstituted ethyl. In embodiments, R^{2D} is independently unsubstituted propyl. In embodiments, R^{2D} is independently unsubstituted n-propyl. In embodiments, R^{2D} is independently unsubstituted isopropyl. In embodiments, R^{2D} is independently unsubstituted butyl. In embodiments, R^{2D} is independently unsubstituted n-butyl. In embodiments, R^{2D} is independently unsubstituted tert-butyl.

In embodiments, the method is conducted under continuous flow conditions.

In embodiments, when R¹ is substituted, R¹ is substituted with one or more first substituent groups denoted by R^{1.1} as explained in the definitions section above in the description of “first substituent group(s)”. In embodiments, when an R^{1.1} substituent group is substituted, the R^{1.1} substituent group is substituted with one or more second substituent groups denoted by R^{1.2} as explained in the definitions section above in the description of “first substituent group(s)”. In embodiments, when an R^{1.2} substituent group is substituted, the R^{1.2} substituent group is substituted with one or more third substituent groups denoted by R^{1.3} as explained in the definitions section above in the description of “first substituent group(s)”. In the above embodiments, R¹, R^{1.1}, R^{1.2}, and R^{1.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R¹, R^{1.1}, R^{1.2}, and R^{1.3}, respectively.

In embodiments, when R^{1A} is substituted, R^{1A} is substituted with one or more first substituent groups denoted by R^{1A.1} as explained in the definitions section above in the description of “first substituent group(s)”. In embodiments, when an R^{1A.1} substituent group is substituted, the R^{1A.1} substituent group is substituted with one or more second substituent groups denoted by R^{1A.2} as explained in the definitions section above in the description of “first substituent group(s)”. In embodiments, when an R^{1A.2} substituent group is substituted, the R^{1A.2} substituent group is substituted with one or more third substituent groups denoted by R^{1A.3} as explained in the definitions section above in the description of “first substituent group(s)”. In the above embodiments, R^{1A}, R^{1A.1}, R^{1A.2}, and R^{1A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{1A}, R^{1A.1}, R^{1A.2}, and R^{1A.3}, respectively.

In embodiments, when R^{1B} is substituted, R^{1B} is substituted with one or more first substituent groups denoted by R^{1B.1} as explained in the definitions section above in the description of “first substituent group(s)”. In embodiments, when an R^{1B.1} substituent group is substituted, the R^{1B.1} substituent group is substituted with one or more second substituent groups denoted by R^{1B.2} as explained in the definitions section above in the description of “first substituent group(s)”. In embodiments, when an R^{1B.2} substituent group is substituted, the R^{1B.2} substituent group is substituted with one or more third substituent groups denoted by R^{1B.3} as explained in the definitions section above in the description of “first substituent group(s)”. In the above embodiments, R^{1B}, R^{1B.1}, R^{1B.2}, and R^{1B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{1B}, R^{1B.1}, R^{1B.2}, and R^{1B.3}, respectively.

In embodiments, when R^{1A} and R^{1B} substituents bonded to the same nitrogen atom are optionally joined to form a moiety that is substituted (e.g., a substituted heterocycloalkyl or substituted heteroaryl), the moiety is substituted with one or more first substituent groups denoted by R^{1A.1} as explained in the definitions section above in the description of “first substituent group(s)”. In embodiments, when an R^{1A.1} substituent group is substituted, the R^{1A.1} substituent group is substituted with one or more second substituent groups denoted by R^{1A.2} as explained in the definitions section above in the description of “first substituent group(s)”. In embodiments, when an R^{1A.2} substituent group is substituted, the R^{1A.2} substituent group is substituted with one or more third substituent groups denoted by R^{1A.3} as explained in the definitions section above in the description of “first substituent group(s)”. In the above embodiments, R^{1A.1}, R^{1A.2}, and R^{1A.3} have values corresponding to the values of R^{WW.1}, R^{WW.2} and R^{WW.3}, respectively, as explained in the definitions section above in the description of “first substituent group(s)”, wherein R^{WW.1}, R^{WW.2}, and R^{WW.3} correspond to R^{1A.1}, R^{1A.2}, and R^{1A.3}, respectively.

In embodiments, when R^{1A} and R^{1B} substituents bonded to the same nitrogen atom are optionally joined to form a moiety that is substituted (e.g., a substituted heterocycloalkyl or substituted heteroaryl), the moiety is substituted with one or more first substituent groups denoted by R^{1B.1} as explained in the definitions section above in the description of “first substituent group(s)”. In embodiments, when an R^{1B.1} substituent group is substituted, the R^{1B.1} substituent group is substituted with one or more second substituent groups denoted by R^{1B.2} as explained in the definitions section above in the description of “first substituent group(s)”. In embodiments, when an R^{1B.2} substituent group is substituted, the R^{1B.2} substituent group is substituted with one or more third substituent groups denoted by R^{1B.3} as explained in the definitions section above in the description of “first substituent group(s)”. In the above embodiments, R^{1B.1}, R^{1B.2}, and R^{1B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{1B.1}, R^{1B.2}, and R^{1B.3}, respectively.

In embodiments, when R^{1C} is substituted, R^{1C} is substituted with one or more first substituent groups denoted by R^{1C.1} as explained in the definitions section above in the description of “first substituent group(s)”. In embodiments, when an R^{1C.1} substituent group is substituted, the R^{1C.1} substituent group is substituted with one or more second substituent groups denoted by R^{1C.2} as explained in the definitions section above in the description of “first substituent group(s)”. In embodiments, when an R^{1C.2} substituent group is substituted, the R^{1C.2} substituent group is substituted with one or more third substituent groups denoted by R^{1C.3} as explained in the definitions section above in the description of “first substituent group(s)”. In the above embodiments, R^{1C}, R^{1C.1}, R^{1C.2}, and R^{1C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{1C}, R^{1C.1}, R^{1C.2}, and R^{1C.3}, respectively.

In embodiments, when R^{1D} is substituted, R^{1D} is substituted with one or more first substituent groups denoted by R^{1D.1} as explained in the definitions section above in the description of “first substituent group(s)”. In embodiments, when an R^{1D.1} substituent group is substituted, the R^{1D.1} substituent group is substituted with one or more second substituent groups denoted by R^{1D.2} as explained in the definitions section above in the description of “first substituent group(s)”. In embodiments, when an R^{1D.2} substituent group is substituted, the R^{1D.2} substituent group is substituted with one or more third substituent groups denoted by R^{1D.3} as explained in the definitions section above in the description of “first substituent group(s)”. In the above embodiments, R^{1D}, R^{1D.1}, R^{1D.2}, and R^{1D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{1D}, R^{1D.1}, R^{1D.2}, and R^{1D.3}, respectively.

In embodiments, when R² is substituted, R² is substituted with one or more first substituent groups denoted by R^{2.1} as explained in the definitions section above in the description of “first substituent group(s)”. In embodiments, when an R^{2.1} substituent group is substituted, the R^{2.1} substituent group is substituted with one or more second substituent groups denoted by R^{2.2} as explained in the definitions section above in the description of “first substituent group(s)”. In embodiments, when an R^{2.2} substituent group is substituted, the R^{2.2} substituent group is substituted with one or more third substituent groups denoted by R^{2.3} as explained in the definitions section above in the description of “first substituent group(s)”. In the above embodiments, R², R^{2.1}, R^{2.2}, and R^{2.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R², R^{2.1}, R^{2.2}, and R^{2.3}, respectively.

In embodiments, when R^{2A} is substituted, R^{2A} is substituted with one or more first substituent groups denoted by R^{2A.1} as explained in the definitions section above in the description of “first substituent group(s)”. In embodiments, when an R^{2A.1} substituent group is substituted, the R^{2A.1} substituent group is substituted with one or more second substituent groups denoted by R^{2A.2} as explained in the definitions section above in the description of “first substituent group(s)”. In embodiments, when an R^{2A.2} substituent group is substituted, the R^{2A.2} substituent group is substituted with one or more third substituent groups denoted by R^{2A.3} as explained in the definitions section above in the description of “first substituent group(s)”. In the above embodiments, R^{2A}, R^{2A.1}, R^{2A.2}, and R^{2A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{2A}, R^{2A.1}, R^{2A.2}, and R^{2A.3}, respectively.

In embodiments, when R^{2B} is substituted, R^{2B} is substituted with one or more first substituent groups denoted by R^{2B.1} as explained in the definitions section above in the description of “first substituent group(s)”. In embodiments, when an R^{2B.1} substituent group is substituted, the R^{2B.1} substituent group is substituted with one or more second substituent groups denoted by R^{2B.2} as explained in the definitions section above in the description of “first substituent group(s)”. In embodiments, when an R^{2B.2} substituent group is substituted, the R^{2B.2} substituent group is substituted with one or more third substituent groups denoted by R^{2B.3} as explained in the definitions section above in the description of “first substituent group(s)”. In the above embodiments, R^{2B}, R^{2B.1}, R^{2B.2}, and R^{2B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{2B}, R^{2B.1}, R^{2B.2}, and R^{2B.3}, respectively.

In embodiments, when R^{2A} and R^{2B} substituents bonded to the same nitrogen atom are optionally joined to form a moiety that is substituted (e.g., a substituted heterocycloalkyl or substituted heteroaryl), the moiety is substituted with one or more first substituent groups denoted by R^{2A.1} as explained in the definitions section above in the description of “first substituent group(s)”. In embodiments, when an R^{2A.1} substituent group is substituted, the R^{2A.1} substituent group is substituted with one or more second substituent groups denoted by R^{2A.2} as explained in the definitions section above in the description of “first substituent group(s)”. In embodiments, when an R^{2A.2} substituent group is substituted, the R^{2A.2} substituent group is substituted with one or more third substituent groups denoted by R^{2A.3} as explained in the definitions section above in the description of “first substituent group(s)”. In the above embodiments, R^{2A.1}, R^{2A.2}, and R^{2A.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{2A.1}, R^{2A.2}, and R^{2A.3}, respectively.

In embodiments, when R^{2A} and R^{2B} substituents bonded to the same nitrogen atom are optionally joined to form a moiety that is substituted (e.g., a substituted heterocycloalkyl or substituted heteroaryl), the moiety is substituted with one or more first substituent groups denoted by R^{2B.1} as explained in the definitions section above in the description of “first substituent group(s)”. In embodiments, when an R^{2B.1} substituent group is substituted, the R^{2B.1} substituent group is substituted with one or more second substituent groups denoted by R^{2B.2} as explained in the definitions section above in the description of “first substituent group(s)”. In embodiments, when an R^{2B.2} substituent group is substituted, the R^{2B.2} substituent group is substituted with one or more third substituent groups denoted by R^{2B.3} as explained in the definitions section above in the description of “first substituent group(s)”. In the above embodiments, R^{2B.1}, R^{2B.2}, and R^{2B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{2B.1}, R^{2B.2}, and R^{2B.3}, respectively.

In embodiments, when R^{2C} is substituted, R^{2C} is substituted with one or more first substituent groups denoted by R^{2C.1} as explained in the definitions section above in the description of “first substituent group(s)”. In embodiments, when an R^{2C.1} substituent group is substituted, the R^{2C.1} substituent group is substituted with one or more second substituent groups denoted by R^{2C.2} as explained in the definitions section above in the description of “first substituent group(s)”. In embodiments, when an R^{2C.2} substituent group is substituted, the R^{2C.2} substituent group is substituted with one or more third substituent groups denoted by R^{2C.3} explained in the definitions section above in the description of “first substituent group(s)”. In the above embodiments, R^{2C}, R^{2C.1}, R^{2C.2}, and R^{2C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{2C}, R^{2C.1}, R^{2C.2}, and R^{2C.3}, respectively.

In embodiments, when R^{2D} is substituted, R^{2D} is substituted with one or more first substituent groups denoted by R^{2D.1} as explained in the definitions section above in the description of “first substituent group(s)”. In embodiments, when an R^{2D.1} substituent group is substituted, the R^{2D.1} substituent group is substituted with one or more second substituent groups denoted by R^{2D.2} as explained in the definitions section above in the description of “first substituent group(s)”. In embodiments, when an R^{2D.2} substituent group is substituted, the R^{2D.2} substituent group is substituted with one or more third substituent groups denoted by R^{2D.3} as explained in the definitions section above in the description of “first substituent group(s)”. In the above embodiments, R^{2D}, R^{2D.1}, R^{2D.2}, and R^{2D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{2D}, R^{2D.1}, R^{2D.2}, and R^{2D.3}, respectively.

In embodiments, when R³ is substituted, R³ is substituted with one or more first substituent groups denoted by R^{3.1} as explained in the definitions section above in the description of “first substituent group(s)”. In embodiments, when an R^{3.1} substituent group is substituted, the R^{3.1} substituent group is substituted with one or more second substituent groups denoted by R^{3.2} as explained in the definitions section above in the description of “first substituent group(s)”. In embodiments, when an R^{3.2} substituent group is substituted, the R^{3.2} substituent group is substituted with one or more third substituent groups denoted by R^{3.3} as explained in the definitions section above in the description of “first substituent group(s)”. In the above embodiments, R³, R^{3.1}, R^{3.2}, and R^{3.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R³, R^{3.1}, R^{3.2}, and R^{3.3}, respectively.

In embodiments, when R⁴ is substituted, R⁴ is substituted with one or more first substituent groups denoted by R^{4.1} as explained in the definitions section above in the description of “first substituent group(s)”. In embodiments, when an R^{4.1} substituent group is substituted, the R^{4.1} substituent group is substituted with one or more second substituent groups denoted by R^{4.2} as explained in the definitions section above in the description of “first substituent group(s)”. In embodiments, when an R^{4.2} substituent group is substituted, the R^{4.2} substituent group is substituted with one or more third substituent groups denoted by R^{4.3} as explained in the definitions section above in the description of “first substituent group(s)”. In the above embodiments, R⁴, R^{4.1}, R^{4.2}, and R⁴³ have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R⁴, R^{4.1}, R^{4.2}, and R^{4.3}, respectively.

In embodiments, when R⁵ is substituted, R⁵ is substituted with one or more first substituent groups denoted by R^{5.1} as explained in the definitions section above in the description of “first substituent group(s)”. In embodiments, when an R^{5.1} substituent group is substituted, the R^{5.1} substituent group is substituted with one or more second substituent groups denoted by R^{5.2} as explained in the definitions section above in the description of “first substituent group(s)”. In embodiments, when an R^{5.2} substituent group is substituted, the R^{5.2} substituent group is substituted with one or more third substituent groups denoted by R^{5.3} as explained in the definitions section above in the description of “first substituent group(s)”. In the above embodiments, R⁵, R^{5.1}, R^{5.2}, and R^{5.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R⁵, R^{5.1}, R^{5.2} and R^{5.3}, respectively.

In embodiments, when R^{5A} is substituted, R^{5A} is substituted with one or more first substituent groups denoted by R^{5A.1} as explained in the definitions section above in the description of “first substituent group(s)”. In embodiments, when an R^{5A.1} substituent group is substituted, the R^{5A.1} substituent group is substituted with one or more second substituent groups denoted by R^{5A.2} as explained in the definitions section above in the description of “first substituent group(s)”. In embodiments, when an R^{5A.2} substituent group is substituted, the R^{5A.2} substituent group is substituted with one or more third substituent groups denoted by R^{5A.3} as explained in the definitions section above in the description of “first substituent group(s)”. In the above embodiments, R^{5A}, R^{5A.1}, R^{5A.2}, and R^{5A.3} have values corresponding to the values of R^{WW}, R^{WW.1}. R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{5A}, R^{5A.1}, R^{5A.2}, and R^{5A.3}, respectively.

In embodiments, when R^{5B} is substituted, R^{5B} is substituted with one or more first substituent groups denoted by R^{5B.1} as explained in the definitions section above in the description of “first substituent group(s)”. In embodiments, when an R^{5B.1} substituent group is substituted, the R^{5B.1} substituent group is substituted with one or more second substituent groups denoted by R^{5B.2} as explained in the definitions section above in the description of “first substituent group(s)”. In embodiments, when an R^{5B.2} substituent group is substituted, the R^{5B.2} substituent group is substituted with one or more third substituent groups denoted by R^{5B.3} as explained in the definitions section above in the description of “first substituent group(s)”. In the above embodiments, R^{5B}, R^{5B.1}, R^{5B.2}, and R^{5B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{5B}, R^{5B.1}, R^{5B.2}, and R^{5B.3}, respectively.

In embodiments, when R^{5A} and R^{5B} substituents bonded to the same nitrogen atom are optionally joined to form a moiety that is substituted (e.g., a substituted heterocycloalkyl or substituted heteroaryl), the moiety is substituted with one or more first substituent groups denoted by R^{5A.1} as explained in the definitions section above in the description of “first substituent group(s)”. In embodiments, when an R^{5A.1} substituent group is substituted, the R^{5A.1} substituent group is substituted with one or more second substituent groups denoted by R^{5A.2} as explained in the definitions section above in the description of “first substituent group(s)”. In embodiments, when an R^{5A.2} substituent group is substituted, the R^{5A.2} substituent group is substituted with one or more third substituent groups denoted by R^{5A.3} as explained in the definitions section above in the description of “first substituent group(s)”. In the above embodiments, R^{5A.1}, R^{5A.2}, and R^{5A.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{5A.1}, R^{5A.2}, and R^{5A.3}, respectively.

In embodiments, when R^{5A} and R^{5B} substituents bonded to the same nitrogen atom are optionally joined to form a moiety that is substituted (e.g., a substituted heterocycloalkyl or substituted heteroaryl), the moiety is substituted with one or more first substituent groups denoted by R^{5B.1} as explained in the definitions section above in the description of “first substituent group(s)”. In embodiments, when an R^{5B.1} substituent group is substituted, the R^{5B.1} substituent group is substituted with one or more second substituent groups denoted by R^{5B.2} as explained in the definitions section above in the description of “first substituent group(s)”. In embodiments, when an R^{5B.2} substituent group is substituted, the R^{5B.2} substituent group is substituted with one or more third substituent groups denoted by R^{5B.3} as explained in the definitions section above in the description of “first substituent group(s)”. In the above embodiments, R^{5B.1}, R^{5B.2}, and R^{5B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{5B.1}, R^{5B.2}, and R^{5B.3}, respectively.

In embodiments, when R^{5C} is substituted, R^{5C} is substituted with one or more first substituent groups denoted by R^{5C.1} as explained in the definitions section above in the description of “first substituent group(s)”. In embodiments, when an R^{5C.1} substituent group is substituted, the R^{5C.1} substituent group is substituted with one or more second substituent groups denoted by R^{5C.2} as explained in the definitions section above in the description of “first substituent group(s)”. In embodiments, when an R^{5C.2} substituent group is substituted, the R^{5C.2} substituent group is substituted with one or more third substituent groups denoted by R^{5C.3} as explained in the definitions section above in the description of “first substituent group(s)”. In the above embodiments, R^{5C}, R^{5C.1}, R^{5C.2}, and R^{5C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{5C}, R^{5C.1}, R^{5C.2}, and R^{5C.3}, respectively.

In embodiments, when R^{5D} is substituted, R^{5D} is substituted with one or more first substituent groups denoted by R^{5D.1} as explained in the definitions section above in the description of “first substituent group(s)”. In embodiments, when an R^{5D.1} substituent group is substituted, the R^{5D.1} substituent group is substituted with one or more second substituent groups denoted by R^{5D.2} as explained in the definitions section above in the description of “first substituent group(s)”. In embodiments, when an R^{5D.2} substituent group is substituted, the R^{5D.2} substituent group is substituted with one or more third substituent groups denoted by R^{5D.3} as explained in the definitions section above in the description of “first substituent group(s)”. In the above embodiments, R^{5D}, R^{5D.1}, R^{5D.2}, and R^{5D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{5D}, R^{5D.1}, R^{5D.2}, and R^{5D.3}, respectively.

In embodiments, when R⁶ is substituted, R⁶ is substituted with one or more first substituent groups denoted by R^{6.1} as explained in the definitions section above in the description of “first substituent group(s)”. In embodiments, when an R^{6.1} substituent group is substituted, the R^{6.1} substituent group is substituted with one or more second substituent groups denoted by R^{6.2} as explained in the definitions section above in the description of “first substituent group(s)”. In embodiments, when an R^{6.2} substituent group is substituted, the R^{6.2} substituent group is substituted with one or more third substituent groups denoted by R^{6.3} as explained in the definitions section above in the description of “first substituent group(s)”. In the above embodiments, R⁶, R^{6.1}, R^{6.2}, and R^{6.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R⁶, R^{6.1}, R^{6.2}, and R^{6.3}, respectively.

In embodiments, when L¹ is substituted, L¹ is substituted with one or more first substituent groups denoted by R^{L1.1} as explained in the definitions section above in the description of “first substituent group(s)”. In embodiments, when an R^{L1.1} substituent group is substituted, the R^{L1.1} substituent group is substituted with one or more second substituent groups denoted by R^{L1.2} as explained in the definitions section above in the description of “first substituent group(s)”. In embodiments, when an R^{L1.2} substituent group is substituted, the R^{L1.2} substituent group is substituted with one or more third substituent groups denoted by R^{L1.3} as explained in the definitions section above in the description of “first substituent group(s)”. In the above embodiments, L¹, R^{L1.1}, R^{L1.2}, and R^{L1.3} have values corresponding to the values of L^{WW}, R^{LWW.1}. R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L¹, R^{L1.1}, R^{L1.2}, and R^{L1.3}, respectively.

In embodiments, when L² is substituted, L² is substituted with one or more first substituent groups denoted by R^{12.1} as explained in the definitions section above in the description of “first substituent group(s)”. In embodiments, when an R^{L2.1} substituent group is substituted, the R^{12.1} substituent group is substituted with one or more second substituent groups denoted by R^{12.2} as explained in the definitions section above in the description of “first substituent group(s)”. In embodiments, when an R^{12.2} substituent group is substituted, the R^{L2.2} substituent group is substituted with one or more third substituent groups denoted by R^{12.3} as explained in the definitions section above in the description of “first substituent group(s)”. In the above embodiments, L², R^{L2.1}, R^{L2.2}, and R^{L2.3} have values corresponding to the values of L^{WW}, R^{LWW.1}. R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L², R^{L2.1}, R^{L2.2}, and R^{L2.3}, respectively.

In an aspect is provided a method of quantifying propene, the method including (a) conducting a chemical reaction that produces propene in the presence of a gaseous internal standard at a known pressure; (b) analyzing a crude reaction mixture produced by the chemical reaction by gas chromatography; (c) measuring the signal intensities.

### III. Compositions

In an aspect is provided a substituted or unsubstituted dehydrogenated polyolefin. In an aspect is provided a substituted or substituted dehydrogenated polyethylene or polypropylene. In an aspect the dehydrogenated polyethylene is produced by dehydrogenation of polyethylene. Alternatively, in some embodiments, the dehydrogenated polyethylene is not produced by dehydrogenation of polyethylene. In some embodiments, the dehydrogenated polyethylene is produced by installation of a leaving group followed by elimination. In some embodiments, the dehydrogenated polyethylene is produced by fragmentation of a precursor into fragments (e.g., cracking, pyrolysis, fluid catalytic cracking, catalytic cracking). In some embodiments, the dehydrogenation polyethylene comprises an olefin moiety. In an aspect is provided a substituted or unsubstituted dehydrogenated polyethylene, wherein a percentage of from 0.01% to 1.5% or from 1.7% to 2.3% or from 2.4% to 4.3% or from 4.5% to 13.2% of monomer units are unsaturated (e.g., olefinic, e.g., monoenes). In embodiments, a percentage of from 0.01% to 1.5% or from 1.7% to 2.3% or from 2.4% to 4.3% or from 4.5% to 13.2% of monomer units are olefinic. In embodiments, a percentage of from 0.01% to 1.5% of monomer units are olefinic. In embodiments, a percentage of from 1.7% to 2.3% of monomer units are olefinic. In embodiments, a percentage of from 1.7% to 2.3% of monomer units are olefinic. In embodiments, a percentage of from 2.4% to 4.3% of monomer units are olefinic. In embodiments, a percentage of from 4.5% to 13.2% of monomer units are olefinic.

In embodiments, the percentage of olefinic carbon units in the dehydrogenated polyethylene is at least 0.001%, at least 0.01%, at least 0.1%, at least 0.2%, at least 0.5%, at least 0.75%, at least 0.9%, at least 1%, at least 1.1%, at least 1.3%, at least 1.5%, or more. In embodiments, the percentage of olefinic carbon units in the dehydrogenated polyethylene is at most 0.001%, at most 0.01%, at most 0.1%, at most 0.2%, at most 0.5%, at most 0.75%, at most 0.9%, at most 1%, at most 1.1%, at most 1.3%, at most 1.5%, or less.

In embodiments, the percentage of olefinic carbon units in the dehydrogenated polyethylene is about 0.001% to about 1.5%. In embodiments, the percentage of olefinic carbon units in the dehydrogenated polyethylene is at least about 0.001%. In embodiments, the percentage of olefinic carbon units in the dehydrogenated polyethylene is at most about 1.5%. In embodiments, the percentage of olefinic carbon units in the dehydrogenated polyethylene is about 0.001% to about 0.01%, about 0.001% to about 0.1%, about 0.001% to about 0.2%, about 0.001% to about 0.5%, about 0.001% to about 0.75%, about 0.001% to about 0.9%, about 0.001% to about 1%, about 0.001% to about 1.1%, about 0.001% to about 1.3%, about 0.001% to about 1.4%, about 0.001% to about 1.5%, about 0.01% to about 0.1%, about 0.01% to about 0.2%, about 0.01% to about 0.5%, about 0.01% to about 0.75%, about 0.01% to about 0.9%, about 0.01% to about 1%, about 0.01% to about 1.1%, about 0.01% to about 1.3%, about 0.01% to about 1.4%, about 0.01% to about 1.5%, about 0.1% to about 0.2%, about 0.1% to about 0.5%, about 0.1% to about 0.75%, about 0.1% to about 0.9%, about 0.1% to about 1%, about 0.1% to about 1.1%, about 0.1% to about 1.3%, about 0.1% to about 1.4%, about 0.1% to about 1.5%, about 0.2% to about 0.5%, about 0.2% to about 0.75%, about 0.2% to about 0.9%, about 0.2% to about 1%, about 0.2% to about 1.1%, about 0.2% to about 1.3%, about 0.2% to about 1.4%, about 0.2% to about 1.5%, about 0.5% to about 0.75%, about 0.5% to about 0.9%, about 0.5% to about 1%, about 0.5% to about 1.1%, about 0.5% to about 1.3%, about 0.5% to about 1.4%, about 0.5% to about 1.5%, about 0.75% to about 0.9%, about 0.75% to about 1%, about 0.75% to about 1.1%, about 0.75% to about 1.3%, about 0.75% to about 1.4%, about 0.75% to about 1.5%, about 0.9% to about 1%, about 0.9% to about 1.1%, about 0.9% to about 1.3%, about 0.9% to about 1.4%, about 0.9% to about 1.5%, about 1% to about 1.1%, about 1% to about 1.3%, about 1% to about 1.4%, about 1% to about 1.5%, about 1.1% to about 1.3%, about 1.1% to about 1.4%, about 1.1% to about 1.5%, about 1.3% to about 1.4%, about 1.3% to about 1.5%, or about 1.4% to about 1.5%. In embodiments, the percentage of olefinic carbon units in the dehydrogenated polyethylene is about 0.001%, about 0.01%, about 0.1%, about 0.2%, about 0.5%, about 0.75%, about 0.9%, about 1%, about 1.1%, about 1.3%, about 1.4%, or about 1.5%.

In embodiments, the percentage of olefinic carbon units in the dehydrogenated polyethylene is at least 1.7%, at least 1.8%, at least 1.9%, at least 2.0%, at least 2.1%, at least 2.2%, at least 2.3%, or more. In embodiments, the percentage of olefinic carbon units in the dehydrogenated polyethylene is at most 1.7%, at most 1.8%, at most 1.9%, at most 2.0%, at most 2.1%, at most 2.2%, at most 2.3%, or less.

In embodiments, the percentage of olefinic carbon units in the dehydrogenated polyethylene is about 1.7% to about 2.3%. In embodiments, the percentage of olefinic carbon units in the dehydrogenated polyethylene is at least about 1.7%. In embodiments, the percentage of olefinic carbon units in the dehydrogenated polyethylene is at most about 2.3%. In embodiments, the percentage of olefinic carbon units in the dehydrogenated polyethylene is about 1.7% to about 1.8%, about 1.7% to about 1.9%, about 1.7% to about 2%, about 1.7% to about 2.1%, about 1.7% to about 2.2%, about 1.7% to about 2.3%, about 1.8% to about 1.9%, about 1.8% to about 2%, about 1.8% to about 2.1%, about 1.8% to about 2.2%, about 1.8% to about 2.3%, about 1.9% to about 2%, about 1.9% to about 2.1%, about 1.9% to about 2.2%, about 1.9% to about 2.3%, about 2% to about 2.1%, about 2% to about 2.2%, about 2% to about 2.3%, about 2.1% to about 2.2%, about 2.1% to about 2.3%, or about 2.2% to about 2.3%. In embodiments, the percentage of olefinic carbon units in the dehydrogenated polyethylene is about 1.7%, about 1.8%, about 1.9%, about 2%, about 2.1%, about 2.2%, or about 2.3%.

In embodiments, the percentage of olefinic carbon units in the dehydrogenated polyethylene is at least 2.4%, at least 2.5%, at least 2.6%, at least 2.7%, at least 2.8%, at least 2.9%, at least 3%, at least 3.3%, at least 3.5%, at least 3.7%, at least 4%, at least 4.3%, or more. In embodiments, the percentage of olefinic carbon units in the dehydrogenated polyethylene is at most 2.4%, at most 2.5%, at most 2.6%, at most 2.7%, at most 2.8%, at most 2.9%, at most 3%, at most 3.3%, at most 3.5%, at most 3.7%, at most 4%, at most 4.3%, or less. In embodiments, the percentage of olefinic carbon units in the dehydrogenated polyethylene is about 2.4% to about 4.3%. In embodiments, the percentage of olefinic carbon units in the dehydrogenated polyethylene is at least about 2.4%. In embodiments, the percentage of olefinic carbon units in the dehydrogenated polyethylene is at most about 4.3%. In embodiments, the percentage of olefinic carbon units in the dehydrogenated polyethylene is about 2.4% to about 2.5%, about 2.4% to about 2.6%, about 2.4% to about 2.7%, about 2.4% to about 2.8%, about 2.4% to about 2.9%, about 2.4% to about 3%, about 2.4% to about 3.3%, about 2.4% to about 3.5%, about 2.4% to about 3.7%, about 2.4% to about 4%, about 2.4% to about 4.3%, about 2.5% to about 2.6%, about 2.5% to about 2.7%, about 2.5% to about 2.8%, about 2.5% to about 2.9%, about 2.5% to about 3%, about 2.5% to about 3.3%, about 2.5% to about 3.5%, about 2.5% to about 3.7%, about 2.5% to about 4%, about 2.5% to about 4.3%, about 2.6% to about 2.7%, about 2.6% to about 2.8%, about 2.6% to about 2.9%, about 2.6% to about 3%, about 2.6% to about 3.3%, about 2.6% to about 3.5%, about 2.6% to about 3.7%, about 2.6% to about 4%, about 2.6% to about 4.3%, about 2.7% to about 2.8%, about 2.7% to about 2.9%, about 2.7% to about 3%, about 2.7% to about 3.3%, about 2.7% to about 3.5%, about 2.7% to about 3.7%, about 2.7% to about 4%, about 2.7% to about 4.3%, about 2.8% to about 2.9%, about 2.8% to about 3%, about 2.8% to about 3.3%, about 2.8% to about 3.5%, about 2.8% to about 3.7%, about 2.8% to about 4%, about 2.8% to about 4.3%, about 2.9% to about 3%, about 2.9% to about 3.3%, about 2.9% to about 3.5%, about 2.9% to about 3.7%, about 2.9% to about 4%, about 2.9% to about 4.3%, about 3% to about 3.3%, about 3% to about 3.5%, about 3% to about 3.7%, about 3% to about 4%, about 3% to about 4.3%, about 3.3% to about 3.5%, about 3.3% to about 3.7%, about 3.3% to about 4%, about 3.3% to about 4.3%, about 3.5% to about 3.7%, about 3.5% to about 4%, about 3.5% to about 4.3%, about 3.7% to about 4%, about 3.7% to about 4.3%, or about 4% to about 4.3%. In embodiments, the percentage of olefinic carbon units in the dehydrogenated polyethylene is about 2.4%, about 2.5%, about 2.6%, about 2.7%, about 2.8%, about 2.9%, about 3%, about 3.3%, about 3.5%, about 3.7%, about 4%, or about 4.3%.

In embodiments, the percentage of olefinic carbon units in the dehydrogenated polyethylene is at least 4.5%, at least 4.7%, at least 5%, at least 5.2%, at least 5.5%, at least 6%, at least 7%, at least 8%, at least 9%, at least 10%, at least 11%, at least 12%, at least 13%, at least 13.3%, or more. In embodiments, the percentage of olefinic carbon units in the dehydrogenated polyethylene is at most 4.5%, at most 4.7%, at most 5%, at most 5.2%, at most 5.5%, at most 6%, at most 7%, at most 8%, at most 9%, at most 10%, at most 11%, at most 12%, at most 13%, at most 13.3%, or more.

In embodiments, the percentage of olefinic carbon units in the dehydrogenated polyethylene is about 4.5% to about 13.3%. In embodiments, the percentage of olefinic carbon units in the dehydrogenated polyethylene is at least about 4.5%. In embodiments, the percentage of olefinic carbon units in the dehydrogenated polyethylene is at most about 13.3%. In embodiments, the percentage of olefinic carbon units in the dehydrogenated polyethylene is about 4.5% to about 4.7%, about 4.5% to about 5%, about 4.5% to about 5.2%, about 4.5% to about 5.5%, about 4.5% to about 6%, about 4.5% to about 7%, about 4.5% to about 8%, about 4.5% to about 9%, about 4.5% to about 10%, about 4.5% to about 12%, about 4.5% to about 13.3%, about 4.7% to about 5%, about 4.7% to about 5.2%, about 4.7% to about 5.5%, about 4.7% to about 6%, about 4.7% to about 7%, about 4.7% to about 8%, about 4.7% to about 9%, about 4.7% to about 10%, about 4.7% to about 12%, about 4.7% to about 13.3%, about 5% to about 5.2%, about 5% to about 5.5%, about 5% to about 6%, about 5% to about 7%, about 5% to about 8%, about 5% to about 9%, about 5% to about 10%, about 5% to about 12%, about 5% to about 13.3%, about 5.2% to about 5.5%, about 5.2% to about 6%, about 5.2% to about 7%, about 5.2% to about 8%, about 5.2% to about 9%, about 5.2% to about 10%, about 5.2% to about 12%, about 5.2% to about 13.3%, about 5.5% to about 6%, about 5.5% to about 7%, about 5.5% to about 8%, about 5.5% to about 9%, about 5.5% to about 10%, about 5.5% to about 12%, about 5.5% to about 13.3%, about 6% to about 7%, about 6% to about 8%, about 6% to about 9%, about 6% to about 10%, about 6% to about 12%, about 6% to about 13.3%, about 7% to about 8%, about 7% to about 9%, about 7% to about 10%, about 7% to about 12%, about 7% to about 13.3%, about 8% to about 9%, about 8% to about 10%, about 8% to about 12%, about 8% to about 13.3%, about 9% to about 10%, about 9% to about 12%, about 9% to about 13.3%, about 10% to about 12%, about 10% to about 13.3%, or about 12% to about 13.3%. In embodiments, the percentage of olefinic carbon units in the dehydrogenated polyethylene is about 4.5%, about 4.7%, about 5%, about 5.2%, about 5.5%, about 6%, about 7%, about 8%, about 9%, about 10%, about 12%, or about 13.3%.

In an aspect is provided a substituted or unsubstituted dehydrogenated polyethylene, wherein a percentage of from 0.01% to 1.5% or from 1.7% to 2.3% or from 2.4% to 4.3% or from 4.5% to 13.2% of repeating ethylene units of a polymer are olefinic.

In some embodiments, the percentage of olefinic carbons in the dehydrogenated polyethylene is at least 0.62%, at least 0.81%, at least 1.06%, at least 1.09%, at least 1.34%, at least 1.42%, at least 1.46%, at least 1.76%, at least 2.17%, at least 2.3%, at least 2.38%, at least 2.55%, at least 3.54%, or more. In some embodiments, the percentage of olefinic carbons in the dehydrogenated polyethylene is at most 0.62%, at most 0.81%, at most 1.06%, at most 1.09%, at most 1.34%, at most 1.42%, at most 1.46%, at most 1.76%, at most 2.17%, at most 2.3%, at most 2.38%, at most 2.55%, at most 3.54%, or less.

In some embodiments, the percentage of olefinic carbons in the dehydrogenated polyethylene is about 0.1% to about 2.38%. In some embodiments, the percentage of olefinic carbons in the dehydrogenated polyethylene is at least about 0.1%. In some embodiments, the percentage of olefinic carbons in the dehydrogenated polyethylene is at most about 2.38%. In some embodiments, the percentage of olefinic carbons in the dehydrogenated polyethylene is about 0.1% to about 0.62%, about 0.1% to about 0.81%, about 0.1% to about 1.06%, about 0.1% to about 1.09%, about 0.1% to about 1.34%, about 0.1% to about 1.42%, about 0.1% to about 1.46%, about 0.1% to about 1.76%, about 0.1% to about 2.17%, about 0.1% to about 2.3%, about 0.1% to about 2.38%, about 0.62% to about 0.81%, about 0.62% to about 1.06%, about 0.62% to about 1.09%, about 0.62% to about 1.34%, about 0.62% to about 1.42%, about 0.62% to about 1.46%, about 0.62% to about 1.76%, about 0.62% to about 2.17%, about 0.62% to about 2.3%, about 0.62% to about 2.38%, about 0.81% to about 1.06%, about 0.81% to about 1.09%, about 0.81% to about 1.34%, about 0.81% to about 1.42%, about 0.81% to about 1.46%, about 0.81% to about 1.76%, about 0.81% to about 2.17%, about 0.81% to about 2.3%, about 0.81% to about 2.38%, about 1.06% to about 1.09%, about 1.06% to about 1.34%, about 1.06% to about 1.42%, about 1.06% to about 1.46%, about 1.06% to about 1.76%, about 1.06% to about 2.17%, about 1.06% to about 2.3%, about 1.06% to about 2.38%, about 1.09% to about 1.34%, about 1.09% to about 1.42%, about 1.09% to about 1.46%, about 1.09% to about 1.76%, about 1.09% to about 2.17%, about 1.09% to about 2.3%, about 1.09% to about 2.38%, about 1.34% to about 1.42%, about 1.34% to about 1.46%, about 1.34% to about 1.76%, about 1.34% to about 2.17%, about 1.34% to about 2.3%, about 1.34% to about 2.38%, about 1.42% to about 1.46%, about 1.42% to about 1.76%, about 1.42% to about 2.17%, about 1.42% to about 2.3%, about 1.42% to about 2.38%, about 1.46% to about 1.76%, about 1.46% to about 2.17%, about 1.46% to about 2.3%, about 1.46% to about 2.38%, about 1.76% to about 2.17%, about 1.76% to about 2.3%, about 1.76% to about 2.38%, about 2.17% to about 2.3%, about 2.17% to about 2.38%, or about 2.3% to about 2.38%. In some embodiments, the percentage of olefinic carbons in the dehydrogenated polyethylene is about 0.1%, about 0.62%, about 0.81%, about 1.06%, about 1.09%, about 1.34%, about 1.42%, about 1.46%, about 1.76%, about 2.17%, about 2.3%, or about 2.38%.

In some embodiments, the percentage of olefinic carbons in the dehydrogenated polyethylene is about 0.1% to about 10%. In some embodiments, the percentage of olefinic carbons in the dehydrogenated polyethylene is at least about 0.1%. In some embodiments, the percentage of olefinic carbons in the dehydrogenated polyethylene is at most about 10%. In some embodiments, the percentage of olefinic carbons in the dehydrogenated polyethylene is about 0.1% to about 0.62%, about 0.1% to about 0.81%, about 0.1% to about 1.34%, about 0.1% to about 1.09%, about 0.1% to about 1.34%, about 0.1% to about 1.76%, about 0.1% to about 2.38%, about 0.1% to about 2.55%, about 0.1% to about 3.54%, about 0.1% to about 4%, about 0.1% to about 10%, about 0.62% to about 0.81%, about 0.62% to about 1.34%, about 0.62% to about 1.09%, about 0.62% to about 1.34%, about 0.62% to about 1.76%, about 0.62% to about 2.38%, about 0.62% to about 2.55%, about 0.62% to about 3.54%, about 0.62% to about 4%, about 0.62% to about 10%, about 0.81% to about 1.34%, about 0.81% to about 1.09%, about 0.81% to about 1.34%, about 0.81% to about 1.76%, about 0.81% to about 2.38%, about 0.81% to about 2.55%, about 0.81% to about 3.54%, about 0.81% to about 4%, about 0.81% to about 10%, about 1.34% to about 1.09%, about 1.34% to about 1.34%, about 1.34% to about 1.76%, about 1.34% to about 2.38%, about 1.34% to about 2.55%, about 1.34% to about 3.54%, about 1.34% to about 4%, about 1.34% to about 10%, about 1.09% to about 1.34%, about 1.09% to about 1.76%, about 1.09% to about 2.38%, about 1.09% to about 2.55%, about 1.09% to about 3.54%, about 1.09% to about 4%, about 1.09% to about 10%, about 1.34% to about 1.76%, about 1.34% to about 2.38%, about 1.34% to about 2.55%, about 1.34% to about 3.54%, about 1.34% to about 4%, about 1.34% to about 10%, about 1.76% to about 2.38%, about 1.76% to about 2.55%, about 1.76% to about 3.54%, about 1.76% to about 4%, about 1.76% to about 10%, about 2.38% to about 2.55%, about 2.38% to about 3.54%, about 2.38% to about 4%, about 2.38% to about 10%, about 2.55% to about 3.54%, about 2.55% to about 4%, about 2.55% to about 10%, about 3.54% to about 4%, about 3.54% to about 10%, or about 4% to about 10%. In some embodiments, the percentage of olefinic carbons in the dehydrogenated polyethylene is about 0.1%, about 0.62%, about 0.81%, about 1.34%, about 1.09%, about 1.34%, about 1.76%, about 2.38%, about 2.55%, about 3.54%, about 4%, or about 10%.

In some embodiments, the percentage of olefinic carbons in the dehydrogenated polyethylene is about 0.1% to about 20%. In some embodiments, the percentage of olefinic carbons in the dehydrogenated polyethylene is at least about 0.1%. In some embodiments, the percentage of olefinic carbons in the dehydrogenated polyethylene is at most about 20%. In some embodiments, the percentage of olefinic carbons in the dehydrogenated polyethylene is about 0.1% to about 1%, about 0.1% to about 1.5%, about 0.1% to about 1.75%, about 0.1% to about 2%, about 0.1% to about 2.5%, about 0.1% to about 3%, about 0.1% to about 4%, about 0.1% to about 5%, about 0.1% to about 7.5%, about 0.1% to about 10%, about 0.1% to about 20%, about 1% to about 1.5%, about 1% to about 1.75%, about 1% to about 2%, about 1% to about 2.5%, about 1% to about 3%, about 1% to about 4%, about 1% to about 5%, about 1% to about 7.5%, about 1% to about 10%, about 1% to about 20%, about 1.5% to about 1.75%, about 1.5% to about 2%, about 1.5% to about 2.5%, about 1.5% to about 3%, about 1.5% to about 4%, about 1.5% to about 5%, about 1.5% to about 7.5%, about 1.5% to about 10%, about 1.5% to about 20%, about 1.75% to about 2%, about 1.75% to about 2.5%, about 1.75% to about 3%, about 1.75% to about 4%, about 1.75% to about 5%, about 1.75% to about 7.5%, about 1.75% to about 10%, about 1.75% to about 20%, about 2% to about 2.5%, about 2% to about 3%, about 2% to about 4%, about 2% to about 5%, about 2% to about 7.5%, about 2% to about 10%, about 2% to about 20%, about 2.5% to about 3%, about 2.5% to about 4%, about 2.5% to about 5%, about 2.5% to about 7.5%, about 2.5% to about 10%, about 2.5% to about 20%, about 3% to about 4%, about 3% to about 5%, about 3% to about 7.5%, about 3% to about 10%, about 3% to about 20%, about 4% to about 5%, about 4% to about 7.5%, about 4% to about 10%, about 4% to about 20%, about 5% to about 7.5%, about 5% to about 10%, about 5% to about 20%, about 7.5% to about 10%, about 7.5% to about 20%, or about 10% to about 20%.

In some embodiments, the percentage of olefinic carbons in the dehydrogenated polyethylene is about 0.1%, about 1%, about 1.5%, about 1.75%, about 2%, about 2.5%, about 3%, about 4%, about 5%, about 7.5%, about 10%, or about 20%.

In embodiments, the percentage of olefinic carbons in the dehydrogenated polyethylene is at least about 0.01%, at least about 0.05%, at least about 0.1%, at least about 0.15%, at least about 0.17%, at least about 0.19%, at least about 0.2%, at least about 0.22%, at least about 0.25%, at least about 0.3%, at least about 0.35%, at least about 0.38%, at least about 0.4%, at least about 0.45%, at least about 0.5%, at least about 0.52%, at least about 0.55%, at least about 0.56, at least about 0.6%, at least about 0.65%, at least about 0.75%, at least about 1%, at least about 2%, at least about 3%, at least about 4%, at least about 5%, at least about 10%, or more.

In embodiments, the percentage of olefinic carbons in the dehydrogenated polyethylene is at most about 0.01%, at most about 0.05%, at most about 0.1%, at most about 0.15%, at most about 0.17%, at most about 0.19%, at most about 0.2%, at most about 0.22%, at most about 0.25%, at most about 0.3%, at most about 0.35%, at most about 0.38%, at most about 0.4%, at most about 0.45%, at most about 0.5%, at most about 0.52%, at most about 0.55%, at most about 0.56, at most about 0.6%, at most about 0.65%, at most about 0.75%, at most about 1%, at most about 2%, at most about 3%, at most about 4%, at most about 5%, at most about 10%, or less.

In some embodiments, the percentage of olefinic carbons in the dehydrogenated polyethylene is about 0.01% to about 10%. In some embodiments, the percentage of olefinic carbons in the dehydrogenated polyethylene is at least about 0.01%. In some embodiments, the percentage of olefinic carbons in the dehydrogenated polyethylene is at most about 10%. In some embodiments, the percentage of olefinic carbons in the dehydrogenated polyethylene is about 0.01% to about 0.05%, about 0.01% to about 0.1%, about 0.01% to about 0.19%, about 0.01% to about 0.22%, about 0.01% to about 0.25%, about 0.01% to about 0.38%, about 0.01% to about 0.52%, about 0.01% to about 0.56%, about 0.01% to about 1%, about 0.01% to about 2%, about 0.01% to about 10%, about 0.05% to about 0.1%, about 0.05% to about 0.19%, about 0.05% to about 0.22%, about 0.05% to about 0.25%, about 0.05% to about 0.38%, about 0.05% to about 0.52%, about 0.05% to about 0.56%, about 0.05% to about 1%, about 0.05% to about 2%, about 0.05% to about 10%, about 0.1% to about 0.19%, about 0.1% to about 0.22%, about 0.1% to about 0.25%, about 0.1% to about 0.38%, about 0.1% to about 0.52%, about 0.1% to about 0.56%, about 0.1% to about 1%, about 0.1% to about 2%, about 0.1% to about 10%, about 0.19% to about 0.22%, about 0.19% to about 0.25%, about 0.19% to about 0.38%, about 0.19% to about 0.52%, about 0.19% to about 0.56%, about 0.19% to about 1%, about 0.19% to about 2%, about 0.19% to about 10%, about 0.22% to about 0.25%, about 0.22% to about 0.38%, about 0.22% to about 0.52%, about 0.22% to about 0.56%, about 0.22% to about 1%, about 0.22% to about 2%, about 0.22% to about 10%, about 0.25% to about 0.38%, about 0.25% to about 0.52%, about 0.25% to about 0.56%, about 0.25% to about 1%, about 0.25% to about 2%, about 0.25% to about 10%, about 0.38% to about 0.52%, about 0.38% to about 0.56%, about 0.38% to about 1%, about 0.38% to about 2%, about 0.38% to about 10%, about 0.52% to about 0.56%, about 0.52% to about 1%, about 0.52% to about 2%, about 0.52% to about 10%, about 0.56% to about 1%, about 0.56% to about 2%, about 0.56% to about 10%, about 1% to about 2%, about 1% to about 10%, or about 2% to about 10%. In some embodiments, the percentage of olefinic carbons in the dehydrogenated polyethylene is about 0.01%, about 0.05%, about 0.1%, about 0.19%, about 0.22%, about 0.25%, about 0.38%, about 0.52%, about 0.56%, about 1%, about 2%, or about 10%.

In an aspect is provided a substituted or unsubstituted dehydrogenated polyethylene, having a number average molecular weight of 2,200 Da or greater.

In embodiments, the substituted or unsubstituted dehydrogenated polyethylene has a number average molecular weight of from about 1,000 Da to about 500,000 Da. In embodiments, the substituted or unsubstituted dehydrogenated polyethylene has a number average molecular weight of from about 1,000 Da to about 400,000 Da. In embodiments, the substituted or unsubstituted dehydrogenated polyethylene has a number average molecular weight of from about 1,000 Da to about 300,000 Da. In embodiments, the substituted or unsubstituted dehydrogenated polyethylene has a number average molecular weight of from about 1,000 Da to about 200,000 Da. In embodiments, the substituted or unsubstituted dehydrogenated polyethylene has a number average molecular weight of from about 1,000 Da to about 100,000 Da. In embodiments, the substituted or unsubstituted dehydrogenated polyethylene has a number average molecular weight of from about 1,000 Da to about 50,000 Da. In embodiments, the substituted or unsubstituted dehydrogenated polyethylene has a number average molecular weight of from about 1,000 Da to about 40,000 Da. In embodiments, the substituted or unsubstituted dehydrogenated polyethylene has a number average molecular weight of from about 1,000 Da to about 30,000 Da. In embodiments, the substituted or unsubstituted dehydrogenated polyethylene has a number average molecular weight of from about 1,000 Da to about 20,000 Da. In embodiments, the substituted or unsubstituted dehydrogenated polyethylene has a number average molecular weight of from about 1,000 Da to about 10,000 Da. In embodiments, the substituted or unsubstituted dehydrogenated polyethylene has a number average molecular weight of from about 2,200 Da to about 500,000 Da. In embodiments, the substituted or unsubstituted dehydrogenated polyethylene has a number average molecular weight of from about 2,200 Da to about 400,000 Da. In embodiments, the substituted or unsubstituted dehydrogenated polyethylene has a number average molecular weight of from about 2,200 Da to about 300,000 Da. In embodiments, the substituted or unsubstituted dehydrogenated polyethylene has a number average molecular weight of from about 2,200 Da to about 200,000 Da. In embodiments, the substituted or unsubstituted dehydrogenated polyethylene has a number average molecular weight of from about 2,200 Da to about 100,000 Da. In embodiments, the substituted or unsubstituted dehydrogenated polyethylene has a number average molecular weight of from about 2,200 Da to about 50,000 Da. In embodiments, the substituted or unsubstituted dehydrogenated polyethylene has a number average molecular weight of from about 2,200 Da to about 40,000 Da. In embodiments, the substituted or unsubstituted dehydrogenated polyethylene has a number average molecular weight of from about 2,200 Da to about 30,000 Da. In embodiments, the substituted or unsubstituted dehydrogenated polyethylene has a number average molecular weight of from about 2,200 Da to about 20,000 Da. In embodiments, the substituted or unsubstituted dehydrogenated polyethylene has a number average molecular weight of from about 2,200 Da to about 10,000 Da. In embodiments, the substituted or unsubstituted dehydrogenated polyethylene has a number average molecular weight of from about 2,200 Da to about 6,500 Da or a number average molecular weight greater than about 6,700 Da.

In embodiments, the substituted or unsubstituted dehydrogenated polyethylene has a number average molecular weight of from 1,000 Da to 500,000 Da. In embodiments, the substituted or unsubstituted dehydrogenated polyethylene has a number average molecular weight of from 1,000 Da to 400,000 Da. In embodiments, the substituted or unsubstituted dehydrogenated polyethylene has a number average molecular weight of from 1,000 Da to 300,000 Da. In embodiments, the substituted or unsubstituted dehydrogenated polyethylene has a number average molecular weight of from 1,000 Da to 200,000 Da. In embodiments, the substituted or unsubstituted dehydrogenated polyethylene has a number average molecular weight of from 1,000 Da to 100,000 Da. In embodiments, the substituted or unsubstituted dehydrogenated polyethylene has a number average molecular weight of from 1,000 Da to 50,000 Da. In embodiments, the substituted or unsubstituted dehydrogenated polyethylene has a number average molecular weight of from 1,000 Da to 40,000 Da. In embodiments, the substituted or unsubstituted dehydrogenated polyethylene has a number average molecular weight of from 1,000 Da to 30,000 Da. In embodiments, the substituted or unsubstituted dehydrogenated polyethylene has a number average molecular weight of from 1,000 Da to 20,000 Da. In embodiments, the substituted or unsubstituted dehydrogenated polyethylene has a number average molecular weight of from 1,000 Da to 10,000 Da. In embodiments, the substituted or unsubstituted dehydrogenated polyethylene has a number average molecular weight of from 2,200 Da to 500,000 Da. In embodiments, the substituted or unsubstituted dehydrogenated polyethylene has a number average molecular weight of from 2,200 Da to 400,000 Da. In embodiments, the substituted or unsubstituted dehydrogenated polyethylene has a number average molecular weight of from 2,200 Da to 300,000 Da. In embodiments, the substituted or unsubstituted dehydrogenated polyethylene has a number average molecular weight of from 2,200 Da to 200,000 Da. In embodiments, the substituted or unsubstituted dehydrogenated polyethylene has a number average molecular weight of from 2,200 Da to 100,000 Da. In embodiments, the substituted or unsubstituted dehydrogenated polyethylene has a number average molecular weight of from 2,200 Da to 50,000 Da. In embodiments, the substituted or unsubstituted dehydrogenated polyethylene has a number average molecular weight of from 2,200 Da to 40,000 Da. In embodiments, the substituted or unsubstituted dehydrogenated polyethylene has a number average molecular weight of from 2,200 Da to 30,000 Da. In embodiments, the substituted or unsubstituted dehydrogenated polyethylene has a number average molecular weight of from 2,200 Da to 20,000 Da. In embodiments, the substituted or unsubstituted dehydrogenated polyethylene has a number average molecular weight of from 2,200 Da to 10,000 Da. In embodiments, the substituted or unsubstituted dehydrogenated polyethylene has a number average molecular weight of from 2,200 Da to 6,500 Da or a number average molecular weight greater than 6,700 Da.

In embodiments, the number average molecular weight of the substituted or unsubstituted dehydrogenated polyethylene is at least about 0.5 kDa, at least about 1 kDa, at least about 2 kDa, at least about 4 kDa, at least about 5 kDa, at least about 5.22 kDa, at least about 6 kDa, at least about 7 kDa, at least about 8 kDa, at least about 9 kDa, at least about 10 kDa, at least about 11 kDa, at least about 12 kDa, at least about 13 kDa, at least about 14 kDa, at least about 15 kDa, at least about 16 kDa, at least about 17 kDa, at least about 18 kDa, at least about 18.2 kDa, at least about 19 kDa, at least about 20 kDa, at least about 21 kDa, at least about 25 kDa, at least about 30 kDa, at least about 35 kDa, at least about 40 kDa, at least about 45 kDa, or at least about 50 kDa, or more.

Alternatively, or in addition, in embodiments, the number average molecular weight of the substituted or unsubstituted dehydrogenated polyethylene is at most about 0.5 kDa, at most about 1 kDa, at most about 2 kDa, at most about 4 kDa, at most about 5 kDa, at most about 5.22 kDa, at most about 6 kDa, at most about 7 kDa, at most about 8 kDa, at most about 9 kDa, at most about 10 kDa, at most about 11 kDa, at most about 12 kDa, at most about 13 kDa, at most about 14 kDa, at most about 15 kDa, at most about 16 kDa, at most about 17 kDa, at most about 18 kDa, at most about 18.2 kDa, at most about 19 kDa, at most about 20 kDa, at most about 21 kDa, at most about 25 kDa, at most about 30 kDa, at most about 35 kDa, at most about 40 kDa, at most about 45 kDa, at most about 50 kDa, or less.

In embodiments, the number average molecular weight of the substituted or unsubstituted dehydrogenated polyethylene is at most about 5.22 kDa, or at least about 18.2 kDa, more more. In embodiments, the number average molecular weight of the substituted or unsubstituted dehydrogenated polyethylene is at least about 5.22 kDa, or at least about 18.2 kDa, or less.

In embodiments, the number average molecular weight of the substituted or unsubstituted dehydrogenated polyethylene is about 2 kDa to about 50 kDa. In embodiments, the number average molecular weight of the substituted or unsubstituted dehydrogenated polyethylene is at least about 2 kDa. In embodiments, the number average molecular weight of the substituted or unsubstituted dehydrogenated polyethylene is at most about 50 kDa. In embodiments, the number average molecular weight of the substituted or unsubstituted dehydrogenated polyethylene is about 2 kDa to about 4 kDa, about 2 kDa to about 5 kDa, about 2 kDa to about 5.22 kDa, about 2 kDa to about 8 kDa, about 2 kDa to about 10 kDa, about 2 kDa to about 12 kDa, about 2 kDa to about 15 kDa, about 2 kDa to about 18.2 kDa, about 2 kDa to about 20 kDa, about 2 kDa to about 35 kDa, about 2 kDa to about 50 kDa, about 4 kDa to about 5 kDa, about 4 kDa to about 5.22 kDa, about 4 kDa to about 8 kDa, about 4 kDa to about 10 kDa, about 4 kDa to about 12 kDa, about 4 kDa to about 15 kDa, about 4 kDa to about 18.2 kDa, about 4 kDa to about 20 kDa, about 4 kDa to about 35 kDa, about 4 kDa to about 50 kDa, about 5 kDa to about 5.22 kDa, about 5 kDa to about 8 kDa, about 5 kDa to about 10 kDa, about 5 kDa to about 12 kDa, about 5 kDa to about 15 kDa, about 5 kDa to about 18.2 kDa, about 5 kDa to about 20 kDa, about 5 kDa to about 35 kDa, about 5 kDa to about 50 kDa, about 5.22 kDa to about 8 kDa, about 5.22 kDa to about 10 kDa, about 5.22 kDa to about 12 kDa, about 5.22 kDa to about 15 kDa, about 5.22 kDa to about 18.2 kDa, about 5.22 kDa to about 20 kDa, about 5.22 kDa to about 35 kDa, about 5.22 kDa to about 50 kDa, about 8 kDa to about 10 kDa, about 8 kDa to about 12 kDa, about 8 kDa to about 15 kDa, about 8 kDa to about 18.2 kDa, about 8 kDa to about 20 kDa, about 8 kDa to about 35 kDa, about 8 kDa to about 50 kDa, about 10 kDa to about 12 kDa, about 10 kDa to about 15 kDa, about 10 kDa to about 18.2 kDa, about 10 kDa to about 20 kDa, about 10 kDa to about 35 kDa, about 10 kDa to about 50 kDa, about 12 kDa to about 15 kDa, about 12 kDa to about 18.2 kDa, about 12 kDa to about 20 kDa, about 12 kDa to about 35 kDa, about 12 kDa to about 50 kDa, about 15 kDa to about 18.2 kDa, about 15 kDa to about 20 kDa, about 15 kDa to about 35 kDa, about 15 kDa to about 50 kDa, about 18.2 kDa to about 20 kDa, about 18.2 kDa to about 35 kDa, about 18.2 kDa to about 50 kDa, about 20 kDa to about 35 kDa, about 20 kDa to about 50 kDa, or about 35 kDa to about 50 kDa. In embodiments, the number average molecular weight of the substituted or unsubstituted dehydrogenated polyethylene is about 2 kDa, about 4 kDa, about 5 kDa, about 5.22 kDa, about 8 kDa, about 10 kDa, about 12 kDa, about 15 kDa, about 18.2 kDa, about 20 kDa, about 35 kDa, or about 50 kDa.

In embodiments, the number average molecular weight of the substituted or unsubstituted dehydrogenated polyethylene is at least about 0.5 kDa, at least about 1 kDa, at least about 1.23 kDa, at least about 1.67 kDa, at least about 2 kDa, at least about 2.24 kDa, at least about 3 kDa, at least about 3.9 kDa, at least about 4 kDa, at least about 4.07 kDa, at least about 5 kDa, at least about 5.22 kDa, at least about 6 kDa, at least about 7 kDa, at least about 8 kDa, at least about 9 kDa, at least about 10 kDa, at least about 11 kDa, at least about 12 kDa, at least about 13 kDa, at least about 14 kDa, at least about 15 kDa, at least about 16 kDa, at least about 17 kDa, at least about 18 kDa, at least about 18.2 kDa, at least about 19 kDa, at least about 20 kDa, at least about 21 kDa, at least about 25 kDa, at least about 30 kDa, at least about 35 kDa, at least about 40 kDa, at least about 45 kDa, or at least about 50 kDa, or at least about 100 kDa, or at least about 200 kDa, or more.

Alternatively, or in addition, in embodiments, the number average molecular weight of the substituted or unsubstituted dehydrogenated polyethylene is at most about 0.5 kDa, at most about 1 kDa, at most about 1.23 kDa, at most about 1.67 kDa, at most about 2 kDa, at most about 2.24 kDa, at most about 3 kDa, at most about 3.9 kDa, at most about 4 kDa, at most about 4.07 kDa, at most about 5 kDa, at most about 5.22 kDa, at most about 6 kDa, at most about 7 kDa, at most about 8 kDa, at most about 9 kDa, at most about 10 kDa, at most about 11 kDa, at most about 12 kDa, at most about 13 kDa, at most about 14 kDa, at most about 15 kDa, at most about 16 kDa, at most about 17 kDa, at most about 18 kDa, at most about 18.2 kDa, at most about 19 kDa, at most about 20 kDa, at most about 21 kDa, at most about 25 kDa, at most about 30 kDa, at most about 35 kDa, at most about 40 kDa, at most about 45 kDa, or at most about 50 kDa, or at most about 100 kDa, or at most about 200 kDa, or less.

In embodiments, the number average molecular weight of the substituted or unsubstituted dehydrogenated polyethylene is about 0.5 kDa to about 50 kDa. In embodiments, the number average molecular weight of the substituted or unsubstituted dehydrogenated polyethylene is at least about 0.5 kDa. In embodiments, the number average molecular weight of the substituted or unsubstituted dehydrogenated polyethylene is at most about 50 kDa. In embodiments, the number average molecular weight of the substituted or unsubstituted dehydrogenated polyethylene is about 0.5 kDa to about 1 kDa, about 0.5 kDa to about 1.23 kDa, about 0.5 kDa to about 1.67 kDa, about 0.5 kDa to about 2 kDa, about 0.5 kDa to about 2.24 kDa, about 0.5 kDa to about 3 kDa, about 0.5 kDa to about 3.9 kDa, about 0.5 kDa to about 4.07 kDa, about 0.5 kDa to about 5.22 kDa, about 0.5 kDa to about 10 kDa, about 0.5 kDa to about 50 kDa, about 1 kDa to about 1.23 kDa, about 1 kDa to about 1.67 kDa, about 1 kDa to about 2 kDa, about 1 kDa to about 2.24 kDa, about 1 kDa to about 3 kDa, about 1 kDa to about 3.9 kDa, about 1 kDa to about 4.07 kDa, about 1 kDa to about 5.22 kDa, about 1 kDa to about 10 kDa, about 1 kDa to about 50 kDa, about 1.23 kDa to about 1.67 kDa, about 1.23 kDa to about 2 kDa, about 1.23 kDa to about 2.24 kDa, about 1.23 kDa to about 3 kDa, about 1.23 kDa to about 3.9 kDa, about 1.23 kDa to about 4.07 kDa, about 1.23 kDa to about 5.22 kDa, about 1.23 kDa to about 10 kDa, about 1.23 kDa to about 50 kDa, about 1.67 kDa to about 2 kDa, about 1.67 kDa to about 2.24 kDa, about 1.67 kDa to about 3 kDa, about 1.67 kDa to about 3.9 kDa, about 1.67 kDa to about 4.07 kDa, about 1.67 kDa to about 5.22 kDa, about 1.67 kDa to about 10 kDa, about 1.67 kDa to about 50 kDa, about 2 kDa to about 2.24 kDa, about 2 kDa to about 3 kDa, about 2 kDa to about 3.9 kDa, about 2 kDa to about 4.07 kDa, about 2 kDa to about 5.22 kDa, about 2 kDa to about 10 kDa, about 2 kDa to about 50 kDa, about 2.24 kDa to about 3 kDa, about 2.24 kDa to about 3.9 kDa, about 2.24 kDa to about 4.07 kDa, about 2.24 kDa to about 5.22 kDa, about 2.24 kDa to about 10 kDa, about 2.24 kDa to about 50 kDa, about 3 kDa to about 3.9 kDa, about 3 kDa to about 4.07 kDa, about 3 kDa to about 5.22 kDa, about 3 kDa to about 10 kDa, about 3 kDa to about 50 kDa, about 3.9 kDa to about 4.07 kDa, about 3.9 kDa to about 5.22 kDa, about 3.9 kDa to about 10 kDa, about 3.9 kDa to about 50 kDa, about 4.07 kDa to about 5.22 kDa, about 4.07 kDa to about 10 kDa, about 4.07 kDa to about 50 kDa, about 5.22 kDa to about 10 kDa, about 5.22 kDa to about 50 kDa, or about 10 kDa to about 50 kDa. In embodiments, the number average molecular weight of the substituted or unsubstituted dehydrogenated polyethylene is about 0.5 kDa, about 1 kDa, about 1.23 kDa, about 1.67 kDa, about 2 kDa, about 2.24 kDa, about 3 kDa, about 3.9 kDa, about 4.07 kDa, about 5.22 kDa, about 10 kDa, or about 50 kDa.

In embodiments, the polydispersity of the dehydrogenated polyethylene is at least about 1.001, at least about 1.01, at least about 1.05, at least about 1.1, at least about 1.25, at least about 1.5, at least about 1.6, at least about 1.7, at least about 1.75, at least about 1.8, at least about 1.84, at least about 1.9, at least about 2, at least about 2.1, at least about 2.25, at least about 2.5, at least about 2.75, at least about 3, at least about 3.25, at least about 3.35, at least about 3.4, at least about 3.45, at least about 3.48, at least about 3.5, at least about 3.55, at least about 3.75, at least about 4, at least about 4.25, at least about 4.5, at least about 4.75, at least about 5, or more.

In embodiments, the polydispersity of the dehydrogenated polyethylene is at most about 1.001, at most about 1.01, at most about 1.05, at most about 1.1, at most about 1.25, at most about 1.5, at most about 1.6, at most about 1.7, at most about 1.75, at most about 1.8, at most about 1.84, at most about 1.9, at most about 2, at most about 2.1, at most about 2.25, at most about 2.5, at most about 2.75, at most about 3, at most about 3.25, at most about 3.35, at most about 3.4, at most about 3.45, at most about 3.48, at most about 3.5, at most about 3.55, at most about 3.75, at most about 4, at most about 4.25, at most about 4.5, at most about 4.75, at most about 5, or less.

In embodiments, the polydispersity of the dehydrogenated polyethylene is about 1.1 to about 8. In embodiments, the polydispersity of the dehydrogenated polyethylene is at least about 1.1. In embodiments, the polydispersity of the dehydrogenated polyethylene is at most about 8. In embodiments, the polydispersity of the dehydrogenated polyethylene is about 1.1 to about 1.3, about 1.1 to about 1.5, about 1.1 to about 1.7, about 1.1 to about 1.8, about 1.1 to about 1.84, about 1.1 to about 1.9, about 1.1 to about 2.5, about 1.1 to about 3.25, about 1.1 to about 3.48, about 1.1 to about 5, about 1.1 to about 8, about 1.3 to about 1.5, about 1.3 to about 1.7, about 1.3 to about 1.8, about 1.3 to about 1.84, about 1.3 to about 1.9, about 1.3 to about 2.5, about 1.3 to about 3.25, about 1.3 to about 3.48, about 1.3 to about 5, about 1.3 to about 8, about 1.5 to about 1.7, about 1.5 to about 1.8, about 1.5 to about 1.84, about 1.5 to about 1.9, about 1.5 to about 2.5, about 1.5 to about 3.25, about 1.5 to about 3.48, about 1.5 to about 5, about 1.5 to about 8, about 1.7 to about 1.8, about 1.7 to about 1.84, about 1.7 to about 1.9, about 1.7 to about 2.5, about 1.7 to about 3.25, about 1.7 to about 3.48, about 1.7 to about 5, about 1.7 to about 8, about 1.8 to about 1.84, about 1.8 to about 1.9, about 1.8 to about 2.5, about 1.8 to about 3.25, about 1.8 to about 3.48, about 1.8 to about 5, about 1.8 to about 8, about 1.84 to about 1.9, about 1.84 to about 2.5, about 1.84 to about 3.25, about 1.84 to about 3.48, about 1.84 to about 5, about 1.84 to about 8, about 1.9 to about 2.5, about 1.9 to about 3.25, about 1.9 to about 3.48, about 1.9 to about 5, about 1.9 to about 8, about 2.5 to about 3.25, about 2.5 to about 3.48, about 2.5 to about 5, about 2.5 to about 8, about 3.25 to about 3.48, about 3.25 to about 5, about 3.25 to about 8, about 3.48 to about 5, about 3.48 to about 8, or about 5 to about 8. In embodiments, the polydispersity of the dehydrogenated polyethylene is about 1.1, about 1.3, about 1.5, about 1.7, about 1.8, about 1.84, about 1.9, about 2.5, about 3.25, about 3.48, about 5, or about 8.

In embodiments, a percentage of from about 0.01% to about 11% of monomer units of the substituted or unsubstituted dehydrogenated polyethylene are unsaturated. In embodiments, a percentage of from about 0.01% to about 10% of monomer units of the substituted or unsubstituted dehydrogenated polyethylene are unsaturated. In embodiments, a percentage of from about 0.01% to about 9% of monomer units of the substituted or unsubstituted dehydrogenated polyethylene are unsaturated. In embodiments, a percentage of from about 0.01% to about 8% of monomer units of the substituted or unsubstituted dehydrogenated polyethylene are unsaturated. In embodiments, a percentage of about 8% or less of monomer units of the substituted or unsubstituted dehydrogenated polyethylene are unsaturated. In embodiments, a percentage of from about 0.01% to about 1.5% of monomer units in the substituted or unsubstituted dehydrogenated polyethylene are unsaturated. In embodiments, a percentage of from about 2.4% to about 4.3% of monomer units in the substituted or unsubstituted dehydrogenated polyethylene are unsaturated. In embodiments, a percentage of from about 4.5% to about 13.2% of monomer units in the substituted or unsubstituted dehydrogenated polyethylene are unsaturated. In embodiments, a percentage of from about 1% to about 3% of monomer units in the substituted or unsubstituted dehydrogenated polyethylene are unsaturated. In embodiments, a percentage of from about 1.5% to about 3% of monomer units of the substituted or unsubstituted dehydrogenated polyethylene are unsaturated. In embodiments, a percentage of about 1.9% of monomer units of the substituted or unsubstituted dehydrogenated polyethylene are unsaturated. In embodiments, a percentage of from about 1.7% to about 2.3% of monomer units in the substituted or unsubstituted dehydrogenated polyethylene are unsaturated. In embodiments, a percentage of from about 1.7% to about 2.1% of monomer units in the substituted or unsubstituted dehydrogenated polyethylene are unsaturated. In embodiments, a percentage of from about 1.9% to about 2.1% of monomer units in the substituted or unsubstituted dehydrogenated polyethylene are unsaturated. In embodiments, a percentage of about 1.7% of monomer units in the substituted or unsubstituted dehydrogenated polyethylene are unsaturated. In embodiments, a percentage of about 1.8% of monomer units in the substituted or unsubstituted dehydrogenated polyethylene are unsaturated. In embodiments, a percentage of about 1.9% of monomer units in the substituted or unsubstituted dehydrogenated polyethylene are unsaturated. In embodiments, a percentage of about 2% of monomer units in the substituted or unsubstituted dehydrogenated polyethylene are unsaturated. In embodiments, a percentage of about 2.1% of monomer units in the substituted or unsubstituted dehydrogenated polyethylene are unsaturated. In embodiments, a percentage of about 2.2% of monomer units in the substituted or unsubstituted dehydrogenated polyethylene are unsaturated. In embodiments, a percentage of about 2.3% of monomer units in the substituted or unsubstituted dehydrogenated polyethylene are unsaturated.

In embodiments, a percentage of from 0.01% to 11% of monomer units of the substituted or unsubstituted dehydrogenated polyethylene are unsaturated. In embodiments, a percentage of from 0.01% to 10% of monomer units of the substituted or unsubstituted dehydrogenated polyethylene are unsaturated. In embodiments, a percentage of from 0.01% to 9% of monomer units of the substituted or unsubstituted dehydrogenated polyethylene are unsaturated. In embodiments, a percentage of from 0.01% to 8% of monomer units of the substituted or unsubstituted dehydrogenated polyethylene are unsaturated. In embodiments, a percentage of 8% or less of monomer units of the substituted or unsubstituted dehydrogenated polyethylene are unsaturated. In embodiments, a percentage of from 0.01% to 1.5% of monomer units in the substituted or unsubstituted dehydrogenated polyethylene are unsaturated. In embodiments, a percentage of from 2.4% to 4.3% of monomer units in the substituted or unsubstituted dehydrogenated polyethylene are unsaturated. In embodiments, a percentage of from 4.5% to 13.2% of monomer units in the substituted or unsubstituted dehydrogenated polyethylene are unsaturated. In embodiments, a percentage of from 1% to 3% of monomer units in the substituted or unsubstituted dehydrogenated polyethylene are unsaturated. In embodiments, a percentage of from 1.5% to 3% of monomer units of the substituted or unsubstituted dehydrogenated polyethylene are unsaturated. In embodiments, a percentage of 1.9% of monomer units of the substituted or unsubstituted dehydrogenated polyethylene are unsaturated. In embodiments, a percentage of from 1.7% to 2.3% of monomer units in the substituted or unsubstituted dehydrogenated polyethylene are unsaturated. In embodiments, a percentage of from 1.7% to 2.1% of monomer units in the substituted or unsubstituted dehydrogenated polyethylene are unsaturated. In embodiments, a percentage of from 1.9% to 2.1% of monomer units in the substituted or unsubstituted dehydrogenated polyethylene are unsaturated. In embodiments, a percentage of 1.7% of monomer units in the substituted or unsubstituted dehydrogenated polyethylene are unsaturated. In embodiments, a percentage of 1.8% of monomer units in the substituted or unsubstituted dehydrogenated polyethylene are unsaturated. In embodiments, a percentage of 1.9% of monomer units in the substituted or unsubstituted dehydrogenated polyethylene are unsaturated. In embodiments, a percentage of 2% of monomer units in the substituted or unsubstituted dehydrogenated polyethylene are unsaturated. In embodiments, a percentage of 2.1% of monomer units in the substituted or unsubstituted dehydrogenated polyethylene are unsaturated. In embodiments, a percentage of 2.2% of monomer units in the substituted or unsubstituted dehydrogenated polyethylene are unsaturated. In embodiments, a percentage of 2.3% of monomer units in the substituted or unsubstituted dehydrogenated polyethylene are unsaturated.

In some embodiments, the percentage of aromatic carbons in the dehydrogenated polyethylene is at least about 0%, at least about 0.0001%, at least about 0.001%, at least about 0.01%, at least about 0.02%, at least about 0.03%, at least about 0.04%, at least about 0.05%, at least about 0.06%, at least about 0.07%, at least about 0.08%, at least about 0.09, at least about 0.1%, at least about 0.11%, at least about 0.12%, at least about 0.13%, at least about 0.14%, at least about 0.15%, at least about 0.16%, at least about 0.17%, at least about 0.18%, at least about 0.19%, at least about 0.2%, at least about 0.22%, at least about 0.24%, at least about 0.26%, at least about 0.28%, at least about 0.3%, at least about 0.32%, at least about 0.33%, at least about 0.34%, at least about 0.35%, at least about 0.4%, at least about 0.5%, at least about 0.54%, at least about 0.55%, at least about 0.6%, at least about 0.7%, at least about 0.75%, at least about 0.77%, at least about 0.8%, at least about 0.9%, at least about 1%, at least about 1.1%, at least about 1.2%, at least about 1.23%, at least about 1.3%, at least about 1.39%, at least about 1.4%, at least about 1.43%, at least about 1.47%, at least about 1.5%, at least about 1.75%, at least about 2%, at least about 3%, at least about 4%, at least about 5%, at least about 6%, at least about 7%, at least about 8%, at least about 9%, at least about 10%, at least about 12.5%, at least about 15%, at least about 17.5%, at least about 20%, at least about 50%, at least about 75%, or more.

In some embodiments, the percentage of aromatic carbons in the dehydrogenated polyethylene is at most about 0%, at most about 0.0001%, at most about 0.001%, at most about 0.01%, at most about 0.02%, at most about 0.03%, at most about 0.04%, at most about 0.05%, at most about 0.06%, at most about 0.07%, at most about 0.08%, at most about 0.09, at most about 0.1%, at most about 0.11%, at most about 0.12%, at most about 0.13%, at most about 0.14%, at most about 0.15%, at most about 0.16%, at most about 0.17%, at most about 0.18%, at most about 0.19%, at most about 0.2%, at most about 0.22%, at most about 0.24%, at most about 0.26%, at most about 0.28%, at most about 0.3%, at most about 0.32%, at most about 0.33%, at most about 0.34%, at most about 0.35%, at most about 0.4%, at most about 0.5%, at most about 0.54%, at most about 0.55%, at most about 0.6%, at most about 0.7%, at most about 0.75%, at most about 0.77%, at most about 0.8%, at most about 0.9%, at most about 1%, at most about 1.1%, at most about 1.2%, at most about 1.23%, at most about 1.3%, at most about 1.39%, at most about 1.4%, at most about 1.43%, at most about 1.47%, at most about 1.5%, at most about 1.75%, at most about 2%, at most about 3%, at most about 4%, at most about 5%, at most about 6%, at most about 7%, at most about 8%, at most about 9%, at most about 10%, at most about 12.5%, at most about 15%, at most about 17.5%, at most about 20%, at most about 50%, at most about 75%, or more.

In some embodiments, the percentage of aromatic carbons in the dehydrogenated polyethylene is about 0.001% to about 10%. In some embodiments, the percentage of aromatic carbons in the dehydrogenated polyethylene is at least about 0.001%. In some embodiments, the percentage of aromatic carbons in the dehydrogenated polyethylene is at most about 10%. In some embodiments, the percentage of aromatic carbons in the dehydrogenated polyethylene is about 0.001% to about 0.01%, about 0.001% to about 0.02%, about 0.001% to about 0.04%, about 0.001% to about 0.08%, about 0.001% to about 0.09%, about 0.001% to about 0.1%, about 0.001% to about 0.11%, about 0.001% to about 0.33%, about 0.001% to about 0.47%, about 0.001% to about 1.47%, about 0.001% to about 10%, about 0.01% to about 0.02%, about 0.01% to about 0.04%, about 0.01% to about 0.08%, about 0.01% to about 0.09%, about 0.01% to about 0.1%, about 0.01% to about 0.11%, about 0.01% to about 0.33%, about 0.01% to about 0.47%, about 0.01% to about 1.47%, about 0.01% to about 10%, about 0.02% to about 0.04%, about 0.02% to about 0.08%, about 0.02% to about 0.09%, about 0.02% to about 0.1%, about 0.02% to about 0.11%, about 0.02% to about 0.33%, about 0.02% to about 0.47%, about 0.02% to about 1.47%, about 0.02% to about 10%, about 0.04% to about 0.08%, about 0.04% to about 0.09%, about 0.04% to about 0.1%, about 0.04% to about 0.11%, about 0.04% to about 0.33%, about 0.04% to about 0.47%, about 0.04% to about 1.47%, about 0.04% to about 10%, about 0.08% to about 0.09%, about 0.08% to about 0.1%, about 0.08% to about 0.11%, about 0.08% to about 0.33%, about 0.08% to about 0.47%, about 0.08% to about 1.47%, about 0.08% to about 10%, about 0.09% to about 0.1%, about 0.09% to about 0.11%, about 0.09% to about 0.33%, about 0.09% to about 0.47%, about 0.09% to about 1.47%, about 0.09% to about 10%, about 0.1% to about 0.11%, about 0.1% to about 0.33%, about 0.1% to about 0.47%, about 0.1% to about 1.47%, about 0.1% to about 10%, about 0.11% to about 0.33%, about 0.11% to about 0.47%, about 0.11% to about 1.47%, about 0.11% to about 10%, about 0.33% to about 0.47%, about 0.33% to about 1.47%, about 0.33% to about 10%, about 0.47% to about 1.47%, about 0.47% to about 10%, or about 1.47% to about 10%. In some embodiments, the percentage of aromatic carbons in the dehydrogenated polyethylene is about 0.001%, about 0.01%, about 0.02%, about 0.04%, about 0.08%, about 0.09%, about 0.1%, about 0.11%, about 0.33%, about 0.47%, about 1.47%, or about 10%.

In embodiments, the distribution of unsaturated monomer units in the substituted or unsubstituted dehydrogenated polyethylene is irregular.

In embodiments, the dehydrogenated polyethylene is produced from an HDPE precursor. In embodiments, the HDPE precursor has a number average molecular weight of at least about 0.5 kDa, at least about 1 kDa, at least about 2 kDa, at least about 5 kDa, at least about 10 kDa, at least about 15 kDa, at least about 20 kDa, at least about 22.5 kDa, at least about 25 kDa, at least about 26.1 kDa, at least about 37.5 kDa, at least about 30 kDa, at least about 32.5 kDa, at least about 35 kDa, at least about 37.5 kDa, at least about 40 kDa, at least about 42.5 kDa, at least about 45 kDa, at least about 50 kDa, at least about 55 kDa, at least about 60 kDa, at least about 65 kDa, at least about 75 kDa, at least about 90 kDa, at least about 100 kDa, or more.

In embodiments, the HDPE precursor has a number average molecular weight of at most about 0.5 kDa, at most about 1 kDa, at most about 2 kDa, at most about 5 kDa, at most about 10 kDa, at most about 15 kDa, at most about 20 kDa, at most about 22.5 kDa, at most about 25 kDa, at most about 26.1 kDa, at most about 37.5 kDa, at most about 30 kDa, at most about 32.5 kDa, at most about 35 kDa, at most about 37.5 kDa, at most about 40 kDa, at most about 42.5 kDa, at most about 45 kDa, at most about 50 kDa, at most about 55 kDa, at most about 60 kDa, at most about 65 kDa, at most about 75 kDa, at most about 90 kDa, at most about 100 kDa, or less.

In embodiments, the number average molecular weight of the HDPE precursor is about 0.5 kDa to about 100 kDa. In embodiments, the number average molecular weight of the HDPE precursor is at least about 0.5 kDa. In embodiments, the number average molecular weight of the HDPE precursor is at most about 100 kDa. In embodiments, the number average molecular weight of the HDPE precursor is about 0.5 kDa to about 1 kDa, about 0.5 kDa to about 5 kDa, about 0.5 kDa to about 10 kDa, about 0.5 kDa to about 15 kDa, about 0.5 kDa to about 20 kDa, about 0.5 kDa to about 25 kDa, about 0.5 kDa to about 26.1 kDa, about 0.5 kDa to about 30 kDa, about 0.5 kDa to about 35 kDa, about 0.5 kDa to about 50 kDa, about 0.5 kDa to about 100 kDa, about 1 kDa to about 5 kDa, about 1 kDa to about 10 kDa, about 1 kDa to about 15 kDa, about 1 kDa to about 20 kDa, about 1 kDa to about 25 kDa, about 1 kDa to about 26.1 kDa, about 1 kDa to about 30 kDa, about 1 kDa to about 35 kDa, about 1 kDa to about 50 kDa, about 1 kDa to about 100 kDa, about 5 kDa to about 10 kDa, about 5 kDa to about 15 kDa, about 5 kDa to about 20 kDa, about 5 kDa to about 25 kDa, about 5 kDa to about 26.1 kDa, about 5 kDa to about 30 kDa, about 5 kDa to about 35 kDa, about 5 kDa to about 50 kDa, about 5 kDa to about 100 kDa, about 10 kDa to about 15 kDa, about 10 kDa to about 20 kDa, about 10 kDa to about 25 kDa, about 10 kDa to about 26.1 kDa, about 10 kDa to about 30 kDa, about 10 kDa to about 35 kDa, about 10 kDa to about 50 kDa, about 10 kDa to about 100 kDa, about 15 kDa to about 20 kDa, about 15 kDa to about 25 kDa, about 15 kDa to about 26.1 kDa, about 15 kDa to about 30 kDa, about 15 kDa to about 35 kDa, about 15 kDa to about 50 kDa, about 15 kDa to about 100 kDa, about 20 kDa to about 25 kDa, about 20 kDa to about 26.1 kDa, about 20 kDa to about 30 kDa, about 20 kDa to about 35 kDa, about 20 kDa to about 50 kDa, about 20 kDa to about 100 kDa, about 25 kDa to about 26.1 kDa, about 25 kDa to about 30 kDa, about 25 kDa to about 35 kDa, about 25 kDa to about 50 kDa, about 25 kDa to about 100 kDa, about 26.1 kDa to about 30 kDa, about 26.1 kDa to about 35 kDa, about 26.1 kDa to about 50 kDa, about 26.1 kDa to about 100 kDa, about 30 kDa to about 35 kDa, about 30 kDa to about 50 kDa, about 30 kDa to about 100 kDa, about 35 kDa to about 50 kDa, about 35 kDa to about 100 kDa, or about 50 kDa to about 100 kDa. In embodiments, the number average molecular weight of the HDPE precursor is about 0.5 kDa, about 1 kDa, about 5 kDa, about 10 kDa, about 15 kDa, about 20 kDa, about 25 kDa, about 26.1 kDa, about 30 kDa, about 35 kDa, about 50 kDa, or about 100 kDa.

In embodiments, the dehydrogenated polyethylene is produced from an LDPE precursor. In embodiments, the LDPE precursor has a number average molecular weight of at least about 0.5 kDa, at least about 1 kDa, at least about 2 kDa, at least about 5 kDa, at least about 10 kDa, at least about 15 kDa, at least about 20 kDa, at least about 22.5 kDa, at least about 25 kDa, at least about 26.1 kDa, at least about 30 kDa, at least about 32.5 kDa, at least about 35.1 kDa, at least about 37.5 kDa, at least about 30 kDa, at least about 32.5 kDa, at least about 35 kDa, at least about 37.5 kDa, at least about 40 kDa, at least about 42.5 kDa, at least about 45 kDa, at least about 50 kDa, at least about 55 kDa, at least about 60 kDa, at least about 65 kDa, at least about 75 kDa, at least about 90 kDa, at least about 100 kDa, or more.

In embodiments, the LDPE precursor has a number average molecular weight of at most about 0.5 kDa, at most about 1 kDa, at most about 2 kDa, at most about 5 kDa, at most about 10 kDa, at most about 15 kDa, at most about 20 kDa, at most about 22.5 kDa, at most about 25 kDa, at most about 26.1 kDa, at most about 30 kDa, at most about 32.5 kDa, at most about 35.1 kDa, at most about 37.5 kDa, at most about 30 kDa, at most about 32.5 kDa, at most about 35 kDa, at most about 37.5 kDa, at most about 40 kDa, at most about 42.5 kDa, at most about 45 kDa, at most about 50 kDa, at most about 55 kDa, at most about 60 kDa, at most about 65 kDa, at most about 75 kDa, at most about 90 kDa, at most about 100 kDa, or less.

In embodiments, the number average molecular weight of the LDPE precursor is about 1 kDa to about 100 kDa. In embodiments, the number average molecular weight of the LDPE precursor is at least about 1 kDa. In embodiments, the number average molecular weight of the LDPE precursor is at most about 100 kDa. In embodiments, the number average molecular weight of the LDPE precursor is about 1 kDa to about 5 kDa, about 1 kDa to about 10 kDa, about 1 kDa to about 15 kDa, about 1 kDa to about 20 kDa, about 1 kDa to about 25 kDa, about 1 kDa to about 30 kDa, about 1 kDa to about 32.5 kDa, about 1 kDa to about 35.1 kDa, about 1 kDa to about 37.5 kDa, about 1 kDa to about 50 kDa, about 1 kDa to about 100 kDa, about 5 kDa to about 10 kDa, about 5 kDa to about 15 kDa, about 5 kDa to about 20 kDa, about 5 kDa to about 25 kDa, about 5 kDa to about 30 kDa, about 5 kDa to about 32.5 kDa, about 5 kDa to about 35.1 kDa, about 5 kDa to about 37.5 kDa, about 5 kDa to about 50 kDa, about 5 kDa to about 100 kDa, about 10 kDa to about 15 kDa, about 10 kDa to about 20 kDa, about 10 kDa to about 25 kDa, about 10 kDa to about 30 kDa, about 10 kDa to about 32.5 kDa, about 10 kDa to about 35.1 kDa, about 10 kDa to about 37.5 kDa, about 10 kDa to about 50 kDa, about 10 kDa to about 100 kDa, about 15 kDa to about 20 kDa, about 15 kDa to about 25 kDa, about 15 kDa to about 30 kDa, about 15 kDa to about 32.5 kDa, about 15 kDa to about 35.1 kDa, about 15 kDa to about 37.5 kDa, about 15 kDa to about 50 kDa, about 15 kDa to about 100 kDa, about 20 kDa to about 25 kDa, about 20 kDa to about 30 kDa, about 20 kDa to about 32.5 kDa, about 20 kDa to about 35.1 kDa, about 20 kDa to about 37.5 kDa, about 20 kDa to about 50 kDa, about 20 kDa to about 100 kDa, about 25 kDa to about 30 kDa, about 25 kDa to about 32.5 kDa, about 25 kDa to about 35.1 kDa, about 25 kDa to about 37.5 kDa, about 25 kDa to about 50 kDa, about 25 kDa to about 100 kDa, about 30 kDa to about 32.5 kDa, about 30 kDa to about 35.1 kDa, about 30 kDa to about 37.5 kDa, about 30 kDa to about 50 kDa, about 30 kDa to about 100 kDa, about 32.5 kDa to about 35.1 kDa, about 32.5 kDa to about 37.5 kDa, about 32.5 kDa to about 50 kDa, about 32.5 kDa to about 100 kDa, about 35.1 kDa to about 37.5 kDa, about 35.1 kDa to about 50 kDa, about 35.1 kDa to about 100 kDa, about 37.5 kDa to about 50 kDa, about 37.5 kDa to about 100 kDa, or about 50 kDa to about 100 kDa. In embodiments, the number average molecular weight of the LDPE precursor is about 1 kDa, about 5 kDa, about 10 kDa, about 15 kDa, about 20 kDa, about 25 kDa, about 30 kDa, about 32.5 kDa, about 35.1 kDa, about 37.5 kDa, about 50 kDa, or about 100 kDa.

In embodiments, the dehydrogenated polyethylene is produced from LLDPE.

In an aspect, provided herein is a dehydrogenated polypropylene. In some embodiments, the dehydrogenated polypropylene is substituted or unsubstituted. In an aspect the dehydrogenated polyethylene is produced by dehydrogenation of polypropylene. Alternatively, in some embodiments, the dehydrogenated polypropylene is not produced by dehydrogenation of polypropylene. In some embodiments, the dehydrogenated polypropylene is produced by installation of a leaving group on polypropylene followed by elimination. In some embodiments, the dehydrogenated polypropylene is produced by fragmentation of a precursor into fragments (e.g., cracking, pyrolysis, fluid catalytic cracking, catalytic cracking). In some embodiments, the dehydrogenated polypropylene comprises an olefin moiety. In some embodiments, the dehydrogenated polypropylene comprises a trisubstituted olefin. In some embodiments, the dehydrogenated polypropylene comprises a disubstituted olefin. In some embodiments, the dehydrogenated polypropylene comprises a 1,1-disubstituted olefin. In some embodiments, the dehydrogenated polypropylene comprises a 1,1-disubstituted olefin or a trisubstituted olefin. In some embodiments, the dehydrogenated polypropylene comprises a 1,1-disubstituted olefin or a trisubstituted olefin. In some embodiments, the dehydrogenated polypropylene comprises a 1,1-disubstituted olefin and a trisubstituted olefin.

In some embodiments, the percentage of olefinic carbons in the dehydrogenated polypropylene is at least about 0.01%, at least about 0.02%, at least about 0.05%, at least about 0.07%, at least about 0.1%, at least about 0.2%, at least about 0.5%, at least about 0.75%, at least about 1%%, at least about 1.51%, at least about 2%, at least about 3%, at least about 4%, at least about 5%, at least about 5.33%, at least about 6%, at least about 7%, at least about 7.5%, at least about 7.97%, at least about 8%, at least about 8.5%, at least about 8.85%, at least about 9%, at least about 9.5%, at least about 10%, at least about 10.09%, at least about 10.5%, at least about 11%, at least about 11.5%, at least about 12%, at least about 12%, at least about 13%, at least about 14%, at least about 15%, at least about 17.5%, at least about 20%, at least about 30%, at least about 40%, or more.

In some embodiments, the percentage of olefinic carbons in the dehydrogenated polypropylene is at most about 0.01%, at most about 0.02%, at most about 0.05%, at most about 0.07%, at most about 0.1%, at most about 0.2%, at most about 0.5%, at most about 0.75%, at most about 1%, at most about 1.51%, at most about 2%, at most about 3%, at most about 4%, at most about 5%, at most about 5.33%, at most about 6%, at most about 7%, at most about 7.5%, at most about 7.97%, at most about 8%, at most about 8.5%, at most about 8.85%, at most about 9%, at most about 9.5%, at most about 10%, at most about 10.09%, at most about 10.5%, at most about 11%, at most about 11.5%, at most about 12%, at most about 12%, at most about 13%, at most about 14%, at most about 15%, at most about 17.5%, at most about 20%, at most about 30%, at most about 40%, or less.

In some embodiments, the percentage of olefinic carbons in the dehydrogenated polypropylene is about 0.1% to about 25%. In some embodiments, the percentage of olefinic carbons in the dehydrogenated polypropylene is at least about 0.1%. In some embodiments, the percentage of olefinic carbons in the dehydrogenated polypropylene is at most about 25%. In some embodiments, the percentage of olefinic carbons in the dehydrogenated polypropylene is about 0.1% to about 1%, about 0.1% to about 1.51%, about 0.1% to about 3%, about 0.1% to about 4%, about 0.1% to about 5.33%, about 0.1% to about 6.5%, about 0.1% to about 7.97%, about 0.1% to about 8.85%, about 0.1% to about 10.09%, about 0.1% to about 15%, about 0.1% to about 25%, about 1% to about 1.51%, about 1% to about 3%, about 1% to about 4%, about 1% to about 5.33%, about 1% to about 6.5%, about 1% to about 7.97%, about 1% to about 8.85%, about 1% to about 10.09%, about 1% to about 15%, about 1% to about 25%, about 1.51% to about 3%, about 1.51% to about 4%, about 1.51% to about 5.33%, about 1.51% to about 6.5%, about 1.51% to about 7.97%, about 1.51% to about 8.85%, about 1.51% to about 10.09%, about 1.51% to about 15%, about 1.51% to about 25%, about 3% to about 4%, about 3% to about 5.33%, about 3% to about 6.5%, about 3% to about 7.97%, about 3% to about 8.85%, about 3% to about 10.09%, about 3% to about 15%, about 3% to about 25%, about 4% to about 5.33%, about 4% to about 6.5%, about 4% to about 7.97%, about 4% to about 8.85%, about 4% to about 10.09%, about 4% to about 15%, about 4% to about 25%, about 5.33% to about 6.5%, about 5.33% to about 7.97%, about 5.33% to about 8.85%, about 5.33% to about 10.09%, about 5.33% to about 15%, about 5.33% to about 25%, about 6.5% to about 7.97%, about 6.5% to about 8.85%, about 6.5% to about 10.09%, about 6.5% to about 15%, about 6.5% to about 25%, about 7.97% to about 8.85%, about 7.97% to about 10.09%, about 7.97% to about 15%, about 7.97% to about 25%, about 8.85% to about 10.09%, about 8.85% to about 15%, about 8.85% to about 25%, about 10.09% to about 15%, about 10.09% to about 25%, or about 15% to about 25%. In some embodiments, the percentage of olefinic carbons in the dehydrogenated polypropylene is about 0.1%, about 1%, about 1.51%, about 3%, about 4%, about 5.33%, about 6.5%, about 7.97%, about 8.85%, about 10.09%, about 15%, or about 25%.

In some embodiments, the percentage of monomer units comprising internal (e.g., trisubstituted) olefins in the dehydrogenated polypropylene is at least about 0.01%, at least about 0.02%, at least about 0.05%, at least about 0.07%, at least about 0.1%, at least about 0.2%, at least about 0.5%, at least about 0.75%, at least about 1%%, at least about 1.51%, at least about 2%, at least about 3%, at least about 4%, at least about 5%, at least about 5.92%, at least about 6%, at least about 6.7%, at least about 7%, at least about 7.5%, at least about 8%, at least about 8.5%, at least about 9%, at least about 9.23%, at least about 9.5%, at least about 10%, at least about 10.5%, at least about 11%, at least about 11.5%, at least about 12%, at least about 12%, at least about 13%, at least about 14%, at least about 15%, at least about 17.5%, at least about 20%, at least about 30%, at least about 40%, or more.

Alternatively, or in addition, i6.n some embodiments, the percentage of monomer units comprising internal (e.g., trisubstituted) olefins in the dehydrogenated polypropylene is at most about 0.01%, at most about 0.02%, at most about 0.05%, at most about 0.07%, at most about 0.1%, at most about 0.2%, at most about 0.5%, at most about 0.75%, at most about 1%%, at most about 1.51%, at most about 2%, at most about 3%, at most about 4%, at most about 5%, at most about 5.92%, at most about 6%, at most about 6.7%, at most about 7%, at most about 7.5%, at most about 8%, at most about 8.5%, at most about 9%, at most about 9.23%, at most about 9.5%, at most about 10%, at most about 10.5%, at most about 11%, at most about 11.5%, at most about 12%, at most about 12%, at most about 13%, at most about 14%, at most about 15%, at most about 17.5%, at most about 20%, at most about 30%, at most about 40%, or less.

In some embodiments, the percentage of monomer units comprising internal (e.g., trisubstituted) olefins in the dehydrogenated polypropylene is about 0.1% to about 30%. In some embodiments, the percentage of monomer units comprising internal (e.g., trisubstituted) olefins in the dehydrogenated polypropylene is at least about 0.1%. In some embodiments, the percentage of monomer units comprising internal (e.g., trisubstituted) olefins in the dehydrogenated polypropylene is at most about 30%. In some embodiments, the percentage of monomer units comprising internal (e.g., trisubstituted) olefins in the dehydrogenated polypropylene is about 0.1% to about 1%, about 0.1% to about 2%, about 0.1% to about 3%, about 0.1% to about 5%, about 0.1% to about 5.92%, about 0.1% to about 6.7%, about 0.1% to about 7.5%, about 0.1% to about 8.5%, about 0.1% to about 9.23%, about 0.1% to about 15%, about 0.1% to about 30%, about 1% to about 2%, about 1% to about 3%, about 1% to about 5%, about 1% to about 5.92%, about 1% to about 6.7%, about 1% to about 7.5%, about 1% to about 8.5%, about 1% to about 9.23%, about 1% to about 15%, about 1% to about 30%, about 2% to about 3%, about 2% to about 5%, about 2% to about 5.92%, about 2% to about 6.7%, about 2% to about 7.5%, about 2% to about 8.5%, about 2% to about 9.23%, about 2% to about 15%, about 2% to about 30%, about 3% to about 5%, about 3% to about 5.92%, about 3% to about 6.7%, about 3% to about 7.5%, about 3% to about 8.5%, about 3% to about 9.23%, about 3% to about 15%, about 3% to about 30%, about 5% to about 5.92%, about 5% to about 6.7%, about 5% to about 7.5%, about 5% to about 8.5%, about 5% to about 9.23%, about 5% to about 15%, about 5% to about 30%, about 5.92% to about 6.7%, about 5.92% to about 7.5%, about 5.92% to about 8.5%, about 5.92% to about 9.23%, about 5.92% to about 15%, about 5.92% to about 30%, about 6.7% to about 7.5%, about 6.7% to about 8.5%, about 6.7% to about 9.23%, about 6.7% to about 15%, about 6.7% to about 30%, about 7.5% to about 8.5%, about 7.5% to about 9.23%, about 7.5% to about 15%, about 7.5% to about 30%, about 8.5% to about 9.23%, about 8.5% to about 15%, about 8.5% to about 30%, about 9.23% to about 15%, about 9.23% to about 30%, or about 15% to about 30%. In some embodiments, the percentage of monomer units comprising internal (e.g., trisubstituted) olefins in the dehydrogenated polypropylene is about 0.1%, about 1%, about 2%, about 3%, about 5%, about 5.92%, about 6.7%, about 7.5%, about 8.5%, about 9.23%, about 15%, or about 30%.

In some embodiments, the percentage of monomer units comprising terminal (e.g., 1,1-disubstituted) olefins in the dehydrogenated polypropylene is at least about 0.01%, at least about 0.02%, at least about 0.05%, at least about 0.07%, at least about 0.1%, at least about 0.2%, at least about 0.5%, at least about 0.75%, at least about 0.86%, at least about 1%, at least about 1.25%, at least about 1.5%, at least about 1.75%, at least about 2%, at least about 2.05%, at least about 2.1%, at least about 2.15%, at least about 2.25%, at least about 2.5%, at least about 2.75%, at least about 3%, at least about 4%, at least about 5%, at least about 6%, at least about 7%, at least about 7.5%, at least about 8%, at least about 8.5%, at least about 9%, at least about 9.5%, at least about 10%, at least about 10.5%, at least about 11%, at least about 11.5%, at least about 12%, at least about 12%, at least about 13%, at least about 14%, at least about 15%, at least about 17.5%, at least about 20%, at least about 30%, at least about 40%, or more.

In some embodiments, the percentage of monomer units comprising terminal (e.g., 1,1-disubstituted) olefins in the dehydrogenated polypropylene is at most about 0.01%, at most about 0.02%, at most about 0.05%, at most about 0.07%, at most about 0.1%, at most about 0.2%, at most about 0.5%, at most about 0.75%, at most about 0.86%, at most about 1%, at most about 1.25%, at most about 1.5%, at most about 1.75%, at most about 2%, at most about 2.05%, at most about 2.1%, at most about 2.15%, at most about 2.25%, at most about 2.5%, at most about 2.75%, at most about 3%, at most about 4%, at most about 5%, at most about 6%, at most about 7%, at most about 7.5%, at most about 8%, at most about 8.5%, at most about 9%, at most about 9.5%, at most about 10%, at most about 10.5%, at most about 11%, at most about 11.5%, at most about 12%, at most about 12%, at most about 13%, at most about 14%, at most about 15%, at most about 17.5%, at most about 20%, at most about 30%, at most about 40%, or less.

In some embodiments, the percentage of monomer units comprising terminal (e.g., 1,1-disubstituted) olefins in the dehydrogenated polypropylene is about 0.1% to about 20%. In some embodiments, the percentage of monomer units comprising terminal (e.g., 1,1-disubstituted) olefins in the dehydrogenated polypropylene is at least about 0.1%. In some embodiments, the percentage of monomer units comprising terminal (e.g., 1,1-disubstituted) olefins in the dehydrogenated polypropylene is at most about 20%. In some embodiments, the percentage of monomer units comprising terminal (e.g., 1,1-disubstituted) olefins in the dehydrogenated polypropylene is about 0.1% to about 0.5%, about 0.1% to about 0.86%, about 0.1% to about 1%, about 0.1% to about 1.5%, about 0.1% to about 1.75%, about 0.1% to about 2.05%, about 0.1% to about 2.15%, about 0.1% to about 2.5%, about 0.1% to about 3%, about 0.1% to about 5%, about 0.1% to about 20%, about 0.5% to about 0.86%, about 0.5% to about 1%, about 0.5% to about 1.5%, about 0.5% to about 1.75%, about 0.5% to about 2.05%, about 0.5% to about 2.15%, about 0.5% to about 2.5%, about 0.5% to about 3%, about 0.5% to about 5%, about 0.5% to about 20%, about 0.86% to about 1%, about 0.86% to about 1.5%, about 0.86% to about 1.75%, about 0.86% to about 2.05%, about 0.86% to about 2.15%, about 0.86% to about 2.5%, about 0.86% to about 3%, about 0.86% to about 5%, about 0.86% to about 20%, about 1% to about 1.5%, about 1% to about 1.75%, about 1% to about 2.05%, about 1% to about 2.15%, about 1% to about 2.5%, about 1% to about 3%, about 1% to about 5%, about 1% to about 20%, about 1.5% to about 1.75%, about 1.5% to about 2.05%, about 1.5% to about 2.15%, about 1.5% to about 2.5%, about 1.5% to about 3%, about 1.5% to about 5%, about 1.5% to about 20%, about 1.75% to about 2.05%, about 1.75% to about 2.15%, about 1.75% to about 2.5%, about 1.75% to about 3%, about 1.75% to about 5%, about 1.75% to about 20%, about 2.05% to about 2.15%, about 2.05% to about 2.5%, about 2.05% to about 3%, about 2.05% to about 5%, about 2.05% to about 20%, about 2.15% to about 2.5%, about 2.15% to about 3%, about 2.15% to about 5%, about 2.15% to about 20%, about 2.5% to about 3%, about 2.5% to about 5%, about 2.5% to about 20%, about 3% to about 5%, about 3% to about 20%, or about 5% to about 20%. In some embodiments, the percentage of monomer units comprising terminal (e.g., 1,1-disubstituted) olefins in the dehydrogenated polypropylene is about 0.1%, about 0.5%, about 0.86%, about 1%, about 1.5%, about 1.75%, about 2.05%, about 2.15%, about 2.5%, about 3%, about 5%, or about 20%.

In some embodiments, the number average molecular weight of the substituted or unsubstituted dehydrogenated polypropylene is at least about 0.5 kDa, at least about 0.75 kDa, at least about 1 kDa, at least about 1.5 kDa, at least about 1.75 kDa, at least about 2 kDa, at least about 2.25 kDa, at least about 2.41 kDa, at least about 2.5 kDa, at least about 2.75 kDa, at least about 3 kDa, at least about 4 kDa, at least about 5 kDa, at least about 10 kDa, at least about 20 kDa, at least about 30 kDa, at least about 40 kDa, at least about 50 kDa, at least about 75 kDa, at least about 100 kDa, at least about 250 kDa, at least about 500 kDa, or more.

Alternatively, or in addition, in some embodiments, the number average molecular weight of the substituted or unsubstituted dehydrogenated polypropylene is at most about 0.5 kDa, at most about 0.75 kDa, at most about 1 kDa, at most about 1.5 kDa, at most about 1.75 kDa, at most about 2 kDa, at most about 2.25 kDa, at most about 2.41 kDa, at most about 2.5 kDa, at most about 2.75 kDa, at most about 3 kDa, at most about 4 kDa, at most about 5 kDa, at most about 10 kDa, at most about 20 kDa, at most about 30 kDa, at most about 40 kDa, at most about 50 kDa, at most about 75 kDa, at most about 100 kDa, at most about 250 kDa, at most about 500 kDa, or more.

In some embodiments, the number average molecular weight of the substituted or unsubstituted dehydrogenated polypropylene is about 0.5 kDa to about 500 kDa. In some embodiments, the number average molecular weight of the substituted or unsubstituted dehydrogenated polypropylene is at least about 0.5 kDa. In some embodiments, the number average molecular weight of the substituted or unsubstituted dehydrogenated polypropylene is at most about 500 kDa. In some embodiments, the number average molecular weight of the substituted or unsubstituted dehydrogenated polypropylene is about 0.5 kDa to about 1 kDa, about 0.5 kDa to about 1.5 kDa, about 0.5 kDa to about 1.75 kDa, about 0.5 kDa to about 2 kDa, about 0.5 kDa to about 2.25 kDa, about 0.5 kDa to about 2.41 kDa, about 0.5 kDa to about 3 kDa, about 0.5 kDa to about 5 kDa, about 0.5 kDa to about 10 kDa, about 0.5 kDa to about 50 kDa, about 0.5 kDa to about 500 kDa, about 1 kDa to about 1.5 kDa, about 1 kDa to about 1.75 kDa, about 1 kDa to about 2 kDa, about 1 kDa to about 2.25 kDa, about 1 kDa to about 2.41 kDa, about 1 kDa to about 3 kDa, about 1 kDa to about 5 kDa, about 1 kDa to about 10 kDa, about 1 kDa to about 50 kDa, about 1 kDa to about 500 kDa, about 1.5 kDa to about 1.75 kDa, about 1.5 kDa to about 2 kDa, about 1.5 kDa to about 2.25 kDa, about 1.5 kDa to about 2.41 kDa, about 1.5 kDa to about 3 kDa, about 1.5 kDa to about 5 kDa, about 1.5 kDa to about 10 kDa, about 1.5 kDa to about 50 kDa, about 1.5 kDa to about 500 kDa, about 1.75 kDa to about 2 kDa, about 1.75 kDa to about 2.25 kDa, about 1.75 kDa to about 2.41 kDa, about 1.75 kDa to about 3 kDa, about 1.75 kDa to about 5 kDa, about 1.75 kDa to about 10 kDa, about 1.75 kDa to about 50 kDa, about 1.75 kDa to about 500 kDa, about 2 kDa to about 2.25 kDa, about 2 kDa to about 2.41 kDa, about 2 kDa to about 3 kDa, about 2 kDa to about 5 kDa, about 2 kDa to about 10 kDa, about 2 kDa to about 50 kDa, about 2 kDa to about 500 kDa, about 2.25 kDa to about 2.41 kDa, about 2.25 kDa to about 3 kDa, about 2.25 kDa to about 5 kDa, about 2.25 kDa to about 10 kDa, about 2.25 kDa to about 50 kDa, about 2.25 kDa to about 500 kDa, about 2.41 kDa to about 3 kDa, about 2.41 kDa to about 5 kDa, about 2.41 kDa to about 10 kDa, about 2.41 kDa to about 50 kDa, about 2.41 kDa to about 500 kDa, about 3 kDa to about 5 kDa, about 3 kDa to about 10 kDa, about 3 kDa to about 50 kDa, about 3 kDa to about 500 kDa, about 5 kDa to about 10 kDa, about 5 kDa to about 50 kDa, about 5 kDa to about 500 kDa, about 10 kDa to about 50 kDa, about 10 kDa to about 500 kDa, or about 50 kDa to about 500 kDa. In some embodiments, the number average molecular weight of the substituted or unsubstituted dehydrogenated polypropylene is about 0.5 kDa, about 1 kDa, about 1.5 kDa, about 1.75 kDa, about 2 kDa, about 2.25 kDa, about 2.41 kDa, about 3 kDa, about 5 kDa, about 10 kDa, about 50 kDa, or about 500 kDa.

In embodiments, the substituted or unsubstituted dehydrogenated polyethylene comprises at least one —C(R¹)═C(R²)— monomer unit, wherein R¹ and R² are as described herein, including in embodiments.

In an aspect is provided a reaction vessel comprising: (a) a dehydrogenated polymer; (b) one or more isomerization catalysts; and (c) one or more metathesis catalysts. In embodiments, the reaction vessel further includes an exogenously added olefin. In embodiments, the exogenously added olefin is ethylene.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### IV. Numbered Embodiments

Embodiment 1. A method of degrading a substance, said method comprising:
(i) conducting one or more reaction(s) to form one or more olefin units in the substance;
(ii) subjecting, after step (i), to conditions for olefin metathesis; and
(iii) subjecting, after step (i), to conditions for olefin isomerization.

Embodiment 2. The method of embodiment 1, wherein step (i) comprises subjecting the substance to conditions for dehydrogenation.

Embodiment 3. A method of making a substituted or unsubstituted alkene, said method comprising subjecting a dehydrogenated polymer to conditions for metathesis and to conditions for olefin isomerization.

Embodiment 4. A method of making a substituted or unsubstituted alkene, said method comprising subjecting a first substance to conditions for olefin metathesis and to conditions for olefin isomerization; wherein:
(i) said first substance comprises at least 20 carbon atoms per molecule; or
(ii) prior to subjecting said first substance to conditions for olefin metathesis and to conditions for olefin isomerization, the method comprises subjecting a second substance to conditions for dehydrogenation, thereby forming said first substance.

Embodiment 5. The method of one of embodiments 3 to 4, wherein the substituted or unsubstituted alkene is a substituted or unsubstituted propene.

Embodiment 6. The method of one of embodiments 4 to 5, wherein said first substance is a substituted or unsubstituted dehydrogenated polyethylene.

Embodiment 7. A method of making an alkene, said method comprising mixing a dehydrogenated polymer with one or more olefin metathesis reagent(s) and one or more isomerization reagent(s); wherein:
(i) the dehydrogenated polymer comprises at least 20 carbon atoms; or
(ii) prior to said mixing, the method comprises mixing a polymer with one or more dehydrogenating reagent(s), thereby forming said dehydrogenated polymer.

Embodiment 8. A method of making a dehydrogenated polymer, said method comprising mixing a polymer with one or more dehydrogenating reagent(s), wherein
(i) a percentage of from 0.01% to 1.5% or from 1.7% to 2.3% or from 2.4% to 4.3% or 4.5% to 13.2% of monomer units in the dehydrogenated polymer are unsaturated;
(ii) the dehydrogenated polymer has a number average molecular weight of about 2,200 Da or greater;
(iii) the dehydrogenated polymer has a number average molecular weight of about 2,200 Da to about 6,500 Da and a percentage of from 0.01% to 10% of monomer units are unsaturated; or
(iv) the dehydrogenated polymer has a number average molecular weight of about 1,000 Da to about 500,000 Da and a percentage of from 0.01% to 1.5% or from 1.7% to 2.3% or from 2.4% to 4.3% or from 4.5% to 10% of monomer units in the dehydrogenated polymer are unsaturated.

Embodiment 9. The method of embodiment 8, wherein the dehydrogenated polymer has a number average molecular weight of from about 2,200 Da to about 6,500 Da or from about 6,700 Da to about 15,000 Da.

Embodiment 10. The method of embodiment 8, wherein the dehydrogenated polymer has a number average molecular weight of from about 2,200 Da to about 6,500 Da.

Embodiment 11. The method of one of embodiments 8 to 10, further comprising mixing the dehydrogenated polymer with one or more olefin metathesis reagent(s) and one or more isomerization reagent(s), thereby forming an alkene.

Embodiment 12. The method of one of embodiments 7 to 11, wherein the dehydrogenated polymer is a substituted or unsubstituted dehydrogenated polyethylene.

Embodiment 13. The method of one of embodiments 7 to 11, wherein the alkene is a substituted or unsubstituted propene.

Embodiment 14. The method of one of embodiments 7, 11, and 12, wherein the dehydrogenated polymer, the one or more olefin metathesis reagent(s), and the one or more isomerization reagent(s) are mixed simultaneously.

Embodiment 15. The method of one of embodiments 7, 11, and 12, wherein the dehydrogenated polymer and the one or more olefin metathesis reagent(s) are mixed prior to adding the one or more isomerization reagent(s).

Embodiment 16. The method of embodiment 15, wherein the dehydrogenated polymer and one or more additional olefin metathesis reagent(s) are mixed prior to adding the one or more olefin metathesis reagent(s) and the one or more isomerization reagent(s).

Embodiment 17. The method of one of embodiments 7, 11, and 12, wherein the one or more olefin metathesis reagent(s) comprises an ethenolysis catalyst.

Embodiment 18. The method of one of embodiments 7 and 11 to 17, wherein the one or more olefin metathesis reagent(s) comprises one or more homogeneous olefin metathesis catalyst(s) or one or more heterogeneous olefin metathesis catalyst(s).

Embodiment 19. The method of embodiment 18, wherein the one or more olefin metathesis reagent(s) further comprises ethylene.

Embodiment 20. The method of embodiment 18 or 19, wherein the homogeneous olefin metathesis catalyst is

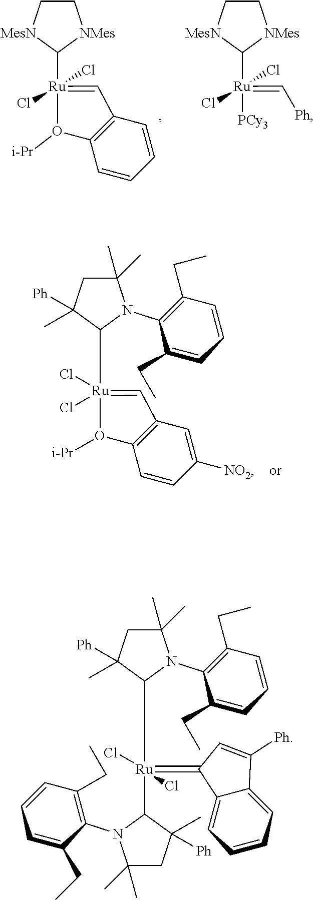

Embodiment 21. The method of embodiments 18 or 19, wherein the homogeneous olefin metathesis catalyst is

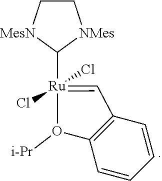

Embodiment 22. The method of embodiment 18 or 19, wherein the heterogeneous olefin metathesis catalyst is Re₂O₇/Al₂O₃ or WO₃/Al₂O₃.

Embodiment 23. The method of one of embodiments 7 and 11 to 21, wherein the one or more isomerization reagent(s) comprises one or more homogeneous isomerization catalyst(s) or one or more heterogeneous isomerization catalyst(s).

Embodiment 24. The method of embodiment 23, wherein the one or more homogeneous isomerization catalyst(s) is selected from:

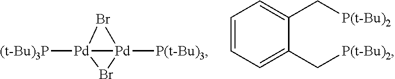

[Rh(cod)₂]BF₄, and

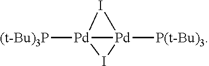

Embodiment 25. The method of embodiment 23, wherein the one or more homogeneous isomerization catalyst(s) is

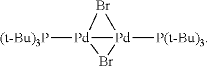

Embodiment 26. The method of embodiment 23, wherein the one or more heterogeneous isomerization catalyst(s) is selected from: Na/Al₂O₃, K/Al₂O₃, Mg/Al₂O₃, MgO, NaO₂, and KO₂.

Embodiment 27. The method of one of embodiments 7 to 25, wherein the substituted or unsubstituted polymer is a high-density polyethylene, a low-density polyethylene, or a linear low-density polyethylene, or a co-polymer thereof.

Embodiment 28. The method of one of embodiments 7 to 27, wherein the one or more dehydrogenating reagent(s) comprises a transfer dehydrogenation catalyst.

Embodiment 29. The method of one of embodiments 7 to 28, wherein the one or more dehydrogenating reagent(s) comprises a hydrogen acceptor.

Embodiment 30. The method of embodiment 29, wherein the hydrogen acceptor comprises a C—C bond, a C═C bond, a C≡C bond, an N—N bond, an N═N bond, an N≡N bond, an O—O bond, an O═O bond, a C—N bond, a C═N bond, a C≡N bond, a C—O bond, a C═O bond, a C≡O) bond, an S—S bond, a C—S bond, or a C═S bond.

Embodiment 31. The method of embodiment 29, wherein the hydrogen acceptor comprises a C═C bond.

Embodiment 32. The method of one of embodiments 29 to 31, wherein the hydrogen acceptor is ethylene, propylene, butene, tert-butylethylene, norbornene, norbornadiene, butadiene, styrene, acetylene, O₂, CO₂, CO, substituted or unsubstituted benzoquinone, or N₂.

Embodiment 33. The method of one of embodiments 7 to 32, wherein the one or more dehydrogenating reagent(s) comprises a heterogeneous dehydrogenating catalyst.

Embodiment 34. The method of embodiment 33, wherein the heterogeneous dehydrogenating catalyst comprises a plurality of metallic elements.

Embodiment 35. The method of embodiment 33, wherein the heterogeneous dehydrogenating catalyst is a bimetallic catalyst.

Embodiment 36. The method of one of embodiments 33 to 35, wherein the heterogeneous dehydrogenating catalyst is a Pt/Sn catalyst or a Pt/Zn catalyst.

Embodiment 37. The method of one of embodiments 33 to 36, wherein the heterogeneous dehydrogenating catalyst is Pt/Sn-Al₂O₃ or Pt/Zn—SiO₂.

Embodiment 38. The method of one of embodiments 7 to 32, wherein the one or more dehydrogenating reagent(s) comprises a homogeneous dehydrogenating catalyst.

Embodiment 39. The method of embodiment 38, wherein the homogeneous dehydrogenating catalyst comprises an iridium catalyst, a rhodium catalyst, or a ruthenium catalyst.

Embodiment 40. The method of embodiment 38, wherein the homogeneous dehydrogenating catalyst comprises an iridium catalyst.

Embodiment 41. The method of embodiment 38, wherein the homogeneous dehydrogenating catalyst has the formula:

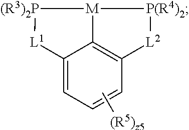

wherein
M is Ir, Rh, Ru, or Os;
L¹ and L² are independently —O—, —S—, —NR⁶—, or substituted or unsubstituted alkylene;
each R³ and R⁴ is independently substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁵ is independently halogen, —CX⁵₃, —CHX⁵₂, —CH₂X⁵, —OCX⁵₃, —OCH₂X⁵, —OCHX⁵₂, —CN, —SOₙ₅R^{5D}, —SOᵥ₅NR^{5A}R^{5B}, —NR^{5C}NR^{5A}R^{5B}, —ONR^{5A}R^{5B}, —NR^{5C}C(O)NR^{5A}R^{5B}, —N(O)ₘ₅, —NR^{5A}R^{5B}, —C(O)R^{5C}, —C(O)OR^{5C}, —OC(O)R^{5C}, —OC(O)OR^{5C}, —C(O)NR^{5A}R^{5B}, —OC(O)NR^{5A}R^{5B}, —OR^{5D}, —SR^{5D}, —NR^{5A}SO₂R^{5D}, —NR^{5A}C(O)R^{5C}, —NR^{5A}C(O)OR^{5C}, —NR^{5A}OR^{5C}, —P(O)₂(OR^{5A}), —OP(O)₂(OR^{5A}), —OP(R^{5A}) (R^{5B}), —BR^{5A}R^{5B}, —SiR^{5A}R^{5B}R^{5C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
each R⁶ is independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
each R^{5A}, R^{5B}, R^{5C}, and R^{5D} is independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or R^{5A} and R^{5B} substituents bonded to the same nitrogen atom are joined to form a substituted or unsubstituted heterocycloalkyl or substituted or unsubstituted heteroaryl;
each X⁵ is independently —Cl, —Br, —I, or —F;
n5 is 0, 1, 2, 3, or 4;
each m5 and v5 is independently 1 or 2; and
z5 is 0, 1, 2, or 3.

Embodiment 42. The method of embodiment 38, wherein the homogeneous dehydrogenating catalyst is

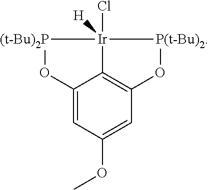

Embodiment 43. The method of one of embodiments 7 to 42, wherein a percentage of from 1% to 3% of monomer units in the dehydrogenated polymer are unsaturated.

Embodiment 44. The method of one of embodiments 7 to 42, wherein a percentage of from 1.7% to 2.1% of monomer units in the dehydrogenated polymer are unsaturated.

Embodiment 45. The method of one of embodiments 7 to 42, wherein a percentage of from 1.9% to 2.1% of monomer units in the dehydrogenated polymer are unsaturated.

Embodiment 46. The method of one of embodiments 7 to 45, wherein the dehydrogenated polymer comprises at least one —C(R¹)═C(R²)— monomer unit, wherein
R¹ is independently hydrogen, halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NR^{1C}C(O)NR^{1A}R^{1B}, —N(O)ₘ₁, —NR^{1A}R^{1B}, —C(O)R^{1C}, —C(O)OR^{1C}, —OC(O)R^{1C}, —OC(O)OR^{1C}, —C(O)NR^{1A}R^{1B}, —OC(O)NR^{1A}R^{1B}, —OR^{1D}, —SR^{1D}, —NR^{1A}SO₂R^{1D}, —NR^{1A}C(O)R^{1C}, —NR^{1A}C(O)OR^{1C}, —NR^{1A}OR^{1C}, —P(O)₂(OR^{1A}), —OP(O)₂(OR^{1A}), —BR^{1A}R^{1B}, —SiR^{1A}R^{1B}R^{1C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² is independently hydrogen, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NR^{2C}C(O)NR^{2A}R^{2B}, —N(O)ₘ₂, —NR^{2A}R^{2B}, —C(O)R^{2C}, —C(O)OR^{2C}, —OC(O)R^{2C}, —OC(O)OR^{2C}, —C(O)NR^{2A}R^{2B}, —OC(O)NR^{2A}R^{2B}, —OR^{2D}, —SR^{2D}, —NR^{2A}SO₂R^{2D}, —NR^{2A}C(O)R^{2C}, —NR^{2A}C(O)OR^{2C}, —NR^{2A}OR^{2C}, —P(O)₂(OR^{2A}), —OP(O)₂(OR^{2A}), —BR^{2A}R^{2B}, —SiR^{2A}R^{2B}R^{2C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
each R^{1A}, R^{1B}, R^{1C}, R^{1D}, R^{2A}, R^{2B}, R^{2C}, and R^{2D} is independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or R^{1A} and R^{1B} substituents bonded to the same nitrogen atom are joined to form a substituted or unsubstituted heterocycloalkyl or substituted or unsubstituted heteroaryl; or R^{2A} and R^{2B} substituents bonded to the same nitrogen atom are joined to form a substituted or unsubstituted heterocycloalkyl or substituted or unsubstituted heteroaryl;
each X¹ and X² is independently —Cl, —Br, —I, or —F;
each n1 and n2 is independently 0, 1, 2, 3, or 4; and
each m1, m2, v1, and v2 is independently 1 or 2.

Embodiment 47. The method of embodiment 46, wherein R¹ and R² are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, or substituted or unsubstituted C₁-C₂₀ alkyl.

Embodiment 48. The method of embodiment 46, wherein R¹ and R² are independently hydrogen or substituted or unsubstituted C₁-C₁₀₀₀ alkyl.

Embodiment 49. The method of embodiment 46, wherein R¹ and R² are hydrogen.

Embodiment 50. The method of one of embodiments 1 to 49, conducted under continuous flow conditions.

Embodiment 51. A substituted or unsubstituted dehydrogenated polyethylene, wherein a percentage of from 0.01% to 1.5% or from 1.7% to 2.3% or from 2.4% to 4.3% or from 4.5% to 13.2% of monomer units are unsaturated.

Embodiment 52. The dehydrogenated polyethylene of embodiment 51, having a number average molecular weight of from 1,000 Da to 500,000 Da.

Embodiment 53. A substituted or unsubstituted dehydrogenated polyethylene, having a number average molecular weight of 2,200 Da or greater.

Embodiment 54. The dehydrogenated polyethylene of embodiment 53, having a number average molecular weight of from 2,200 Da to 500,000 Da.

Embodiment 55. The dehydrogenated polyethylene of embodiment 53, having a number average molecular weight of from 2,200 Da to 6,500 Da or a number average molecular weight greater than 6,700 Da.

Embodiment 56. The dehydrogenated polyethylene of one of embodiments 53 to 55, wherein a percentage of from 0.01% to 11% of monomer units are unsaturated.

Embodiment 57. The dehydrogenated polyethylene of one of embodiments 53 to 55, wherein a percentage of from 0.01% to 10% of monomer units are unsaturated.

Embodiment 58. The dehydrogenated polyethylene of one of embodiments 53 to 55, wherein a percentage of 8% or less of monomer units are unsaturated.

Embodiment 59. The dehydrogenated polyethylene of one of embodiments 53 to 55, wherein a percentage of from 1.5% to 3% of monomer units are unsaturated.

Embodiment 60. The dehydrogenated polyethylene of one of embodiments 53 to 55, wherein a percentage of 1.9% of monomer units are unsaturated.

Embodiment 61. The dehydrogenated polyethylene of one of embodiments 51 to 60, wherein the distribution of unsaturated monomer units is irregular.

Embodiment 62. The dehydrogenated polyethylene of one of embodiments 51 to 61, comprising at least one —C(R¹)═C(R²)— monomer unit, wherein
R¹ is independently hydrogen, halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NR^{1C}C(O)NR^{1A}R^{1B}, —N(O)ₘ₁, —NR^{1A}R^{1B}, —C(O)R^{1C}, —C(O)OR^{1C}, —OC(O)R^{1C}, —OC(O)OR^{1C}, —C(O)NR^{1A}R^{1B}, —OC(O)NR^{1A}R^{1B}, —OR^{1D}, —SR^{1D}, —NR^{1A}SO₂R^{1D}, —NR^{1A}C(O)R^{1C}, —NR^{1A}C(O)OR^{1C}, —NR^{1A}OR^{1C}, —P(O)₂(OR^{1A}), —OP(O)₂(OR^{1A}), —BR^{1A}R^{1B}, —SiR^{1A}R^{1B}R^{1C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² is independently hydrogen, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NR^{2C}C(O)NR^{2A}R^{2B}, —N(O)ₘ₂, —NR^{2A}R^{2B}, —C(O)R^{2C}, —C(O)OR^{2C}, —OC(O)R^{2C}, —OC(O)OR^{2C}, —C(O)NR^{2A}R^{2B}, —OC(O)NR^{2A}R^{2B}, —OR^{2D}, —SR^{2D}, —NR^{2A}SO₂R^{2D}, —NR^{2A}C(O)R^{2C}, —NR^{2A}C(O)OR^{2C}, —NR^{2A}OR^{2C}, —P(O)₂(OR^{2A}), —OP(O)₂(OR^{2A}), —BR^{2A}R^{2B}, —SiR^{2A}R^{2B}R^{2C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
each R^{1A}, R^{1B}, R^{1C}, R^{1D}, R^{2A}, R^{2B}, R^{2C}, and R^{2D} is independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or R^{1A} and R^{1B} substituents bonded to the same nitrogen atom are joined to form a substituted or unsubstituted heterocycloalkyl or substituted or unsubstituted heteroaryl; or R^{2A} and R^{2B} substituents bonded to the same nitrogen atom are joined to form a substituted or unsubstituted heterocycloalkyl or substituted or unsubstituted heteroaryl;
each X¹ and X² is independently —Cl, —Br, —I, or —F;
each n1 and n2 is independently 0, 1, 2, 3, or 4; and
each m1, m2, v1, and v2 is independently 1 or 2.

Embodiment 63. The dehydrogenated polyethylene of embodiment 62, wherein R¹ and R² are independently hydrogen,
halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NH₂, —NHSO₂H, —NHC(O)H, —NHC(O)OH,
—NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, or substituted or unsubstituted C₁-C₁₀ alkyl.

Embodiment 64. The dehydrogenated polyethylene of embodiment 62, wherein R¹ and R² are independently hydrogen or substituted or unsubstituted C₁-C₁₀₀₀ alkyl.

Embodiment 65. The dehydrogenated polyethylene of embodiment 62, wherein R¹ and R² are hydrogen.

### EXAMPLES

### Example 1: Integrated Catalysis for Chemical Recycling of Polyethylene to Propene

Disclosed herein, inter alia, is the conversion of polyethylene to propene by integrating polyethylene dehydrogenation with tandem isomerizing ethenolysis of the resulting dehydro-polyethylene. Dehydrogenation of high-density polyethylene with an iridium-pincer complex or with Pt/Zn on silica yielded dehydrogenated material containing up to 3.2% internal olefins, and the combination of a second-generation Hoveyda-Grubbs metathesis catalyst and [PdP(^{t}Bu)₃ (μ-Br)]₂ as an isomerization catalyst selectively degraded this unsaturated polymer to propene in yields up to 80%. This process shows the feasibility of mild catalysis to deconstruct stable polyolefins.

The mechanical durability and chemical inertness of commercial plastics have led to their widespread use in everyday life, but these same properties make them difficult to recycle. Polyolefins, which account for 36% of global plastic production (1), are particularly durable and inert and are especially incompatible with traditional mechanical recycling. Only a small proportion of polyethylene (PE) waste is recovered by recycling (around 14% in 2015) (2), and the properties of the recycled materials are inferior to those of the virgin plastic. Polyolefins are prepared with molecular weights, molecular-weight distributions, and microstructures for specific applications. Thus, a mixture of polyethylenes that have been produced for diverse applications lack the properties of each individual component and possess accumulated additives and contaminants (2). Given the significant social, economic, and ecological threats from the accumulation of mixed polyolefin waste, new approaches to use this material as a carbon feedstock are needed (2-4).

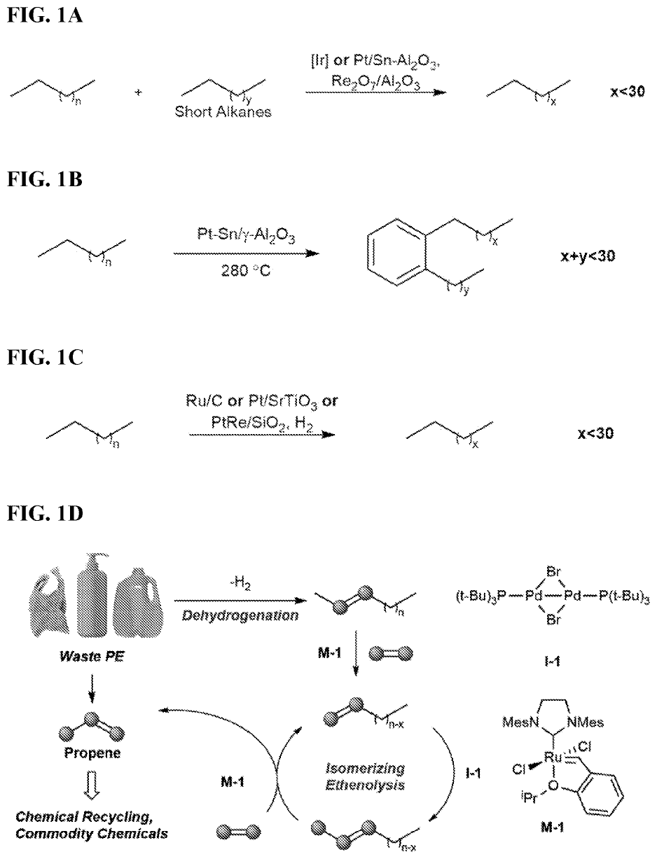

Our group (5) and others (6-8) have been investigating catalytic chemistry that could occur with these polymers containing only alkyl C—H and C—C bonds. Because the C—H bond are more reactive than the C—C bonds, the introduction of functional groups onto the polymer chain has been sought to create new properties (5, 7, 8). More challenging are methods for “chemical recycling” of polyolefins, which is the cleavage of polymer chains to regenerate monomers that could be repolymerized (6, 9). While processes are well established to cleave the carbon-oxygen bonds of polyesters (10), methods to cleave the alkyl carbon-carbon bonds of polyethylene are more limited. Pyrolysis (4), catalytic cracking (11), hydrogenolysis (12-15), tandem hydrogenolysis-aromatization (16), and alkane metathesis (17, 18) have been reported for cleavage of PE chains. However, these processes form complex mixtures of hydrocarbons which are difficult to separate and have varying values (FIG. 1A). A process is needed by which PE undergoes depolymerization to form a single product for which the demand approaches the supply of PE waste. A method for the selective production of a commodity olefin from polyethylene could thus render PE a valuable hydrocarbon feedstock.

We envisioned a cascade of catalytic processes that would introduce unsaturation to a PE chain and then unravel the chain to propene. More specifically, dehydrogenation could be used to enable subsequent C—C bond cleavage by a combination of olefin metathesis and isomerization to form propene (FIG. 1B) (19, 20). We show that the combination of dehydrogenation of high molecular-weight, high-density or low-density polyethylene and subsequent IE of the resulting crude, high-molecular-weight PE containing internal alkenes converts polyethylene to propene in yields as high as 80%. This dehydrogenation and isomerizing ethenolysis (DIE) occurs on post-consumer, in addition to laboratory-grade, polyethylene and occurs on dehydrogenated PE produced by either homogeneous or heterogeneous catalysts.

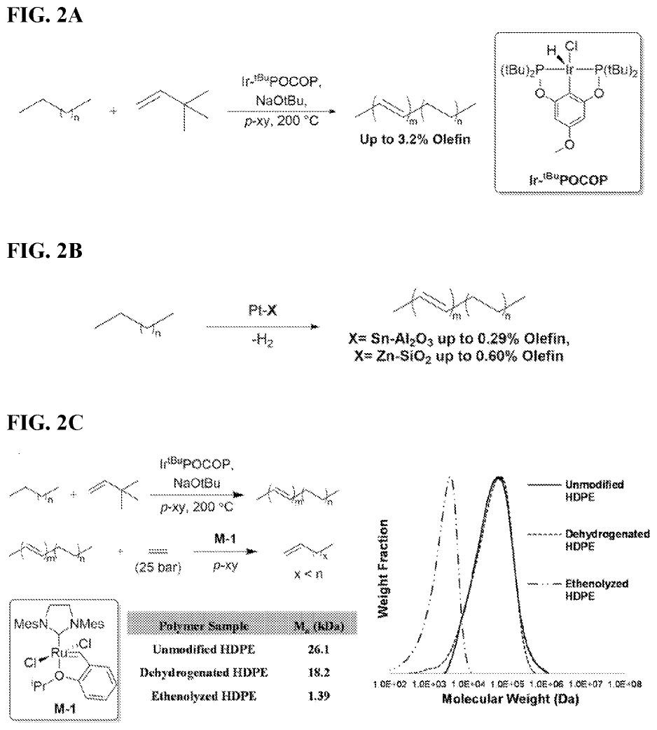

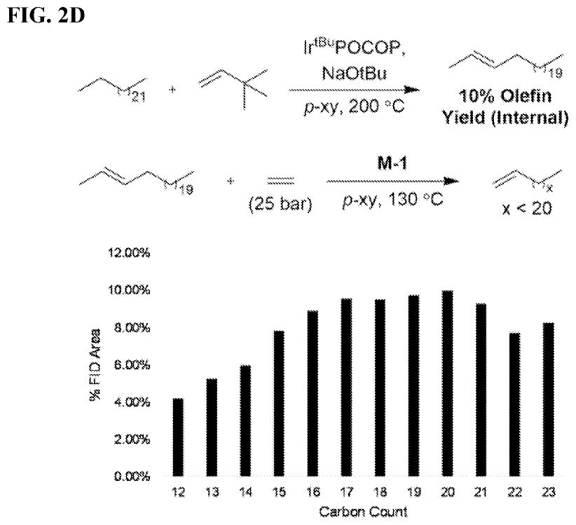

To determine whether the conversion of polyethylene to propene could be achieved by a cascade comprising dehydrogenation and isomerizing ethenolysis (DIE), a reliable dehydrogenation reaction was first needed. The dehydrogenation of PE to form unsaturated polymer has been reported using an iridium dehydrogenation catalyst, but only with material possessing an Mₙ (number-average molecular weight) of 606 to ensure solubility (21, 22). Many alkane dehydrogenation catalysts have been reported since that time, and we investigated catalysts containing symmetric ^{tBu}POCOP and related ligands (23). Dehydrogenations of laboratory-grade HDPE (Aldrich, Mₙ 26.1 kDa, Ð=3.31) and LDPE (Aldrich, Mₙ=35.1 kDa, Ð=6.54) catalyzed by Ir-^{tBu}POCOP with tert-butyl ethylene (TBE) as hydrogen acceptor in p-xylene solvent yielded unsaturated polymer containing exclusively internal olefins (up to 3.2%), along with small amounts (<0.75%) of alkylarenes (FIG. 2A), as determined by 1H NMR spectroscopy. The isomerization of terminal olefins, which might be the kinetic product, to internal olefins is well known from dehydrogenation of liquid alkanes with this catalyst (24).

Data on the dehydrogenation of polyethylenes with molecular catalysts are summarized in Table 1 below. HDPE (Commercial) refers to high density polyethylene (HDPE) sourced from Sigma Aldrich, with number average molecular weight (Mₙ) of 26.1 kDa and polydispersity (Ð) of 2.86, as measured by high temperature size-exclusion chromatography against polystyrene standards. HDPE (Can Tops) refers to previously-recycled high density polyethylene (rHDPE) sourced from commercial aluminum-can beverage packaging. HDPE (Item) refers to HDPE sourced from containers from commercial products, viz. a milk bottle and shampoo bottle. LDPE (Commercial) refers to low-density polyethylene sourced from Sigma-Aldrich, with an Mₙ of 35.1 kDa and polydispersity of 6.54. LDPE (food packaging) refers to LDPE sourced from a commercial plastic bag used for food storage.

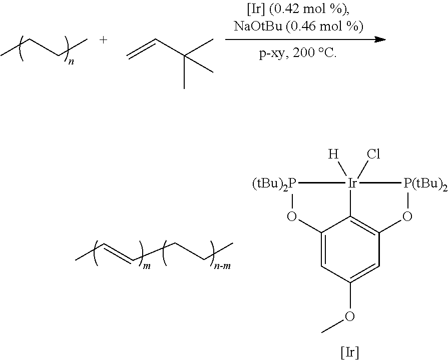

TABLE 1

| | | | Prod- | Product | Yield | Yield |
|---|---|---|---|---|---|---|
| | | Equiv | uct | Mₙ | Olefin | Arene |
| Entry | Polymer | TBE | Ð | (kDa) | (%) | (%) |
| 1 | HDPE (Commercial) | 0.020 | | | 0.62 | 0.06 |
| 2 | HDPE (Commercial) | 0.145 | 3.48 | 18.2 | 1.09 | 0.04 |
| 3 | HDPE (Commercial) | 0.200 | | | 2.38 | 0.54 |
| 4 | HDPE (Commercial) | 0.400 | | | 3.54 | 1.23 |
| 5 | HDPE (Can Tops) | 0.145 | | | 2.17 | 0.47 |
| 6 | HDPE (Milk Jug) | 0.145 | | | 1.42 | 0.77 |
| 7 | HDPE (Shampoo | 0.145 | | | 1.34 | 0.54 |
| | Bottle) | | | | | |
| 8 | LDPE (Commercial) | 0.145 | 1.84 | 5.22 | 0.81 | 0 |
| 9 | LDPE (Commercial) | 0.145 | | | 1.06 | 0.11 |
| 10 | LDPE (Commercial) | 0.183 | | | 1.76 | 1.39 |
| 11 | LDPE (Commercial) | 0.313 | | | 2.30 | 1.47 |
| 12 | LDPE (Commercial) | 0.400 | | | 2.55 | 1.43 |
| 13 | LDPE (Food | 0.145 | | | 1.46 | 0.33 |
| | Packaging) | | | | | |

Data on the dehydrogenations of polypropylene with molecular catalysts are summarized in Table 2 below. PP (Commercial) refers to isotactic polypropylene sourced from Sigma Aldrich, with an Mₙ of 68.0 kDa, as reported by the supplier.

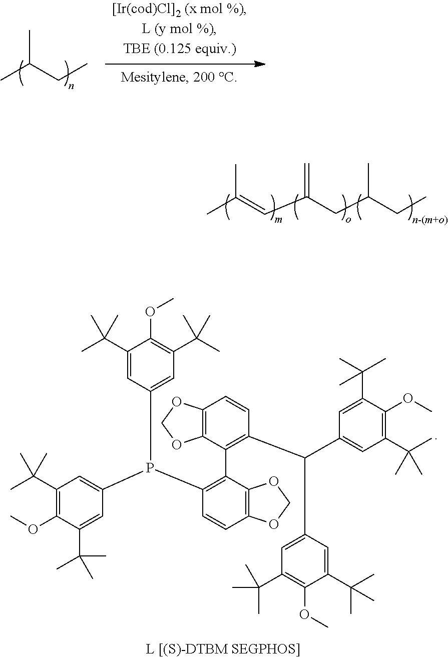

TABLE 2

| | | | | Yield 1,1- | Yield |
|---|---|---|---|---|---|
| | Equiv | [Ir(cod)Cl]₂ | L | Disubstituted | Internal |
| Entry | TBE | (mol %) | (mol %) | Olefin (%) | Olefin (%) |
| 1 | 0.125 | 0.42 | 0.84 | 2.15 | 6.70 |
| 2 | 0.125 | 0.21 | 0.42 | 2.05 | 5.92 |
| 3* | 0.125 | 0.21 | 0.42 | 0.86 | 9.23 |

*Average of 30 runs

To determine if heterogeneous catalysts also could create polyolefins containing alkene units, we investigated the dehydrogenation of polyethylene with catalysts inspired by those in UOP's Oleflex process for the dehydrogenation of long-chain paraffins. We tested the dehydrogenation of HDPE with Pt/Sn and Pt/Zn catalysts on heterogeneous supports (26). Heating of the polymer melt over such catalysts under a flow of argon at 350° C. furnished unsaturated PE containing up to 0.6% olefins, along with ~0.25% arenes (FIG. 2B). Again, all the olefin units were internal olefins.

Data on the dehydrogenation of polyolefins using multi-metallic heterogeneous catalysts are summarized in Table 3 below. All polymer labels are as specified above. Pt/Sn-γAl₂O₃ refers to a catalyst consisting of 2 wt % Pt and 4.6% Sn supported on gamma alumina. Pt/Zn—SiO₂ refers to a catalyst consisting of 0.5 wt % Pt and 0.95% Zn supported on silica. Pt/Sn/K-γAl₂O₃ refers to a catalyst consisting of 0.5 wt % Pt, 1.2% Sn, and 6.8% K supported on gamma alumina.

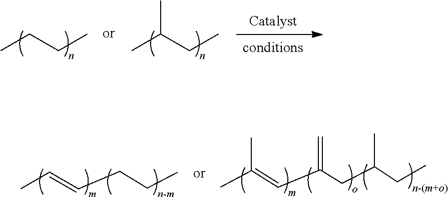

TABLE 3

| | | | | | Product | Yield | Yield |
|---|---|---|---|---|---|---|---|
| | | | Time | Temperature | Mₙ | Olefin | Arene |
| Entry | Polymer | Catalyst | (h) | (°C) | (kDa) | (%) | (%) |
| 1 | HDPE | Pt/Sn- | 18 | 350 | 1.23 | 0.56 | 0.56 |
| | (Commercial) | γAl₂O₃ | | | | | |
| 2 | HDPE | Pt/Zn—SiO₂ | 18 | 350 | 1.67 | 0.52 | 0.28 |
| | (Commercial) | | | | | | |
| 3 | HDPE | Pt/Sn/K- | 18 | 350 | 2.24 | 0.38 | 0.09 |
| | (Commercial) | γAl₂O₃ | | | | | |
| 4 | HDPE | Pt/Sn/K- | 4 | 350 | 3.90 | 0.25 | 0.05 |
| | (Commercial) | γAl₂O₃ | | | | | |
| 5 | HDPE | Pt/Sn/K- | 18 | 325 | 4.07 | 0.19 | 0.05 |
| | (Commercial) | γAl₂O₃ | | | | | |
| 6 | PP (Commercial) | Pt/Sn/K- | 18 | 300 | 2.41 | 5.33 | — |
| | | γAl₂O₃ | | | | | |
| 7 | PP (Commercial) | Pt/Sn- | 18 | 300 | — | 1.51 | — |
| | | γAl₂O₃ | | | | | |

Having demonstrated the feasibility of the dehydrogenation of high-molecular-weight PE, we tested if varying levels of alkenes could be installed into the chain. To do so, we subjected laboratory-grade HDPE to dehydrogenation with Ir-^{tBu}POCOP and varying loadings of TBE as sacrificial H₂ acceptor. These reactions produced PE with degrees of unsaturation ranging from 0.6% to 3.2%.

It was unclear if high-molecular-weight, unsaturated PE would undergo ethenolysis to form the long-chain terminal alkenes for the isomerizing ethenolysis. In the event, subjecting the unsaturated HDPE containing 1.09% olefin units to ethenolysis with 1.8 mol % M-1 resulted in a reduction of the Mₙ from 18.1 kDa to 1.6 kDa. The Mₙ of the cleaved product corresponds to an average chain length of 114 carbons (FIG. 2C). To model the process of dehydrogenation and ethenolysis on a pure reactant and to gain information on the position of the alkene units and the resulting chain lengths, we conducted these reactions on a pure, long-chain alkane, tetracosane (C₂₄H₅₀). Initial dehydrogenation of tetracosane (C₂₄H₅₀) with 1 equivalent of TBE converted about 10% of the alkane to internal tetracosenes. Subsequent ethenolysis of the reaction mixture catalyzed by M-1 under 25 bar of ethylene formed the distribution of olefins including a wide range of chain lengths shown in FIG. 2D. This set of olefins demonstrates that dehydrogenation with Ir-^{tBu}POCOP produces a distribution of internal olefins, rather than alkenes near the chain terminus.

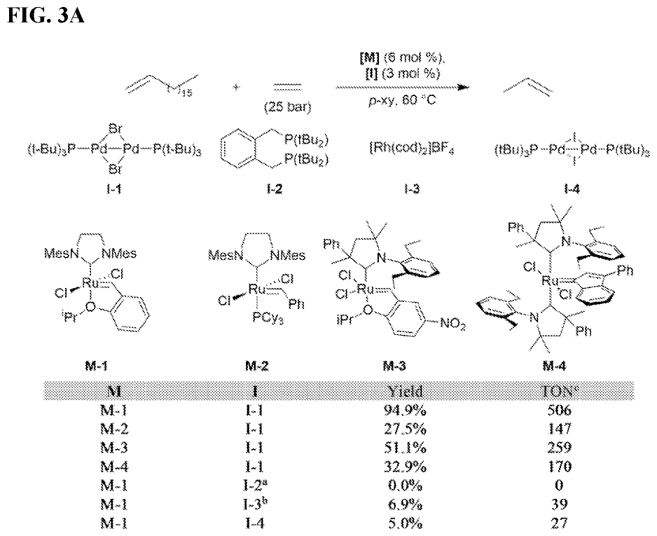

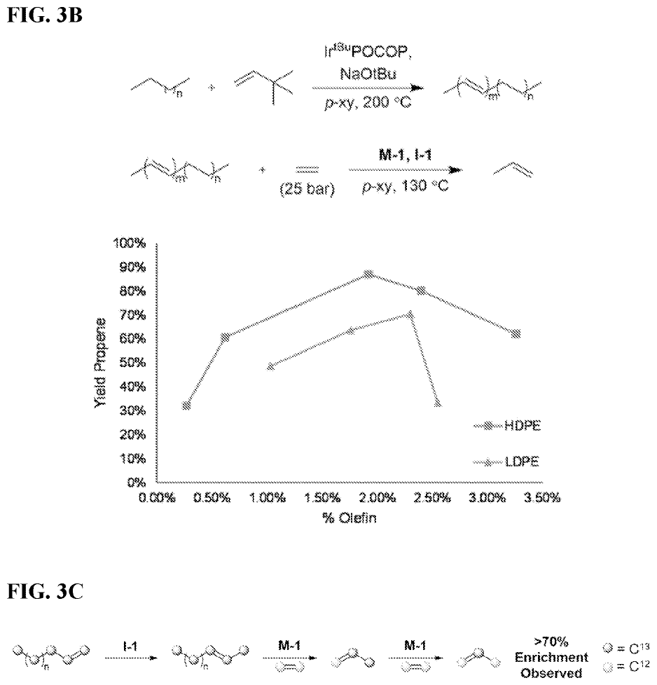

Having shown we could generate long-chain alkenes from PE, we investigated conditions for IE of the resulting chains. IE of hydrocarbons is known for the conversion of unsaturated alkylarenes to vinylarenes and for the conversion of 1-octadecene to propene at low conversions. We sought an IE that would convert the long-chain PE fragments to propene with high conversions by seeking conditions for high conversion of 1-octadecene (1a) (19). Several combinations of complexes known to catalyze either olefin metathesis or isomerization were tested, and the results are summarized in FIG. 3A. The reaction of octadecene with ethylene catalyzed by the combination of Hoveyda-Grubbs catalyst M-1 (6 mol % relative to alkene chains) and the dimeric Pd(I) bromide catalyst I-1 (3 mol %) at 60° C. for 16 h formed propene in 95% yield.

Catalyst systems created from other combinations of individual metathesis and isomerization catalysts led to lower yields of propene. The system containing M-3 and I-1 was the next most active, and combinations with other metathesis catalysts and I-1 led to lower yields. The reactions also occurred in lower yields with the combination of M-1 and other isomerization catalysts we tested. All the isomerization catalysts furnished exclusively internal octadecenes from 1-octadecene in high or quantitative yield after 16 h at 60° C. in p-xylene, implying that incompatibility of the isomerization catalysts with the metathesis catalyst or with ethylene is the cause of the lower yields of propene with isomerization catalysts 12-14 (see Table 4 for details).

TABLE 4
1-Octadecene isomerization.

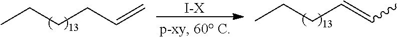

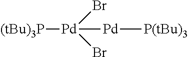

I-1

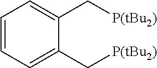

I-2
[Rh(cod)₂]BF₄
I-3

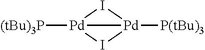

I-4

| Catalyst | % Internal Olefins (NMR) | % Terminal Olefin (NMR) |
|---|---|---|
| I-1 | >95% | — |
| I-2 | >95% | — |
| I-3 | >95% | — |
| I-4 | 79% | 11% |

The free energy of isomerizing ethenolysis is estimated to be negative, but small (−1.52 kcal/mol for each propene formed under standard conditions). Thus, our standard reactions that gave 95% yield of propene were conducted in a pressure vessel with sufficient head space to provide a large excess (64 equiv per methylene unit) of ethylene in the vessel to favor full consumption of the alkene chain. Consistent with the estimated thermodynamics, reactions with a smaller headspace providing just 3.5 equiv of ethylene in the system also led to a high, albeit slightly lower, 72±6% yield of propene.

Finally, we tested the formation of propene from ethylene and the unsaturated PEs we produced by dehydrogenation. The reactions of ethylene with the material from HDPE catalyzed by the two complexes in our IE system formed propene in yields shown in FIG. 3C. Reactions with HDPE containing only 0.68% unsaturation formed propene in nearly 60% yield. Reaction of the polymer in which 1.9% of the monomer units were unsaturated led to the formation of propene in 87% yield. The conversion of unsaturated LDPE to propene also occurred under these conditions in yields up to 70%. Thus, the DIE process we disclose converts polyethylene and ethylene to propene in high yield.

We considered that ethylene could undergo dimerization or oligomerization, followed by ethenolysis to form propene without contribution from the polymer chains. Indeed, we did observe this background reactivity in the absence of long-chain olefins to form 0.13 mmol of propene under our reaction conditions after 16 h at 60° C. and to form 1.01 mmol of propene after the same time at 130° C. Thus, to determine whether the observed propene from the reactions with PE originated from dehydrogenated polymer or from the background conversion of ethylene to propene, we designed an isotope labeling experiment whereby the production of propene could be observed more directly. Because both dehydrogenation and IE scramble ²H labels, we conducted experiments with ¹³C-labeled PE to clearly assess the extent of formation of propene from polymer. The propene arising from dehydrogenated PE by the proposed scheme would consist entirely of the singly ¹³C labeled isotopomer. This isotopic composition would be observed because scrambling would occur at the termini of propene by olefin metathesis during the IE but scrambling at the 2-position would not (FIG. 3C). Accordingly, we subjected ¹³C-labeled (99.9% ¹³C) HDPE to DIE and characterized the resulting propene by mass spectrometry. The mass spectrum revealed an isotopic enrichment of the propene greater than 70%, demonstrating that the majority of propene observed from the DIE of PE results from polymer chain degradation.

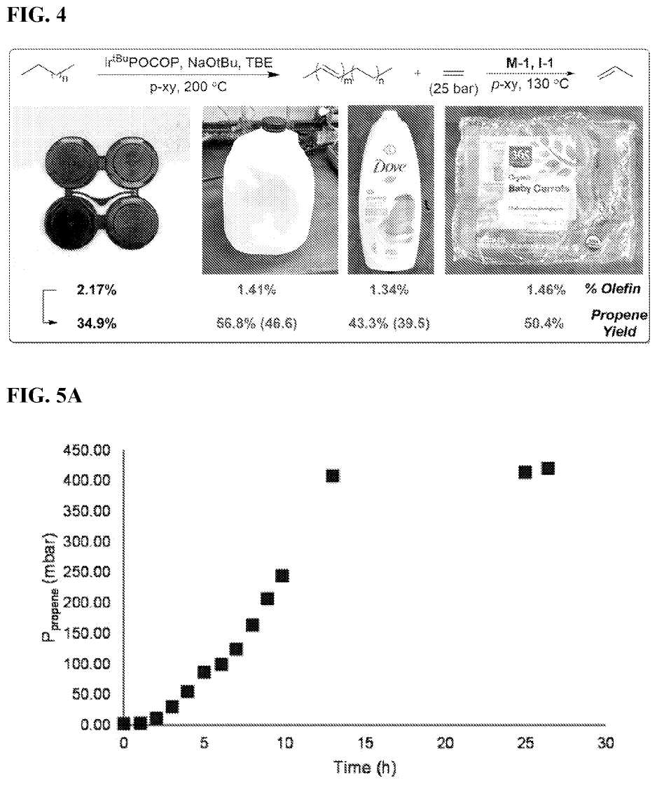

We also tested the potential to conduct DIE on samples of post-consumer PE. The results of the DIE of these post-consumer samples are displayed in FIG. 4. Substantial yields of propene were observed in all cases. Moreover, HDPE from a milk jug yielded 57% propene, which is only 20% lower than the yield from laboratory-grade HDPE with a similar degree of unsaturation, and LDPE food packaging generated over 50% yield of propene. These results show that a post-consumer product comprising HDPE or LDPE can be converted to propene by this type of chemical recycling.

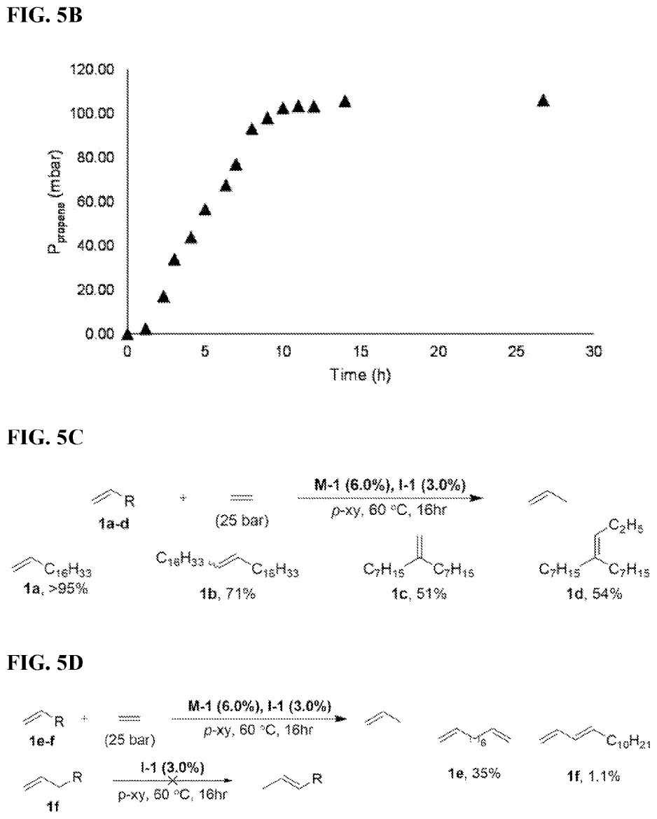

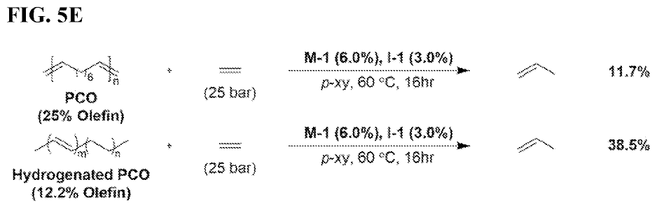

The time courses of reactions for degrading octadecene at 60° C. and degrading HDPE at 130° C. are displayed in FIG. 5A and FIG. 5B. These data revealed a lengthy induction period for the reaction of octadecene at 60° C., presumably due to the multiple stages of catalyst activation necessary for the reaction to occur at an appreciable rate. After the initiation period, the reaction proceeded at a constant rate until substrate was depleted. The reaction of the unsaturated HDPE at 130° C. occurred with a shorter induction period despite the need to first cleave the internal alkenes of the unsaturated HDPE, and this reaction occurred at a constant rate up to 85% of the total conversion. Presumably the higher temperature diminishes the induction period. The linear formation of propene suggests that the process occurs by rate-determining reaction with the excess ethylene.

Additional data we gathered on the degradation of PE by DIE point to future challenges. First, samples of LDPE consistently furnished propene in lower yields than HDPE of identical unsaturation. We hypothesized that the significant degree of branching in LDPE led to the formation of trisubstituted alkenes during IE, and these more hindered alkenes underwent slower isomerization or slower ethenolysis. To test this hypothesis, we synthesized several substituted olefins and subjected them to IE under conditions identical to those we used for the IE of 1-octadecene (FIG. 5C). The IE of substrates 1a-1d demonstrate a clear trend of decreasing propene yield with increasing olefin substitution.

Second, our experiments with laboratory-grade PE (see FIG. 3B) showed that a high degree of unsaturation of PE chains was detrimental to the yield of IE. We hypothesized from this observation that a high degree of polymer unsaturation leads to dienes and polyenes that isomerize during the IE process to conjugated dienes that could poison the catalyst. Because there are multiple sites of unsaturation on each polymer chain, I-1 could isomerize such olefins to conjugated 1,3-dienes within the polymer, and such conjugated dienes could poison one or both catalysts. To test this hypothesis, we conducted IE on one α,ω-diene 1d (1,9-decadiene) and one conjugated diene 1e (1,3-tetradecadiene) (FIG. 5D). These experiments showed that α-ω dienes separated by 6 methylene units (1e) formed propene in much higher yields than did 1,3-dienes. Consistent with our hypothesis, addition of a catalytic amounts of the I-1 to diene 1e without ethylene or the metathesis catalyst led to immediate formation of a black precipitate (FIG. 5D).

We assessed this hypothesis further by applying IE to poly(cyclooctene) (PCO). We synthesized PCO (Mₙ=5.3 kDa, Ð=1.64) by ring-opening metathesis polymerization of cyclooctene (27). IE of this substrate furnished propene in significantly diminished yield, but IE of a partially hydrogenated PCO (28) (with an olefin content of 12.2% versus 25% for the original polymer) furnished propene in 38.5% yield (FIG. 5E). We concluded from these data that the formation of 1,3-dienes in situ was responsible for the diminished yields of propene from the highly dehydrogenated polymers.

Overall, we have utilized DIE to deconstruct laboratory-grade samples of HDPE to form the single alkene propene in up to 80% yield and to deconstruct HDPE from post-consumer products in yields approaching 60%. In addition to its use as a monomer for commodity plastics, propene is used to form multiple commodity chemicals, including acrylic acid, acrylonitrile, and cumene (28). Although propene is produced primarily as a byproduct of steam cracking or fluid catalytic cracking, the increasing demand for propene since the 1990s has caused it to be prepared “on-purpose” through methanol-to-olefins technology, propane dehydrogenation, or the conproportionation of butene and ethylene (29). This “on purpose” production is only expected to increase as increasing populations and standards of living heighten the demand for polypropylene.

The constituent reactions of the DIE process we report on laboratory scale with homogeneous catalysts are closely related to reactions conducted on industrial scales with heterogeneous catalysts, and their combination draws analogies to the combination of reactions in the commodity-scale Shell Higher Olefin Process (SHOP) that forms long-chain alkenes from ethylene (29). The dehydrogenation of PE occurs with a homogeneous catalyst or with Pt/Zn supported on silica or Pt/Sn supported on alumina to yield dehydrogenated polyethylene with a level of unsaturation sufficient for the degradation of PE to propene. The olefin metathesis and olefin isomerization by which this degradation of the unsaturated polymer occurs are some of the most practiced industrial transformations, routinely performed on commodity scales with heterogeneous catalysts (30, 31). Thus, the eventual translation of such catalyst systems to DIE will allow the dehydrogenation and IE processes to be conducted in continuous flow with less ethylene and removing the propene formed. The separation of the propene product from the ethylene reactant also is conducted on industrial scale by various methods, including low-temperature and extractive distillation (32). Thus, this initial report charts a path by which a DIE sequence could render polyolefin waste a hydrocarbon feedstock to generate a commodity chemical for which the demand is comparable to the available supply of recovered polyolefins.

### REFERENCES FOR EXAMPLE 1

1. R. Geyer, J. R. Jambeck, K. L. Law, Science Advances 3, e1700782 (2017). 2. G. W. Coates, Y. D. Y. L. Getzler, Nat. Rev. Mat. 5, 501-516 (2020). 3. I. Vollmer et al., Angew. Chem. Int. Ed. 59, 15402-15423 (2020). 4. K. Ragaert, L. Delva, K. Van Geem, Waste Management 69, 24-58 (2017). 5. L. Chen et al., Chem 7, 137-145 (2021). 6. M. Zeng et al., ACS Sus. Chem. Eng. 9, 13926-13936 (2021). 7. T. J. Fazekas et al., Science 375, 545-550 (2022). 8. J. B. Williamson, W. L. Czaplyski, E. J. Alexanian, F. A. Leibfarth, Angew. Chem. Int. Ed. 57, 6261-6265 (2018). 9. L. T. J. Korley, T. H. Epps, B. A. Helms, A. J. Ryan, Science 373, 66-69 (2021). 10. M. Chu, Y. Liu, X. Lou, Q. Zhang, J. Chen, ACS Catalysis 12, 4659-4679 (2022). 11. D. P. Serrano, J. Aguado, J. M. Escola, Ind. Eng. Chem. Res. 39, 1177-1184 (2000). 12. K. L. Sánchez-Rivera, G. W. Huber, ACS Cent. Sci. 7, 17-19 (2021). 13. S. P. Ertem et al., Macromolecules 53, 6043-6055 (2020). 14. G. Celik et al., ACS Cent. Sci. 5, 1795-1803 (2019). 15. J. E. Rorrer, C. Troyano-Valls, G. T. Beckham, Y. Román-Leshkov, ACS Sus. Chem. Eng. 9, 11661-11666 (2021). 16. F. Zhang et al., Science 370, 437-441 (2020). 17. X. Jia, C. Qin, T. Friedberger, Z. Guan, Z. Huang, Science Advances 2, e1501591 (2016). 18. L. D. Ellis et al., ACS Sustainable Chemistry & Engineering 9, 623-628 (2021). 19. M. Nagyházi, Á. Lukács, G. Turczel, J. Hancsók, J. Valyon, A. Bényei, et al. Angew. Chem. Int. Ed. Article ASAP. DOI: 10.1002/anie.202204413 (Accessed Apr. 29, 2022). 20. S. Baader, D. M. Ohlmann, L. J. Gooßen, Chem. Eur. J. 19, 9807-9810 (2013). 21. A. Ray et al., Chem. Comm., 3388-3390 (2005). 22. A. Ray et al., J. Mol. Cat. A Chem. 256, 200-207 (2006). 23. Göttker-Schnetmann, P. White, M. Brookhart, J. Am. Chem. Soc. 126, 1804-1811 (2004). 24. S. Biswas et al., J. Am. Chem. Soc. 134, 13276-13295 (2012). 25. Y. Liu, K. Dong, M. Beller, S. Mecking, ACS Catalysis 8, 9232-9237 (2018). 26. S. He, K. R. Krishnamurthy, K. Seshan, in Catalysis: Volume 29. (The Royal Society of Chemistry, 2017), vol. 29, pp. 282-316. 27. R. Walker, R. M. Conrad, R. H. Grubbs, Macromolecules 42, 599-605 (2009). 28. S. Kobayashi, L. M. Pitet, M. A. Hillmyer, J. Am. Chem. Soc. 133, 5794-5797 (2011). 29. R. Schmidt et al., Hydrocarbons in Ullmann's Encyclopedia of Industrial Chemistry. pp. 1-74. 30. H. Zimmermann, Propene in Ullmann's Encyclopedia of Industrial Chemistry. pp. 00-00. 31. F. M. A. Geilen, G. Stochniol, S. Peitz, E. Schulte-Koerne, Butenes in Ullmann's Encyclopedia of Industrial Chemistry. pp. 1-13. 32. R. B. Eldridge, Industrial & Engineering Chemistry Research 32, 2208-2212 (1993). 33. Kalvet, K. J. Bonney, F. Schoenebeck, J. Org. Chem. 79, 12041-12046 (2014). 34. B. Inci, K. B. Wagener, J. Am. Chem. Soc. 133, 11872-11875 (2011). 35. Weast, R. C.; Company, C. R., Handbook of Chemistry and Physics: A Ready-reference Book of Chemical and Physical Data. (Chemical Rubber Company: Cleveland, ed. 53, 1972). 36. K. G. Joback, R. C. Reid, Chem. Eng. Commun. 57, 233-243 (1987).

### Example 2: Thermodynamics of IE

Thermochemical parameters for IE at standard conditions were calculated for α-olefins of varying chain lengths according to Hess' law (S4), substituting out Gibbs Free energy or entropy for enthalpy to determine the contributions of enthalpy and entropy and the effect of varying chain lengths on reaction thermodynamics. Free energies for small molecules were approximated by the Joback method, while standard enthalpies of formation and molar entropies were sourced from the NIST chemistry webbook (36), and thermodynamic parameters for polyethylene were sourced from Broadhurst (37).

$$Δ {H}_{f rxn}^{°} = \sum {n}_{p r o ducts} Δ {H}_{f products}^{°} - \sum {n}_{r e a c tants} Δ {H}_{f reactants}^{°} \qquad ( S4 )$$

in which n_{products} and n_{reactants} represent the stoichiometric coefficients of each product and reactant respectively, and ΔH_{f}°_{products} and ΔH_{f}°_{products} represent the enthalpy of formation for each product and reactant, and ΔH_{f}°_{r×n} represents this parameter for the global reaction.

### Example 3: Isomerizing Ethenolysis of 1-Octadecene with Heterogeneous Catalysts

### Catalyst Preparation

Re₂O₇/γ-Al₂O₃: This procedure was adapted from a procedure reported by Goldman and coworkers. See Goldman, A. S.; Roy, A. H.; Huang, Z.; Ahuja, R.; Schinski, W.; Brookhart, M. Science 312, 5771, 257-261 (2006). DOI: doi: 10.1126/science. 1123787 (stating: “In a vial, 1.20 g (4.47 mmol) of NH₄ReO₄ was dissolved in 30 ml distilled water. This solution was added to 10 g (98 mmol) of γ-Al₂O₃. The suspension was swirled by hand for about a minute, then allowed to stand undisturbed at room temperature for 30 min. This cycle of swirling and standing was repeated until all of the water was absorbed by the alumina. The solid was dried in a 120° C. oven overnight, then calcined at 550° C. for 3 hours under a flow of O₂ and cooled to room temperature under 02. The solid was brought into the drybox under vacuum to avoid exposure to moisture, and was stored under argon.”). This procedure produced Re₂O₇/γ-Al₂O₃ consisting of 8.4 wt % Re, or 0.45 mmol Re per gram of catalyst.

Na/γ-Al₂O₃: To a flame dried thick-walled Schlenk vessel were added sodium (160 mg, 6.96 mmol) and freshly calcined γ-Al₂O₃ (1.840 g). The vessel was then sealed and heated to 150° C. for 12 h, resulting in the formation of a gray-blue solid. The mixture was then cooled, stored under inert atmosphere, and used in isomerizing ethenolysis experiments without further modification.

### Isomerizing Ethenolysis

In a 25 mL quartz Parr reactor liner was combined 125 mg of Re₂O₇/γ-Al₂O₃ (0.056 mmol Re), SnBu₄ (10.0 mg, 0.029 mmol), and 1 mL toluene. The mixture was allowed to stir for 5 min, after which Na/γ-Al₂O₃ (60 mg) and 1-octadecene (126 mg, 0.500 mmol) were added to the stirring mixture. The liner was then placed in a 25 mL Parr reactor, which was sealed and charged with 10 and 25 bar of methane and ethylene respectively. The reactor was then allowed to stir at 125° C. for 24 h. The headspace was analyzed by GC-FID to quantify the yield of propene. The results of optimization experiments by which this procedure was conceived are summarized in the Table 5 below.

TABLE 5
Optimization of Heterogeneous IE.

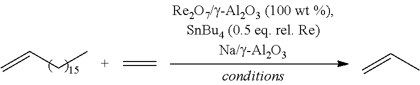

| Entry | P_{Ethylene} (bar) | T (° C.) | [Na] (wt %) | Solvent | Yield |
|---|---|---|---|---|---|
| 1 | 10 | 60 | 50 | p-xylene | 5.7 |
| 2 | 10 | 100 | 50 | p-xylene | 11.4 |
| 3 | 10 | 125 | 50 | p-xylene | 29.6 |
| 4 | 10 | 150 | 50 | p-xylene | 24.7 |
| 5 | 25 | 125 | 25 | p-xylene | 5.6 |
| 6 | 25 | 125 | 50 | p-xylene | 8.1 |
| 7 | 25 | 125 | 100 | p-xylene | 39.5 |
| 8 | 5 | 125 | 50 | p-xylene | 34.7 |
| 9 | 2 | 125 | 50 | p-xylene | 23.3 |
| 10^{a} | 25 | 125 | 50 | Mesitylene | 16.9 |
| 11^{a} | 25 | 125 | 50 | p-xylene | 23.3 |
| 12^{a} | 25 | 125 | 50 | Toluene | 86.5 |

^{a}These entries were conducted in a Parr reactor with an internal volume of 25 mL.

1. A method of degrading a substance, said method comprising:
(i) conducting one or more reaction(s) to form one or more olefin units in the substance;
(ii) subjecting, after step (i), to conditions for olefin metathesis; and
(iii) subjecting, after step (i), to conditions for olefin isomerization.

2. The method of claim 1, wherein step (i) comprises subjecting the substance to conditions for dehydrogenation.

3. A method of making a substituted or unsubstituted alkene, said method comprising subjecting a dehydrogenated polymer to conditions for metathesis and to conditions for olefin isomerization.

4. A method of making a substituted or unsubstituted alkene, said method comprising subjecting a first substance to conditions for olefin metathesis and to conditions for olefin isomerization; wherein:
(i) said first substance comprises at least 20 carbon atoms per molecule; or
(ii) prior to subjecting said first substance to conditions for olefin metathesis and to conditions for olefin isomerization, the method comprises subjecting a second substance to conditions for dehydrogenation, thereby forming said first substance.

5. The method of claim 4, wherein the substituted or unsubstituted alkene is a substituted or unsubstituted propene or substituted or unsubstituted dehydrogenated polyethylene.

6. (canceled)

7. A method of making an alkene, said method comprising mixing a dehydrogenated polymer with one or more olefin metathesis reagent(s) and one or more isomerization reagent(s); wherein:
(i) the dehydrogenated polymer comprises at least 20 carbon atoms; or
(ii) prior to said mixing, the method comprises mixing a polymer with one or more dehydrogenating reagent(s), thereby forming said dehydrogenated polymer.

8. A method of making a dehydrogenated polymer, said method comprising mixing a polymer with one or more dehydrogenating reagent(s), wherein
(i) a percentage of from 0.01% to 1.5% or from 1.7% to 2.3% or from 2.4% to 4.3% or 4.5% to 13.2% of monomer units in the dehydrogenated polymer are unsaturated;
(ii) the dehydrogenated polymer has a number average molecular weight of about 2,200 Da or greater;
(iii) the dehydrogenated polymer has a number average molecular weight of about 2,200 Da to about 6,500 Da and a percentage of from 0.01% to 10% of monomer units are unsaturated; or
(iv) the dehydrogenated polymer has a number average molecular weight of about 1,000 Da to about 500,000 Da and a percentage of from 0.01% to 1.5% or from 1.7% to 2.3% or from 2.4% to 4.3% or from 4.5% to 10% of monomer units in the dehydrogenated polymer are unsaturated.

9. The method of claim 8, wherein the dehydrogenated polymer has a number average molecular weight of from about 2,200 Da to about 6,500 Da or from about 6,700 Da to about 15,000 Da.

10. (canceled)

11. The method of claim 8, further comprising mixing the dehydrogenated polymer with one or more olefin metathesis reagent(s) and one or more isomerization reagent(s), thereby forming an alkene.

12. The method of claim 7, wherein the dehydrogenated polymer is a substituted or unsubstituted dehydrogenated polyethylene or a substituted or unsubstituted propene.

13. (canceled)

14. The method of claim 7, wherein the dehydrogenated polymer, the one or more olefin metathesis reagent(s), and the one or more isomerization reagent(s) are mixed simultaneously, prior to adding the one or more isomerization reagent(s) or prior to adding the one or more olefin metathesis reagent(s) and the one or more isomerization reagent(s).

15. (canceled)

16. (canceled)

17. The method of claim 7, wherein the one or more olefin metathesis reagent(s) comprises an ethenolysis catalyst.

18. The method of claim 7, wherein the one or more olefin metathesis reagent(s) comprises one or more homogeneous olefin metathesis catalyst(s) or one or more heterogeneous olefin metathesis catalyst(s).

19. The method of claim 18, wherein the one or more olefin metathesis reagent(s) further comprises ethylene.

20. The method of claim 18, wherein the homogeneous olefin metathesis catalyst is

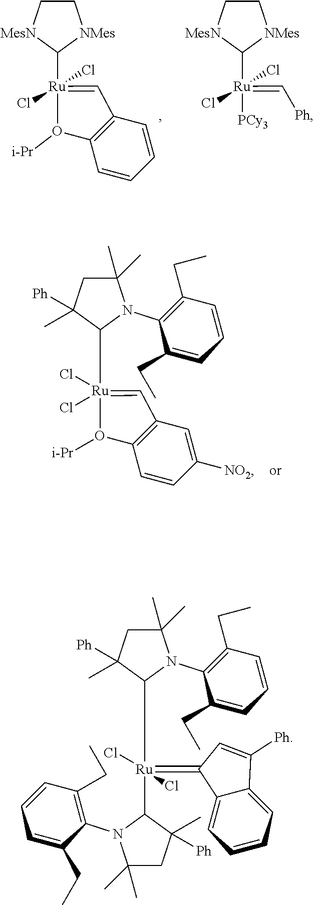

21. (canceled)

22. The method of claim 18, wherein the heterogeneous olefin metathesis catalyst is Re₂O₇/Al₂O₃ or WO₃/Al₂O₃.

23. The method of claim 7, wherein the one or more isomerization reagent(s) comprises one or more homogeneous isomerization catalyst(s) or one or more heterogeneous isomerization catalyst(s).

24. The method of claim 23, wherein the one or more homogeneous isomerization catalyst(s) is selected from: 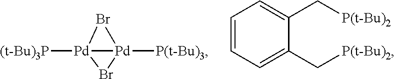
[Rh(cod)₂]BF₄ and

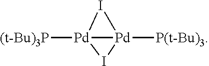

25. (canceled)

26-50. (canceled)

27. A substituted or unsubstituted dehydrogenated polyethylene, wherein a percentage of from 0.01% to 1.5% or from 1.7% to 2.3% or from 2.4% to 4.3% or from 4.5% to 13.2% of monomer units are unsaturated.

28. (canceled)

29. A substituted or unsubstituted dehydrogenated polyethylene, having a number average molecular weight of 2,200 Da or greater.

30-65. (canceled)
